(19) United States
(12) Patent Application Publication
(10) Pub. No.: US 2008/0063628 A1
(43) Pub. Date: Mar. 13, 2008

(54) METHODS TO PROMOTE CELL DIFFERENTIATION

(76) Inventors: Janet E. Davis, Branchburg, NJ (US); Ramie Fung, Flemington, NJ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/530,622

(22) Filed: Sep. 11, 2006

(51) Int. Cl. A61K 35/12; C12N 5/02

(52) U.S. Cl. 424/93.7; 435/377

(57) ABSTRACT

A method for promoting the differentiation of cells by contacting cells with a chromatin-remodeling agent to increase the expression of a transcriptional regulator.

### FIELD OF THE INVENTION

This invention relates to a method for inducing the differentiation of cells. In particular, this invention relates to methods that induce cells to differentiate into a pancreatic hormone-secreting cell or into a P-cell lineage. This invention also provides methods and compositions for utilizing such cells in the therapeutic treatment of diabetes.

### BACKGROUND

Loss of organ function can result from congenital defects, injury or disease. One example of a disease causing loss of organ function is diabetes mellitus, or diabetes. The majority of diabetes cases fall into two clinical types: Type 1, also known as juvenile-onset diabetes or insulin dependent diabetes mellitus (IDDM); and Type 2, also known as adult-onset diabetes. A common method of treatment of Type 1 diabetes involves the exogenous administration of insulin, typically by injection with either a syringe or a pump. This method does not completely normalize blood glucose levels and is often associated with an increased risk of hyperblycemia or hypoglycemia. More effective glycemic control can be achieved if the function of the pancreas can be restored or rejuvenated via transplantation or cell-based therapies.

There are many transplantation therapies currently used to treat diabetes. One such treatment involves transplanting isolated islets of Langerhans into a diabetic patient. One challenge to human islet transplantation has been the lack of sufficient numbers of pancreata and islets to treat the large number of diabetic patients.

Alternative sources of cellular material for transplantation may include, for example, cells derived from other tissues such as, for example, chorionic villus, amniotic fluid, and bone marrow. These other tissues may be fetal or embryonic tissues. In addition, the endocrine cells of the islets of Langerhans, including β-cells, are constantly turning over by processes of apoptosis and proliferation of new islet cells (neogenesis). As such, the pancreas is thought to be a source of undifferentiated cells that are capable of differentiating into pancreatic hormone producing cells.

However, one challenge of these cellular approaches has been the ability of these cells to differentiate into a β-cell lineage or a pancreatic hormone-secreting cell. Such differentiation involves changes in gene expression.

Mechanisms for cellular differentiation: Gene expression is the combined process of the transcription of a gene into mRNA, the processing of that mRNA, and its translation into protein (for protein-encoding genes). A comparison of the gene-expression patterns of cells from the pancreas, a site for secretion of digestive enzymes and hormones, and the liver, a site of lipid transport and energy transduction, reveals marked differences in the genes that are highly expressed, a difference consistent with the physiological roles of these tissues. For example, insulin gene expression in a mammal is restricted to the β-cells of the pancreas through control mechanisms mediated in part by specific transcription factors including MafA, and NeuroD. In other cells of the body, the pancreatic hormones, such as, for example, insulin, as well as other specific peptidase genes are trancriptionally silent.

DNA is never found as a naked molecule in animal or plant cell nuclei. DNA is always found in association with proteins and other molecules. The molecules include, for example, histone proteins (soluble in acid solutions), HMG proteins (soluble in neutral saline), residual proteins (soluble in concentrated urea solutions), phosphoproteins (soluble in basic solutions), RNA species (soluble in aqueous phenol solutions), and lipid species (soluble in chloroform-methanol solutions). Chromatin is that portion of the cell nucleus that contains the entire DNA localized in the nucleus of animal or plant cells.

When cells divide, the chromatin is seen as distinct chromosomes which duplicate with an equal partition of each set of chromosomes then traveling to each of the new daughter cells. When the chromosomes reach the new cells, they begin to unravel into long thin extended 10 nm microfibrils, called euchromatin, or condensed coiled masses, called heterochromatin. The study of euchromatin and heterochromatin has revealed that RNA synthesis occurs only in euchromatin and not in heterochromatin.

Covalent modification of histone proteins has been implicated in the regulation of gene expression. Reversible acetylation of histone proteins can combine with DNA methylation and other modifications to generate an epigenetic code of altered chromatin structure and function. The acetylation state of histones and other proteins is dynamically regulated by the competing actions of acetyltransferases and deacetylases. Hypoacetylated histones promote chromatin condensation and are associated with transcriptionally silent loci, wherein access of the DNA to transcription factors or the transcriptional apparatus is limited. Such alterations to chromatin may play a seminal role in tissue differentiation by determining the complement of genes expressed within individual cell lineages.

Factors that control pancreatic development: The homeodomain protein PDX-1 (Pancreatic and Duodenal Homeobox gene-1, also known as IDX-1, IPF-1, STF-1 or IUF-1) plays a central role in regulating pancreatic islet development and function. PDX-1 regulates transcription of the genes associated with β-cell identity, including insulin, glucokinase, islet amyloid polypeptide, and glucose transporter type 2 (GLUT2).

US20050090465 states the ectopic expression of PDX-1 in liver and skin induces a pancreatic islet cell phenotype in liver and skin cells and results in the expression, production, and processing of pancreatic hormones.

US20040002447 provides methods for inducing insulin gene expression in cells. In some embodiments, the methods comprise the steps of: (i) providing a cell that expresses a PDX-1 polynucleotide; and (ii) contacting the cell with a histone deacetylase inhibitor, thereby inducing insulin gene expression in the cells.

The methods disclosed in US20050090465 and US20040002447 require the ectopic expression of PDX-1 in order to induce insulin gene expression in cells. Thus, there remains a significant need to develop methods of generating pancreatic hormone-secreting cells from an abundant cell source that does not also require the ectopic expression of PDX-1.

### SUMMARY

The present invention includes methods that promote the differentiation of cells by altering the expression of genes within the cells. In one embodiment, the genes may be required for the differentiation of a desired cell lineage. Alternatively, the genes may be associated with the function of a desired cell lineage. In one embodiment, the expression of genes required for the differentiation and the function of a desired cell lineage may be altered.

The cells to be differentiated may themselves be fully differentiated cells of another cell lineage, or they may be partially differentiated progenitor cells, or they may be undifferentiated progenitor cells.

In one embodiment, the differentiation of cells may be promoted by contacting the cells with at least one chromatin-remodeling agent. Cells may be contacted with a single treatment of at least one chromatin-remodeling agent. In an alternate embodiment, the cells may be contacted with multiple treatments of the at least one chromatin-remodeling agent. The multiple treatments may be with the same agent, or a different agent.

The cells may not express the homeodomain protein PDX-1. Alternatively, the cells may have lost the expression of PDX-1 during culture in vitro. Contacting the cells with at least one chromatin-remodeling agent increases or restores the expression of PDX-1.

The present invention includes methods that cause a cell to differentiate into a pancreatic hormone-producing cell, or a cell of the β-cell lineage, by contacting the cell with at least one chromatin-remodeling agent.

In one embodiment, differentiation may be promoted by increasing the expression of at least one differentiation-related gene, selected from the group consisting of PDX-1, Sox-17, and HNF-3 beta. Alternatively, differentiation may be promoted by increasing the expression of at least one pancreatic hormone. Alternatively, differentiation may be promoted by increasing the expression of at least one differentiation-related gene and at least one pancreatic hormone.

In one embodiment, the at least one chromatin-remodeling agent induces changes in the expression of at least one differentiation-related gene and at least one pancreatic hormone. Alternatively, changes in the expression of at least one pancreatic hormone are mediated by contacting the cells with at least one other factor that promotes the differentiation of cells.

In one embodiment the at least one chromatin-remodeling agent may be an inhibitor of histone deacetylase activity. The inhibitor of histone deacetylase activity may be selected from the group consisting of butyrates, hydroxamic acids, cyclic peptides and benzamides. In some embodiments, the inhibitor of histone deacetylase activity may be selected from the group consisting of 4-phenylbutyrate, sodium butyrate, trichostatin A, suberoyl anilide hydroxamic acid (SAHA), oxamflatin, trapoxin B, FR901228, apicidin, chlamydocin, depuecin, scriptaid, depsipeptide, and N-acetyldinaline.

### BRIEF DESCRIPTION OF THE DRAWINGS

FIG. 1 outlines the postulated covalent modifications of histones.

FIG. 2 shows the effects of histone deacetylase inhibitor treatment on gene expression in Panc-1 Cells and neonatal fibroblasts. The data shown reflect the effect of 2.5 μM or 5 μM trichostatin A treatment on the expression of glucagon (panel a), Sox-17 (panel b), Pdx-1 (panel c) and HNF-3 beta (panel d). Untreated cells are shown as a negative control for comparison. The experimental procedure is outlined in Example 1.

FIG. 3 shows changes in gene expression in amniotic fluid-derived cells over time, following addition of 1.25 μM trichostatin A. The data shown reflect the relative expression of insulin (panel a), Sox-17 (panel b), Pdx-1 (panel c) and HNF-3 beta (panel d) compared to an untreated control. The experimental procedure is outlined in Example 2.

FIG. 4 shows changes in gene expression in late passage pancreatic-derived stromal cells over time following addition of 2.5 μM trichostatin A. The data shown reflect the relative expression of Sox-17 (panel a), HNF-3 beta and Pdx-1 (panel b), and glucagon (panel c) compared to an untreated control. The experimental procedure is outlined in Example 3.

FIG. 5 shows the changes in gene expression in amniotic fluid-derived cells with time following continuous chronic treatment with trichostatin A. The data shown reflect the relative expression of glucagon (panel a), HNF-3 beta (panel b), insulin (panel c), Pdx-1 (panel d) and Sox-17 (panel e) compared to an untreated control. The experimental procedure is outlined in Example 4.

FIG. 6 shows the changes in gene expression in late passage pancreatic-derived stromal cells with time following continuous chronic treatment with trichostatin A. The data shown reflect the relative expression of glucagon (panel a), HNF-3 beta (panel b), Pdx-1 (panel c) and Sox-17 (panel d) compared to an untreated control. The experimental procedure is outlined in Example 5.

### DETAILED DESCRIPTION OF THE INVENTION

For clarity of disclosure, and not by way of limitation, the detailed description of the invention is divided into the following subsections that describe or illustrate certain features, embodiments, or applications of the present invention.

### Cells Useful in the Present Invention

Cells suitable for use in the methods of the present invention may be obtained from tissues such as, for example, bone marrow, umbilical cord blood, amniotic membrane, amniotic fluid, placenta, skin, fat, muscle, vasculature, liver, pancreas, or peripheral blood using methods that are well known in the art. The cells may be fully differentiated, or they may be partially differentiated progenitor cells, or they may be undifferentiated progenitor cells. It is also possible to use cells, either fully or partially differentiated or undifferentiated, derived from umbilical cord tissue and/or embryonic tissue.

Differentiation is the process by which an unspecialized (“uncommitted”) or less specialized cell acquires the features of a specialized cell, such as, for example, a nerve cell or a muscle cell. A differentiated cell is one that has taken on a more specialized (“committed”) position within the lineage of a cell. The term committed, when applied to the process of differentiation, refers to a cell that has proceeded in the differentiation pathway to a point where, under normal circumstances, it will continue to differentiate into a specific cell type or subset of cell types and cannot, under normal circumstances, differentiate into a different cell type or revert to a less differentiated cell type. De-differentiation refers to the process by which a cell reverts to a less specialized (or committed) position within the lineage of a cell. As used herein, the lineage of a cell defines the heredity of the cell, i.e. which cells it came from and what cells it can give rise to. The lineage of a cell places the cell within a hereditary scheme of development and differentiation.

A progenitor cell is a cell that has the capacity to create progeny that are more differentiated than itself and yet retains the capacity to replenish the pool of progenitors. By that definition, stem cells themselves are also progenitor cells, as are the more immediate precursors to terminally differentiated cells.

Isolation of a population of cells may be achieved using monoclonal antibodies specific to proteins expressed on the surface of the cells. The monoclonal antibodies may be adhered to a substrate to facilitate the separation of the bound cells. The methods that may be used to isolate cells suitable for use in the present invention may be chosen by one of ordinary skill in the art. Examples of such methods are taught in U.S. Pat. No. 6,087,113, U.S. Pat. No. 6,261,549, U.S. Pat. No. 5,914,262, U.S. Pat. No. 5,908,782, and US20040058412.

Cells may be characterized, for example, by growth characteristics (e.g., population doubling capability, doubling time, passages to senescence), karyotype analysis (e.g., normal karyotype; maternal or neonatal lineage), flow cytometry (e.g., FACS analysis), immunohistochemistry and/or immunocytochemistry (e.g., for detection of epitopes), gene expression profiling (e.g., gene chip arrays; polymerase chain reaction (for example, reverse transcriptase PCR, real time PCR, and conventional PCR)), protein arrays, protein secretion (e.g., by plasma clotting assay or analysis of PDC-conditioned medium, for example, by Enzyme Linked Immuno-Sorbent Assay (ELISA)), mixed lymphocyte reaction (e.g., as measured by the stimulation of PBMCs), and/or other methods known in the art.

Cells suitable for use in the methods of the present invention may also include cells obtained from commercial sources, such as, for example human mesenchymal stem cells sold under the trade name POIETICS™ (Cat. No PT-2501, Cambrex). These mesenchymal stem cells are positive for the expression of the following markers: CD29, CD44, CD105 and CD166. The cells are negative for the expression of the markers CD14, CD34 and CD45.

In one aspect of the present invention, the cells may be pancreatic-derived stromal cells. These cells may be isolated by a multi-stage method, which is described in Example 13. Alternatively, the pancreatic-derived stromal cells may be isolated by any suitable method known to those of skill in the art. Examples of suitable isolation methods are taught in US2003/0082155, U.S. Pat. No. 5,834,308, U.S. Pat. No. 6,001,647, U.S. Pat. No. 6,703,017, U.S. Pat. No. 6,815,203, WO2004/011621.

In one aspect of the present invention, the cells may be amniotic fluid-derived cells. These cells may be isolated by a multi-stage method that is described in detail in Example 14. Alternatively, the amniotic fluid-derived cells may be isolated by any suitable method known to those of skill in the art. Examples of suitable isolation methods are taught in WO2003/042405, US2005/0054093, in't Anker et al, Blood 102, 1548-1549, 2003, Tsai et al, Human Reproduction 19, 1450-1456, 2004.

Isolated cells or tissue from which cells are obtained may be used to initiate, or seed, cell cultures. Isolated cells may be transferred to sterile tissue culture vessels, either uncoated or coated with extracellular matrix or ligands such as laminin, collagen (native, denatured or crosslinked), gelatin, fibronectin, and other extracellular matrix proteins. Cells may be cultured in any culture medium capable of sustaining growth of the cells, such as, for example, DMEM (high or low glucose), advanced DMEM, DMEM/MCDB 201, Eagle's basal medium, Ham's F10 medium (F10), Ham's F-12 medium (F12), Iscove's modified Dulbecco's-17 medium, Mesenchymal Stem Cell Growth Medium (MSCGM), DMEM/F12, RPMI 1640, and CELL-GRO-FREE. The culture medium may be supplemented with one or more components, including, for example, fetal bovine serum (FBS); equine serum (ES); human serum (HS); beta-mercaptoethanol (BME or 2-ME); one or more growth factors (for example, platelet-derived growth factor (PDGF), epidermal growth factor (EGF), fibroblast growth factor (FGF), vascular endothelial growth factor (VEGF), insulin-like growth factor-1 (IGF-1), leukocyte inhibitory factor (LIF) and erythropoietin (EPO)); amino acids, including L-glutamine and L-valine; and one or more antibiotic and/or antimycotic agents to control microbial contamination (such as, for example, penicillin G. streptomycin sulfate, amphotericin B. gentamicin, and nystatin, either alone or in combination). The cells may be seeded in culture vessels at a density to allow cell growth.

Methods for the selection of the most appropriate culture medium, medium preparation, and cell culture techniques are well known in the art and are described in a variety of sources, including Doyle et al., (eds.), 1995, CELL &TISSUE CULTURE: LABORATORY PROCEDURES, John Wiley & Sons, Chichester; and Ho and Wang (eds.), 1991, ANIMAL CELL BIOREACTORS, Butterworth-Heinemann, Boston.

Cells suitable for use in the present invention may be expanded by culturing in a defined growth media containing at least one factor that stimulates the proliferation of the cells. The at least one factor may include, for example, nicotinamide, members of the TGF-β family, including TGF-β 1, 2, and 3, bone morphogenic proteins (BMP-2, -4, 6, -7, -11, -12, and -13), serum albumin, members of the fibroblast growth factor family, platelet-derived growth factor-AA, and -BB, platelet rich plasma, insulin growth factor (IGF-I, -II) growth differentiation factor (GDF-5, -6, -8, -10, 11), glucagon like peptide-I and -II (GLP-I and -II), GLP-I and GLP-II mimetobody, Exendin-4, retinoic acid, parathyroid hormone, insulin, progesterone, aprotinin, hydrocortisone, ethanolamine, beta mercaptoethanol, epidermal growth factor (EGF), gastrin I and II, copper chelators such as triethylene pentamine, TGF-β, forskolin, sodium butyrate, activin, betacellulin, noggin, neuron growth factor, nodal, insulin/transferrin/selenium (ITS), hepatocyte growth factor (HGF), keratinocyte growth factor (KGF), bovine pituitary extract, islet neogenesis-associated protein (INGAP), proteasome inhibitors, notch pathway inhibitors, sonic hedgehog inhibitors, or combinations thereof. Alternatively, cells suitable for use in the present invention may be expanded by culturing in conditioned media. By “conditioned media” is meant that a population of cells is grown in a basic defined cell culture medium and contributes soluble factors to the medium. In one such use, the cells are removed from the medium while the soluble factors the cells produce remain. This medium is then used to nourish a different population of cells.

### Characterization of the Cells of the Present Invention

Methods for assessing expression of genes, via protein or nucleic acid levels in cultured or isolated cells are standard in the art. These include real-time polymerase chain reaction (RT-PCR), see, for example, the methods described in Example 15, Northern blots, in situ hybridization (see, for example, Current Protocols in Molecular Biology (Ausubel et al., eds. 2001 supplement)), Western blotting, and for markers that are accessible in intact cells, flow cytometry analysis (FACS) (see, e.g., Harlow and Lane, Using Antibodies: A Laboratory Manual, New York: Cold Spring Harbor Laboratory Press (1998), and the methods described in Example 16) and immunoassays, such as immunohistochemical analysis of sectioned material (See, for example, the methods described in Example 17).

Examples of antibodies useful for detecting certain protein markers are listed in Table XI A&B. It should be noted that other antibodies directed to the same markers that are recognized by the antibodies listed in Table XI A&B are available, or can be readily developed. Such other antibodies can also be employed for assessing expression of markers in the cells isolated in accordance with the present invention.

Characteristics of cells of the β-cell lineage are well known to those skilled in the art, and additional characteristics of the β-cell lineage continue to be identified. These characteristics can be used to confirm that the cells have differentiated to acquire the properties characteristic of the β-cell lineage. β-cell lineage specific characteristics include the expression of one or more transcription factors such as, for example, PDX-1, NGN-3, Hlxb9, Nkx6, Isl-1, Pax6, NeuroD, HNF-1a, HNF-6, HNF-3 beta, and MafA, among others. These transcription factors are well established in the art for identification of endocrine cells. See, for example, Edlund (Nature Reviews Genetics 3: 524-632 (2002)).

“Pancreatic hormone-secreting cell” refers to cells that express, or secrete at least one hormone selected from the list glucagon, somatostatin, or insulin.

### Differentiation of Cells by Chromatin-Remodeling

Differentiation of the cells useful in the present invention may be achieved by altering the expression of genes within the cells. This may be achieved by treating the cells with at least one agent that remodels the chromatin structure within the cells, such that a region of DNA containing active or potentially active genes is more loosely packaged, less condensed, and can be accessed for transcription.

Cells treated with a chromatin-remodeling agent may exhibit global changes in gene expression not restricted to any single gene or family of genes. The outcome may down-regulate some genes, up-regulate others, and may leave still other genes unchanged depending on the cell type, its differentiation stage, and responses over time to both the treatment protocol and environmental or other stimulatory signals.

Further complexity may arise where chromatin-remodeling agents alter expression of genes that themselves regulate other downstream genes, for example transcription factor genes. Finally, chromatin-remodeling agents may not affect all gene regulatory domains in an equivalent manner and therefore may not yield full gene expression commiserate with a fully differentiated cell. For example, a chromatin-remodeling agent may not alter gene enhancer regions, which operate bi-directionally at variable distances from promoter regions, to the same degree, and as a consequence, a gene may be turned on without achieving full expression.

The genes whose expression levels are altered by treatment with chromatin-remodeling agents may be required for the differentiation of a desired cell type, herein referred to as “differentiation-related” genes. Alternatively, the genes may be associated with the function of a desired cell type. The function may include, for example, secretion of insulin, in the case of a β-cell. The chromatin-remodeling agents may affect the expression of differentiation-related genes and genes associated with the function of a desired cell type simultaneously.

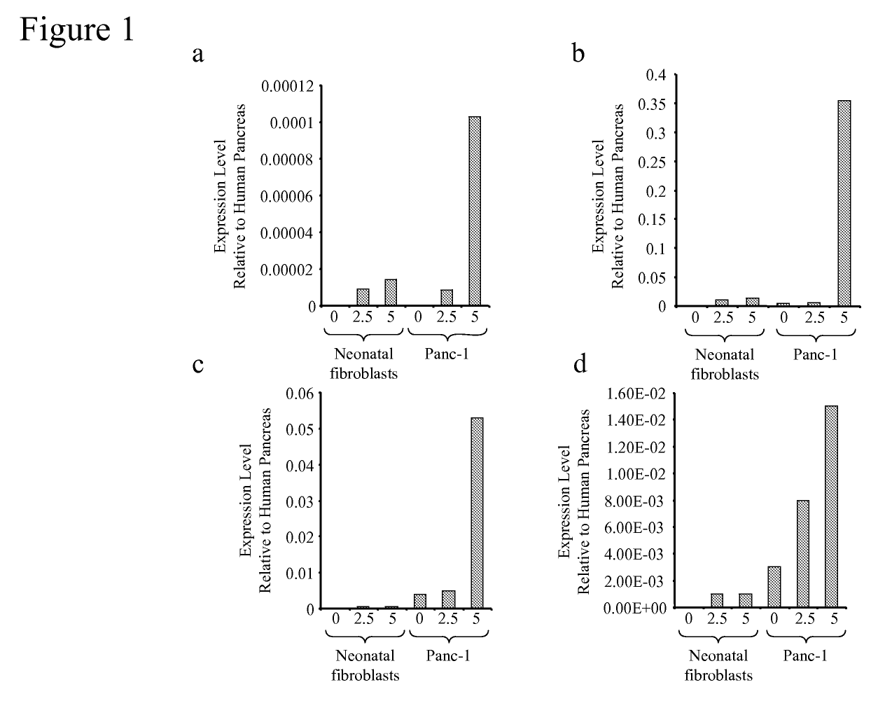

Chromatin remodeling may be achieved by direct covalent modification of histones. The covalent modification may be by acetylation, methylation, phosphorylation, ubiquitinylation and sumolylation. The possible covalent modifications to Histones are summarized in FIG. 1. The covalent modification may be achieved by adding at least one chromatin-remodeling agent that stimulates one, or all of these modifications. Alternatively, the at least one chromatin-remodeling agent may inhibit one, or more of these modifications.

The method of the present invention essentially involves:
Isolating a population of cells that does not express PDX-1, or has lost the expression of PDX-1 during culture in vitro,
Contacting the population of cells with at least one chromatin-remodeling agent,
Determining the subsequent changes in gene expression of the population of cells,
Culturing the treated population of cells in vitro.

The cells may require one, or more than one treatment of the at least one chromatin-remodeling agent. The more than one treatment may be with the same chromatin-remodeling agent, or a different chromatin-remodeling agent.

The concentration of the at least one chromatin-remodeling agent may vary, depending on the cell used, the choice of chromatin-remodeling agent or agents, the gene or genes whose expression levels are to be altered, the culture conditions, and the like. The at least one chromatin-remodeling agent may be contacted with the cells for up to about 48 hours, or up to about 24 hours, or up to about 12 hours, or up to about 6 hours, or up to about 4 hours, or up to about 2 hours, or up to about 1 hour.

Cells treated with at least one chromatin-remodeling agent may be treated with at least one other factor to promote the differentiation of the cells into a specific cell type. Factors may include, for example, nicotinamide, members of the TGF-β family, including TGF-β1, 2, and 3, bone morphogenic proteins (BMP-2, -4, 6, -7, -11, -12, and -13), serum albumin, fibroblast growth factor family, platelet-derived growth factor-AA, and -BB, platelet rich plasma, insulin growth factor (IGF-I, -II) growth differentiation factor (GDF-5, -6, -8, -10, 11), glucagon like peptide-I and -II (GLP-I and -II), GLP-I and GLP-II mimetobody, Exendin-4, retinoic acid, parathyroid hormone, insulin, progesterone, aprotinin, hydrocortisone, ethanolamine, beta mercaptoethanol, epidermal growth factor (EGF), gastrin I and II, copper chelators such as triethylene pentamine, TGF-β, forskolin, Na-Butyrate, activin, betacellulin, ITS, noggin, neurite growth factor, nodal, hepatocyte growth factor (HGF), keratinocyte growth factor (KGF), bovine pituitary extract, islet neogenesis-associated protein (INGAP), proteasome inhibitors, notch pathway inhibitors, sonic hedgehog inhibitors, or combinations thereof.

The combination and concentrations of growth factors, the length of culture, and other culture conditions can be optimized by those skilled in the art to achieve effective differentiation by, e.g., monitoring the percentage of cells that have differentiated into cells characteristic of the desired lineage. The one or more growth factors may be added in an amount sufficient to induce the differentiation of the cells of the present invention into cells bearing markers of a β-cell lineage over a time period of about one to four weeks.

### Chromatin-Remodeling Agents

In one aspect of the present invention, the chromatin-remodeling agent is a modulator of histone deacetylase activity. “Histone deacetylase” refers to enzymes that remove acetyl groups from histones. The modulator of histone deacetylase activity may enhance the activity of histone deacetylase enzymes, or it may inhibit the activity of histone deacetylase enzymes.

In one aspect, the inhibitor of histone deacetylase activity may be a delta dicarbonyl compound, such as, for example, compounds disclosed in European Patent Application EP1216986, having the general formula:

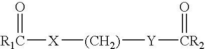

Wherein X is selected from the group consisting of oxygen, sulfur and N(R); wherein Y is selected from the group consisting of sulfur, N(R), and CH₂; wherein R is either H or CH₃; wherein R₁ and R₂ are the same or different and have the general formula:

—(CH₂)ₒ—(R₃)ₚ—(CH₂)_{q}—(R₄)ᵣ—(CH₂)ₛ—Z

Wherein R₃and R₄are the same or different and are selected from the group (CH═CH), (C≡C), sulfur and oxygen; wherein Z is selected from the group consisting of hydrogen and substituted or unsubstituted aryl, heteroaryl, cycloalkyl having the general formula CₙH₂ₙ-1 and alkoxy; wherein n is 3 or greater; and wherein o, p, q, r and s are the same or different and are each between 0 and 10.

In one aspect, the inhibitor of histone deacetylase activity may be a hydroxamate compound, such as, for example, compounds disclosed in WO0222577, having the general formula:

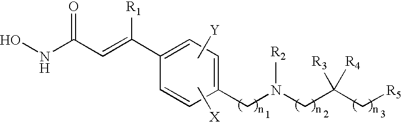

Wherein R, is H, halo, or a straight chain C₁-C₆ alkyl; R₂ is selected from H, C₁-C₁₀ alkyl, C₄-C₉ cycloalkyl, C₄-C₉ heterocycloalkyl, C₄-C₉ heterocycloalkylalkyl, cycloalkylalkyl, aryl, heteroaryl, arylalkyl, heteroarylalkyl, —(CH₂)ₙC(O)R₆, —(CH₂)ₙOC(O)R₆, amino acyl, HON—C(O)—CH═C(R₁)-aryl-alkyl- and —(CH₂)ₙR₇; R₃ and R₄ are the same or different and independently H, C₁-C₆ alkyl, acyl or acylamino, or R₃ and R₄ together with the carbon to which they are bound represent C=Q C═S, or C═NR₈, or R₂ together with the nitrogen to which it is bound and R₃ together with the carbon to which it is bound can form a C₄-C₉ heterocycloalkyl, a heteroaryl, a polyheteroaryl, a non-aromatic polyheterocycle, or a mixed aryl and non-aryl polyheterocycle ring; R₅ is selected from H, C₁-C₆ alkyl, C₄-C₉ cycloalkyl, C₄-C₉ heterocycloalkyl, acyl, aryl, heteroaryl, arylalkyl, heteroarylalkyl, aromatic polycycle, non-aromatic polycycle, mixed aryl and non-aryl polycycle, polyheteroaryl, non-aromatic polyheterocycle, and mixed aryl and non-aryl polyheterocycle; n, n₁, n₂ and n₃ are the same or different and independently selected from 0-6, when n₁ is 1-6, each carbon atom can be optionally and independently substituted with R₃ and/or R₄; X and Y are the same or different and independently selected from H, halo, C₁-C₄ alkyl, NO₂, C(O)R₁, OR₉, SR₉, CN, and N1R₁₀R₁₁; R₆ is selected from H, C₁-C₆ alkyl, C₄-C₉ cycloalkyl, C₄-C₉ heterocycloalkyl, cycloalkylalkyl, aryl, heteroaryl, arylalkyl, heteroarylalkyl, OR₁₂, and NR₁₃R₁₄; R₇ is selected from OR₁₅, SR₁₅, S(O)R₁₆, SO₂R₁₇, NR₁₃R₁₄, and NR₁₂SO₂R₆; R₈ is selected from H, OR₁₅, NR₁₃R₁₄, C₁-C₆ alkyl, C₄-C₉ cycloalkyl, C₄-C₉ heterocycloalkyl, aryl, heteroaryl, arylalkyl, and heteroarylalkyl; R₉ is selected from C₁-C₄ alkyl and C(O)-alkyl; R₁₀ and R₁₁ are the same or different and independently selected from H, C₁-C₄ alkyl, and —C(O)-alkyl; R₁₂ is selected from H, C₁-C₆ alkyl, C₄-C₉ cycloalkyl, C₄-C₉ heterocycloalkyl, C₄-C₉ heterocycloalkylalkyl, aryl, mixed aryl and non-aryl polycycle, heteroaryl, arylalkyl, and heteroarylalkyl; R₁₃ and R₁₄ are the same or different and independently selected from H, C₁-C₆, alkyl, C₄-C₉ cycloalkyl, C₄-C₉ heterocycloalkyl, aryl, heteroaryl, arylalkyl, heteroarylalkyl, amino acyl, or R₁₃ and R₁₄ together with the nitrogen to which they are bound are C₄-C₉ heterocycloalkyl, heteroaryl, polyheteroaryl, non-aromatic polyheterocycle or mixed aryl and non-aryl polyheterocycle; R₁₅ is selected from H, C₁-C₆ alkyl, C₄-C₉ cycloalkyl, C₄-C₉ heterocycloalkyl, aryl, heteroaryl, arylalkyl, heteroarylalkyl and (CH₂)ₘZ1R₁₂; R₁₆ is selected from C₁-C₆ alkyl, C₄-C₉ cycloalkyl, C₄-C₉ heterocycloalkyl, aryl, heteroaryl; polyheteroaryl, arylalkyl, heteroarylalkyl and (CH₂)ₘZ1R₁₂; R₁₇ is selected from C₁-C₆ alkyl, C₄-C₉ cycloalkyl, C₄-C₉ heterocycloalkyl, aryl, aromatic polycycle, heteroaryl, arylalkyl, heteroarylalkyl, polyheteroaryl and NR₁₃R₁₄; m is an integer selected from 0 to 6; and Z is selected from O, NR₁₃, S and S(O); or a pharmaceutically acceptable salt thereof.

In one aspect, the inhibitor of histone deacetylase activity may be a cyclic tetrapeptide compound, such as, for example, compounds disclosed in WO0021979, having the general formula:

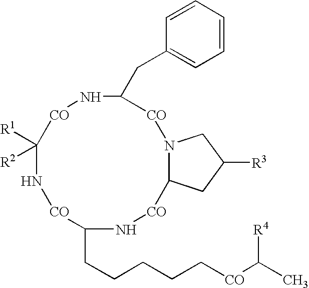

Wherein R1 is methyl, W is methyl or ethyl, W is hydrogen or methyl and W is hydroxy optionally having a hydroxy-protective group, providing that when W is hydrogen, W is ethyl.

In one aspect, the inhibitor of histone deacetylase activity may be a depsipeptide compound, such as, for example, compounds disclosed in WO0142282, having the general formula:

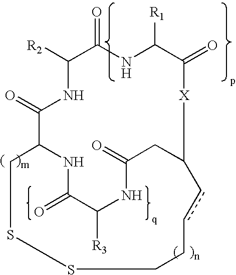

Wherein m is 1, 2, 3 or 4; n is 0, 1, 2 or 3; p and q are independently 1 or 2; X is O, NH or NR; R₁, R₂, and R₃ are the same or different and independently an amino acid side-chain moiety or an amino acid side-chain derivative; and R is a lower chain alkyl, aryl or arylalkyl moiety, with the proviso that the compound is not FR901228.

In one aspect, the inhibitor of histone deacetylase activity may be 6-(1,3-Dioxo-1H, 3H-benzo[de]isoquinolin-2-yl)-hexanoic acid hydroxyamide, termed “scriptaid”, as disclosed in WO0149290.

In one aspect, the inhibitor of histone deacetylase activity may be compounds having the general formula:

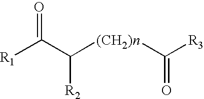

Wherein R₁, and R₂ are the same or different and are each a hydrophobic moiety; wherein R₃ is a hydroxamic acid, hydroxylamino, hydroxyl, amino, alkylamino, or alkyloxy group; and n is an integer from 3 to 10, or a pharmaceutically acceptable salt thereof, such as, for example, compounds disclosed in WO0118171.

In one aspect, the inhibitor of histone deacetylase activity may be a tricyclic alkylhydroxamate compound, such as, for example, compounds disclosed in WO2002085883, having the general formula:

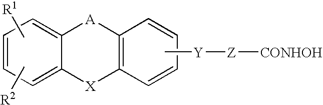

Wherein A denotes a bond, the groups —CH₂—O—, —CH₂—S—, —CH₂—CH₂—, or —NH—CO—; X denotes the group —NR³—, ═CO, or —CH(OH)—; Y denotes an oxygen atom, a sulfur atom, or the group —NR⁴—; Z denotes a straight chain alkylene group comprising 4, 5, 6, 7, or 8 carbon atoms, wherein one CH₂ group may be replaced by an oxygen or a sulfur atom, or wherein 2 carbon atoms form a C═C double bond, and which is either unsubstituted or substituted by one or two substituents selected from (₁₋₄C)alkyl and halogen atoms; R¹ and R² denote substituents independently selected from a hydrogen atom, halogen atoms, (₁₋₄C)alkyl, trifluoromethyl, hydroxy, (₁₋₄C)alkoxy, benzyloxy, (₁-₃C)alkylenedioxy, nitro, amino, (₁₋₄C)alkylamino, di[(₁₋₄C)alkyl]-amino, or (₁₋₄C)alkanoylamino groups; R³ and R⁴ independently denote hydrogen atoms or (₁₋₄C)alkyl groups; their enantiomers, diastereoisomers, racemates and mixtures thereof.

In one aspect, the inhibitor of histone deacetylase activity may be a tricyclic lactam or sultam derivative, such as, for example, compounds disclosed in WO2002062773, having the general formula:

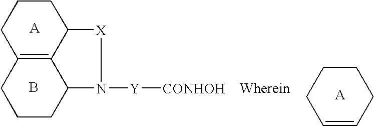

denotes a cyclohexenyl group or a phenyl group,

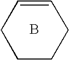

denotes a cyclohexenyl or a phenyl group which may be unsubstituted or substituted by one or more substituents independently selected from a halogen atom, a nitro group, an amino group, an (₁₋₄C)alkylamino group, a di[(₁₋₄C)alkyl]-amino group, or an (₁₋₄C)alkanoylamino group, X is a carbonyl group or a sulfonyl group, Y is a straight chain alkylene group comprising 5, 6, or 7 carbon atoms, wherein one CH₂ group may be replaced by an oxygen or a sulfur atom, or wherein 2 carbon atoms form a C═C double bond, and which is either unsubstituted or substituted by one or two substituents selected from (₁₋₄C)alkyl and halogen atoms, their enantiomers, diastereoisomers, racemates and mixtures thereof and pharmaceutically acceptable salts.

In one aspect, the inhibitor of histone deacetylase activity may be tetrahydropyridine derivative, such as, for example, compounds disclosed in WO2002051842, having the general formula:

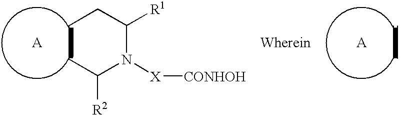

Denotes (a) a phenyl group which may be unsubstituted or substituted with 1, 2 or 3 substituents independently selected from a halogen atom, an (1-4C)alkyl-, trifluoromethyl-, hydroxy-, (1-4C)alkoxy-, benzyloxy-, (1-3C)alkylenedioxy-, nitro-, amino-, (1-4C)alkylamino-, di[(1-4C)alkyl]-amino-, (1-4C)alkanoyl-amino-, or a phenyl group, which may be unsubstituted or substituted by 1, 2, or 3 substituents independently selected from a chlorine atom, an (1-4C)alkyl-, trifluoromethyl-, hydroxy-, (1-4C)alkoxy-, (1-3C)alkylenedioxy-, nitro-, amino-, (1-4C)alkylamino-, di[(1-4C)alkyl]amino-, and a (1-4C)alkanoylamino group, or (b) denotes an indolyl group which may be unsubstituted or substituted with 1, 2 or 3 substituents independently selected from a halogen atom, an (1-4C)alkyl-, trifluoromethyl-, hydroxy-, (1-4C)alkoxy-, benzyloxy-, (1-3C)alkylenedioxy-, nitro-, amino-, (1-4C)alkylamino-, di[(1-4C)alkyl]amino-, or a (1-4C)alkanoylamino-group, R¹ and R² are the same as or different from each other and are a hydrogen atom, an (1-4C)alkyl-, a trifluoromethyl group, or an aryl group, X is a straight chain alkylene group comprising 5, 6, or 7 carbon atoms, wherein one CH₂ group may be replaced by an oxygen or a sulfur atom, or wherein 2 carbon atoms form a C═C double bond, and which is either unsubstituted or substituted by one or two substituents selected from (1-4C)alkyl and halogen atoms, their enantiomers, diastereoisomers, racemates and mixtures thereof and pharmaceutically acceptable salts.

In one aspect, the inhibitor of histone deacetylase activity may be a carbamic acid compound, such as, for example, compounds disclosed in WO2002026696, having the general formula:

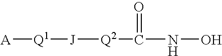

Wherein A is an aryl group; Q¹ is an aryl leader group having a backbone of at least 2 carbon atoms; J is an amide linkage selected from:

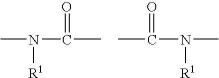

R₁ is an amido substituent; and, Q² is an acid leader group; and wherein: A, is a C₅₋₂₀aryl group, and is optionally substituted; the aryl leader group is a C₁₋₇alkylene group and is optionally substituted; the amido substituent, R¹, is hydrogen, C₁₋₇alkyl, C₃₋₂₀heterocyclyl, or C₅₋₂₀aryl; the acid leader group, Q², is C₁₋₇alkylene; C₅₋₂₀arylene; C₅₋₂₀arylene-C₁₋₇alkylene; C₁₋₇alkylene-C₅₋₂₀arylene; and is optionally substituted; and, the acid leader group, Q², has a backbone of at least 3 carbon atoms; and pharmaceutically. acceptable salts, solvates, amides, esters, ethers, chemically protected forms, and prodrugs thereof.

In one aspect, the inhibitor of histone deacetylase activity may be a dioxane compound, such as, for example, compounds disclosed in WO2002089782, having the general formula:

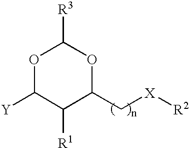

Wherein R¹ is hydrogen, or an aliphatic, heteroaliphatic, aryl, heteroaryl, -(aliphatic)aryl, -(aliphatic)heteroaryl, -(heteroaliphatic)aryl, or -(heteroaliphatic)heteroaryl moiety; n is 1-5; R² is hydrogen, a protecting group, or an aliphatic, heteroaliphatic, aryl, heteroaryl, -(aliphatic)aryl, -(aliphatic)heteroaryl, -(heteroaliphatic)aryl, or -(heteroaliphatic)heteroaryl moiety; X is —O—, —C(R^{2A})₂—, —S—, or —NR^{2A}—, wherein R^{2A} is hydrogen, a protecting group, or an aliphatic, heteroaliphatic, aryl, heteroaryl, -(aliphatic)aryl, -(aliphatic)heteroaryl, (heteroaliphatic)aryl, or -(heteroaliphatic)heteroaryl moiety; or wherein two or more occurrences of R² and R^{2A}, taken together, form a cyclic aliphatic or heteroaliphatic moiety, or an aryl or heteroaryl moiety; R³ is an aliphatic, heteroaliphatic, aryl, heteroaryl, -(aliphatic)aryl, -(aliphatic)heteroaryl, -(heteroaliphatic)aryl, or -(heteroaliphatic)heteroaryl moiety; and Y is hydrogen or an aliphatic, heteroaliphatic, aryl, heteroaryl, -(aliphatic)aryl, (aliphatic)heteroaryl, -(heteroaliphatic)aryl, or -(heteroaliphatic)heteroaryl moiety, whereby each of the foregoing aliphatic and heteroaliphatic groups is independently substituted or unsubstituted, cyclic or acyclic, linear or branched, and each of the foregoing aryl and heteroaryl groups is substituted or unsubstituted.

In one aspect, the inhibitor of histone deacetylase activity may be a compound having the general formula:

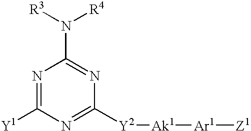

Wherein R³ and R⁴ are independently selected from the group consisting of hydrogen, L¹, Cy¹, and -L¹-Cy¹, wherein L¹ is C₁-C₆ alkyl, C₂-C₆ heteroalkyl, or C₃-C₆ alkenyl; and Cy¹ is cycloalkyl, aryl, heteroaryl, or heterocyclyl, each of which is optionally substituted, and each of which is optionally fused to one or two aryl or heteroaryl rings, or to one or two saturated or partially unsaturated cycloalkyl or heterocyclic rings, each of which rings is optionally substituted; or R³ and R⁴ are taken together with the adjacent nitrogen atom to form a 5-, 6-, or 7- membered ring, wherein the ring atoms are independently selected from the group consisting of C, O. S. and N. and wherein the ring is optionally substituted, and optionally forms part of a bicyclic ring system, or is optionally fused to one or two aryl or heteroaryl rings, or to one or two saturated or partially unsaturated cycloalkyl or heterocyclic rings, each of which rings and ring systems is optionally substituted; Y¹ is selected from the group consisting of —N(R¹)(R²), —CH2-C(O)—N(R¹)(R²), halogen, and hydrogen, wherein R and R are independently selected from the group consisting of hydrogen, L¹, Cy¹, and -L¹-Cy¹, wherein L¹ is C₁-C₆ alkyl, C₂-C₆ heteroalkyl, or C₃-C₆ alkenyl; and Cy¹ is cycloalkyl, aryl, heteroaryl, or heterocyclyl, each of which is optionally substituted, and each of which is optionally fused to one or two aryl or heteroaryl rings, or to one or two saturated or partially unsaturated cycloalkyl or heterocyclic rings, each of which rings is optionally substituted; or R¹ and R² are taken together with the adjacent nitrogen atom to form a 5-, 6-, or 7-membered ring, wherein the ring atoms are independently selected from the group consisting of C, O. S. and N. and wherein the ring is optionally substituted, and optionally forms part of a bicyclic ring system, or is optionally fused to one or two aryl or heteroaryl rings, or to one or two saturated or partially unsaturated cycloalkyl or heterocyclic rings, each of which rings and ring systems is optionally substituted; Y² is a chemical bond or N(R⁰), where R⁰ is selected from the group consisting of hydrogen, alkyl, aryl, aralkyl, and acyl; Ak¹ is C₁-C₆ alkylene, C₁-C₆-heteroalkylene (preferably, in which one (H₂— is replaced with —NH—, and more preferably —NH—CH₂—), C₂-C₆ alkenylene or C₂-C₆ alkynylene; Ar¹ is arylene or heteroarylene, either of which is optionally substituted; and Z¹ is selected from the group consisting of

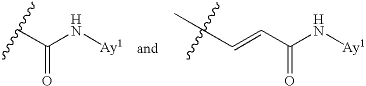

wherein Ay¹ is aryl or heteroaryl, each of which is optionally substituted. Such compounds are disclosed in WO2003024448.

In one aspect, the inhibitor of histone deacetylase activity may be a carbamic acid compound, such as, for example, compounds disclosed in WO2002030879, having the general formula:

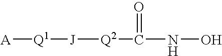

Wherein A is an aryl group; Q¹ is a covalent bond or an aryl leader group; J is a sulfonamide linkage selected from:

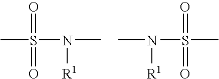

R¹ is a sulfonamido substituent; and, Q² is an acid leader group; with the proviso that if J is:

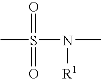

then Q¹ is an aryl leader group; and wherein: A, is a C₅₋₂₀aryl group, and is optionally substituted; the aryl leader group, if present, is a C₁₋₇alkylene group and is optionally substituted; the sulfonamido substituent, R¹, is hydrogen, C₁₋₇alkyl, C₃₋₂₀heterocyclyl, or C₅₋₂₀aryl; the acid leader group, Q², is C₁₋₇alkylene; C₅₋₂₀arylene; C₅₋₂₀arylene-C₁₋₇alkylene; C₁₋₇alkylene-C₅₋₂₀arylene; or an ether linkage; and is optionally substituted; and pharmaceutically acceptable salts, solvates, amides, esters, ethers, chemically protected forms, and prodrugs thereof.

In one aspect, the inhibitor of histone deacetylase activity may be a carbamic acid compound, such as, for example, compounds disclosed in WO2003082288, having the general formula:

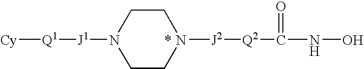

Wherein Cy is independently a cyclyl group; Q¹ is independently a covalent bond or cyclyl leader group; the piperazin-1,4-diyl group is optionally substituted; J¹ is independently a covalent bond or —C(═O)—; J² is independently —C(═O)— or —S(═O)₂—; Q² is independently an acid leader group; wherein: Cy is independently: C₃₋₂₀carbocyclyl, C₃₋₂₀heterocyclyl, or C₅₋₂₀aryl; and is optionally substituted; Q¹ is independently: a covalent bond; C₁₋₇alkylene; or C₁₋₇alkylene-X—C₁₋₇alkylene, —X—C₁₋₇alkylene, or C₁₋₇alkylene-X—, wherein X is —O— or —S—; and is optionally substituted; Q² is independently: C₄₋₈alkylene; and is optionally substituted; and has a backbone length of at least 4 atoms; or: Q² is independently: C₅₋₂₀arylene; C₅₋₂₀arylene-C₁₋₇alkylene; C₁₋₇alkylene-C₅₋₂₀arylene; or, C₁₋₇alkylene-C₅₋₂₀arylene-C₁₋₇alkylene; and is optionally substituted; and has a backbone length of at least 4 atoms; or a pharmaceutically acceptable salt, solvate, amide, ester, ether, chemically protected form, or prodrug thereof.

In one aspect, the inhibitor of histone deacetylase activity may be piperazinyl-, piperidinyl- and morpholinyl-derivatives, such as, for example, compounds disclosed in WO2003076438, having the general formula:

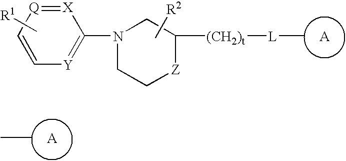

the N-oxide forms, the pharmaceutically acceptable addition salts and the stereo chemically isomeric forms thereof, wherein t is 0, 1, 2, 3 or 4 and when t is 0 then a direct bond is intended;
each Q is nitrogen or

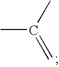

each X is nitrogen or

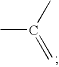

each Y is nitrogen or

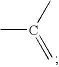

each Z is —NH—, —O— or —CH₂₋;

R¹ is —C(O)NR³R⁴, —NHC(O)R⁷, —C(O)—C₁₋₆alkanediylSR⁷, —NR⁸C(O)N(OH)R⁷, —NR⁸C(O)C₁₋₆alkanediylSR⁷, —NR⁸C(O)C═N(OH)R⁷ or another Zn-chelating-group wherein R³ and R⁴ are each independently selected from hydrogen, hydroxy, C₁₋₆alkyl, hydroxyC₁₋₆alkyl, aminoC₁₋₆alkyl or aminoaryl; R⁷ is hydrogen, C₁₋₆alkyl, C₁₋₆alkylcarbonyl, arylC₁₋₆alkyl, C₁₋₆alkylpyrazinyl, pyridinone, pyrrolidinone or methylimidazolyl; R⁸ is hydrogen or C₁₋₆alkyl; R² is hydrogen, hydroxy, amino, hydroxyC₁₋₆alkyl, C₁₋₆alkyl, C₁₋₆alkyloxy, arylC₁₋₆alkyl, aminocarbonyl, hydroxycarbonyl, aminoC₁₋₆alkyl, aminocarbonylC₁₋₆alkyl, hydroxycarbonylC₁₋₆alkyl, hydroxyaminocarbonyl, C₁₋₆alkyloxycarbonyl, C₁₋₆alkylaminoC₁₋₆alkyl or di(C₁₋₆alkyl)aminoC₁₋₆ alkyl; -L- is a bivalent radical selected from —NR⁹C(O)—, —NR⁹SO₂— or —NR⁹CH₂— wherein R⁹ is hydrogen, C₁₋₆alkyl, C₃₋₁₀cycloalkyl, hydroxyC₁₋₆alkyl, C₁₋6alkyloxyC₁₋₆alkyl or di(C₁₋₆alkyl)aminoC₁₋₆alkyl;

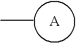

is a radical selected from

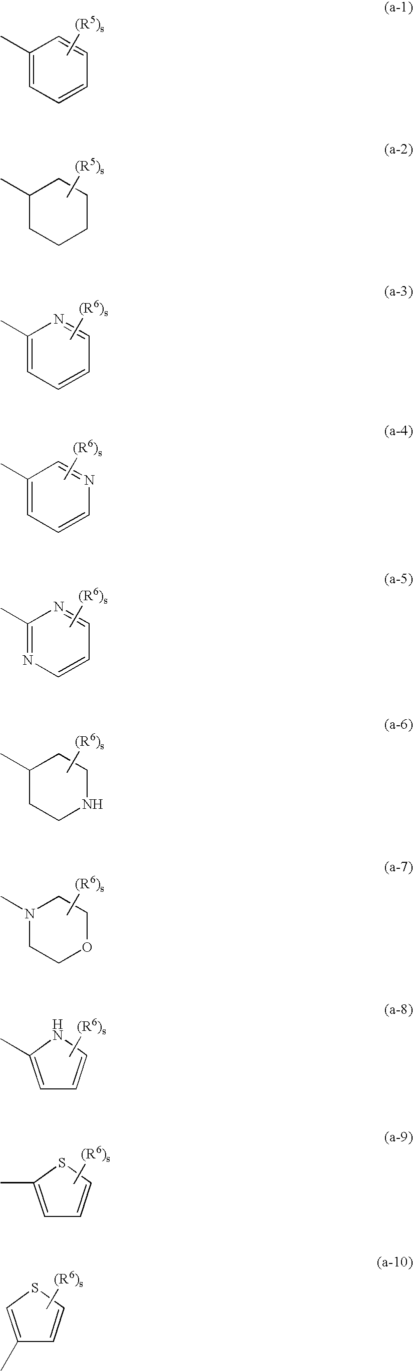

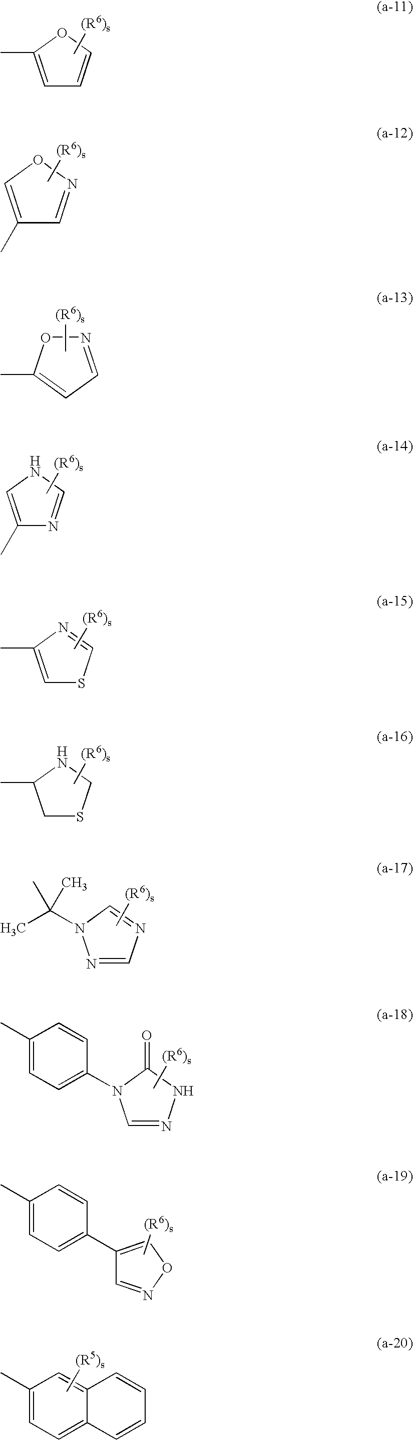

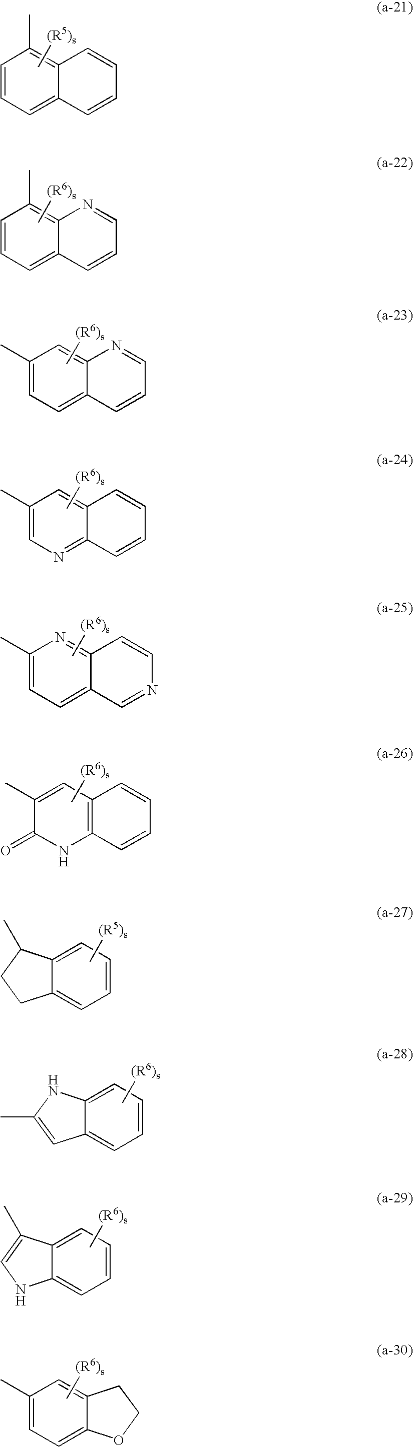

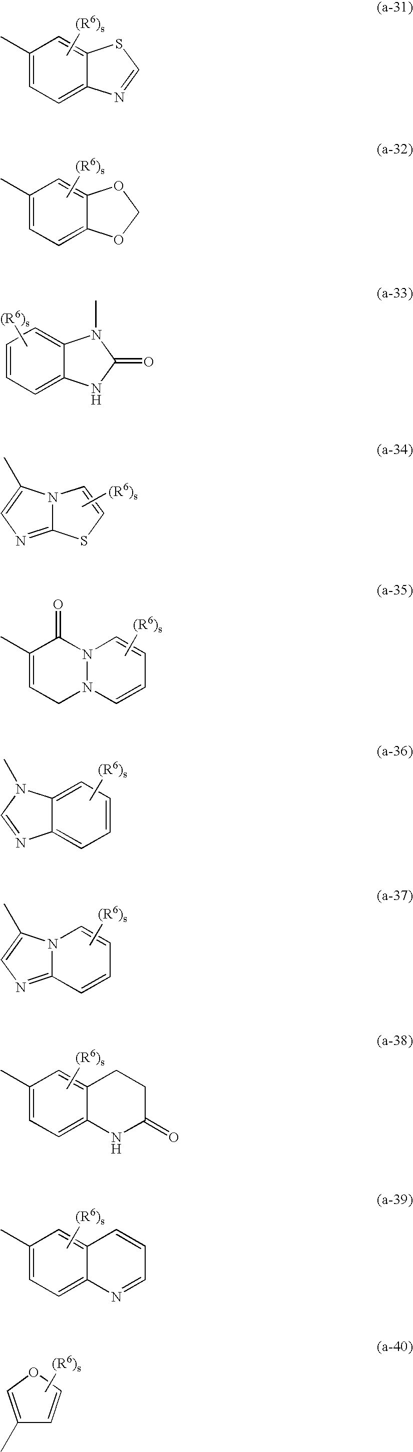

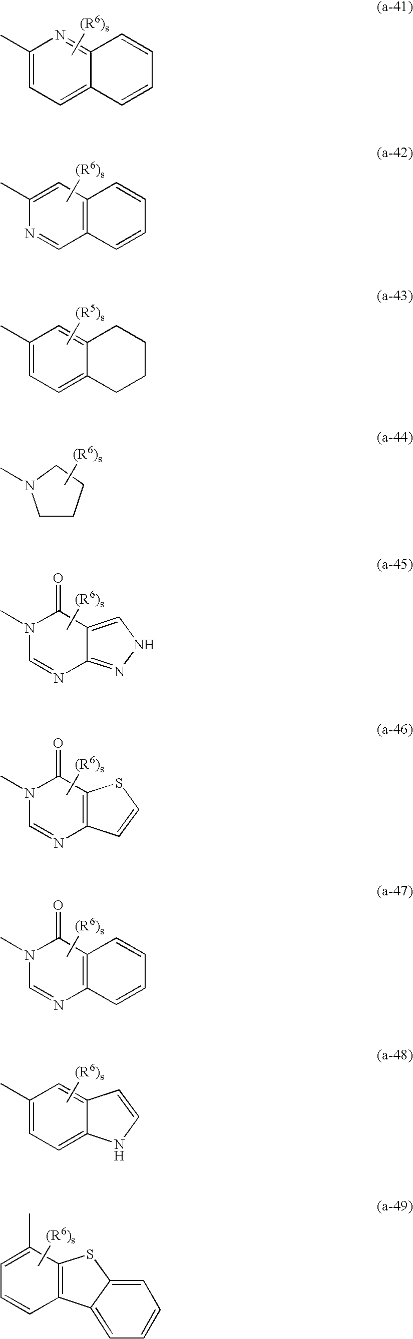

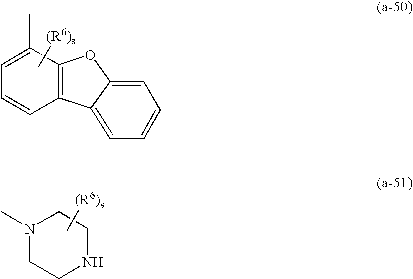

wherein each s is independently 0, 1, 2, 3, 4 or 5; each R⁵ and R⁶ are independently selected from hydrogen; halo; hydroxy; amino; nitro; trihaloC₁₋₆alkyl; trihaloC₁₋₆alkyloxy; C₁₋₆alkyl; C₁₋₆alkyl substituted with aryl and C₃₋₁₀cycloalkyl; C₁₋₆alkyloxy; C₁₋₆alkyloxyC₁₋₆alkyloxy; C₁₋₆alkylcarbonyl; C₁₋₆alkyloxycarbonyl; C₁₋₆alkylsulfonyl; cyanoC₁₋₆alkyl; hydroxyC₁₋₆alkyl; hydroxyC₁₋₆alkyloxy; hydroxyC₁₋₆alkylamino; aminoC₁₋₆alkyloxy; di(C₁₋₆alkyl)aminocarbonyl; di(hydroxyC₁₋₆alkyl)amino; (aryl)(C₁₋₆alkyl)amino; di(C₁₋₆alkyl)amino C₁₋₆alkyloxy; di(C₁₋₆alkyl)amino C₁₋₆alkylamino; di(C₁₋₆alkyl)amino C₁₋₆alkylamino C₁₋₆alkyl; arylsulfonyl; arylsulfonylamino; aryloxy; aryloxy C₁₋₆alkyl; arylC_{2 6}alkenediyl; di(C₁₋₆alkyl)amino; di(C₁₋₆alkyl)amino C₁₋₆alkyl; di(C₁₋₆alkyl)amino(C₁₋₆alkyl)amino; di(C₁₋₆alkyl)amino(C₁₋₆alkyl)amino C₁₋₆alkyl; di(C₁₋₆alkyl)amino C₁₋₆alkyl(C₁₋₆alkyl)amino; di(C₁₋₆alkyl)aminoC₁₋₆alkyl(C₁₋₆alkyl)amino C₁₋₆alkyl; aminosulfonylamino(C₁₋₆alkyl)amino; aminosulfonylamino(C₁₋₆alkyl)amino C₁₋₆alkyl; di(C₁₋₆alkyl)aminosulfonylamino(C₁₋₆alkyl)amino; di(C₁₋₆alkyl)aminosulfonylamino(C₁₋₆alkyl)amino C₁₋₆alkyl; cyano; thiophenyl; thiophenyl substituted with di(C₁₋₆alkyl)amino C₁₋₆alkyl(C₁₋₆alkyl)amino C₁₋₆alkyl, di(C₁₋₆alkyl)amino C₁₋₆alkyl, C₁₋₆alkylpiperazinyl C₁₋₆alkyl, hydroxy C₁₋₆alkylpiperazinyl C₁₋₆alkyl, hydroxy C₁₋₆alkyloxy C₁₋₆alkylpiperazinyl C₁₋₆alkyl, di(C₁₋₆alkyl)aminosulfonylpiperazinyl C₁₋₆alkyl, C₁₋₆alkyloxypiperidinyl, C₁₋₆alkyloxypiperidinyl C₁₋₆alkyl, morpholinyl C₁₋₆alkyl, hydroxy C₁₋₆alkyl(C₁₋₆alkyl)amino C₁₋₆alkyl, or di(hydroxy C₁₋₆alkyl)amino C₁₋₆alkyl; furanyl; furanyl substituted with hydroxy C₁₋₆alkyl; benzofuranyl; imidazolyl; oxazolyl; oxazolyl substituted with aryl and C₁₋₆alkyl; C₁₋₆alkyltriazolyl; tetrazolyl; pyrrolidinyl; pyrrolyl; piperidinyl C₁₋₆alkyloxy; morpholinyl; C₁₋₆alkylmorpholinyl; morpholinyl C₁₋₆alkyloxy; morpholinyl C₁₋₆alkyl; morpholinyl C₁₋₆alkylamino; morpholinylC₁₋₆alkylamino C₁₋₆alkyl; piperazinyl; C₁₋₆alkylpiperazinyl; C₁₋₆alkylpiperazinyl C₁₋₆alkyloxy, piperazinyl C₁₋₆alkyl; naphtalenylsulfonylpiperazinyl; naphtalenylsulfonylpiperidinyl; naphtalenylsulfonyl; C₁₋₆alkylpiperazinyl C₁₋₆alkyl; C₁₋₆alkylpiperazinyl C₁₋₆alkylamino; C₁₋₆alkylpiperazinyl C₁₋₆alkylamino C₁₋₆alkyl; C₁₋₆alkylpiperazinylsulfonyl; aminosulfonylpiperazinyl C₁₋₆alkyloxy; aminosulfonylpiperazinyl; aminosulfonylpiperazinyl C₁₋₆alkyl; di(C₁₋₆alkyl)aminosulfonylpiperazinyl; di(C₁₋₆alkyl)aminosulfonylpiperazinyl C₁₋₆alkyl; hydroxy C₁₋₆alkylpiperazinyl; hydroxy C₁₋₆alkylpiperazinyl C₁₋₆alkyl; C₁₋₆alkyloxypiperidinyl; C₁₋₆alkyloxypiperidinyl C₁₋₆alkyl; piperidinylamino C₁₋₆alkylamino; piperidinylamino C₁₋₆alkylaminoC₁₋₆alkyl; (C₁₋₆alkyIpiperidillyl)(hydroxyC₁₋₆alkyl)amino C₁₋₆alkylamino; (C₁₋₆alkylpiperidinyl)(hydroxy C₁₋₆alkyl)amino C₁₋₆alkylamino C₁₋₆alkyl; hydroxy C₁₋₆alkyloxy C₁₋₆alkylpiperazinyl; hydroxy C₁₋₆alkyloxy C₁₋₆alkylpiperazinyl C₁₋₆alkyl; (hydroxy C₁₋₆alkyl)(C₁₋₆alkyl)amino; (hydroxy C₁₋₆alkyl)(C₁₋₆alkyl)aminoC₁₋₆alkyl; hydroxy C₁₋₆alkylamino C₁₋₆alkyl; di(hydroxy C₁₋₆alkyl)amino C₁₋₆alkyl; pyrrolidinyl C₁₋₆alkyl; pyrrolidinylC₁₋₆alkyloxy; pyrazolyl; thiopyrazolyl; pyrazolyl substituted with two substituents selected from C₁₋₆alkyl or trihaloC₁₋₆alkyl; pyridinyl; pyridinyl substituted with C₁₋₆alkyloxy, aryloxy or aryl; pyrimidinyl; tetrahydropyrimidinylpiperazinyl; tetrahydropyrimidinylpiperazinylC₁₋₆alkyl; quinolinyl; indole; phenyl; phenyl substituted with one, two or three substituents independently selected from halo, amino, nitro, C₁₋₆alkyl, C₁₋₆alkyloxy, hydroxyC₁₋₄alkyl, trifluoromethyl, trifluoromethyloxy, hydroxyC₁₋₄alkyloxy, C₁₋₄alkylsulfonyl, C₁₋₄alkyloxyC₁₋₄alkyloxy, C₁₋₄alkyloxycarbonyl, amino C₁₋₄alkyloxy, di(C₁₋₄alkyl)amino C₁₋₄alkyloxy, di(C₁₋₄alkyl)amino, di(C₁₋₄alkyl)aminocarbonyl, di(C₁₋₄alkyl)amino C₁₋₄alkyl, di(C₁₋₄alkyl)aminoC₁₋₄alkylamino C₁₋₄alkyl, di(C₁₋₄alkyl)amino(C₁₋₄alkyl)amino, di(C₁₋₄alkyl)amino(C₁₋₄alkyl)aminoC₁₋₄alkyl, di(C₁₋₄alkyl)amino C₁₋₄alkyl(C₁₋₄alkyl)amino, di(C₁₋₄alkyl)amino C₁₋₄alkyl(C₁₋₄alkyl)amino C₁₋₄alkyl, aminosulfonylamino(C₁₋₄alkyl)amino, aminosulfonylamino(C₁₋₄alkyl)amino C₁₋₄alkyl, di(C₁₋₄alkyl)aminosulfonyl amino(C₁₋₄alkyl)amino, di(C₁₋₄alkyl)aminosulfonylamino(C₁₋₄alkyl)amino C₁₋₆alkyl, cyano, piperidinyl C₁₋₄alkyloxy, pyrrolidinyl C₁₋₄alkyloxy, aminosulfonylpiperazinyl, aminosulfonylpiperazinyl C₁₋₄alkyl, di(C₁₋₄alkyl)aminosulfonylpiperazinyl, di(C₁₋₄alkyl)aminosulfonylpiperazinyl C₁₋₄alkyl, hydroxyC₁₋₄alkylpiperazinyl, hydroxy C₁₋₄alkylpiperazinyl C₁₋₄alkyl, C₁₋₄alkyloxypiperidinyl, C₁₋₄alkyloxypiperidinylC₁₋₄alkyl, hydroxy C₁₋₄alkyloxyC₁₋₄alkylpiperazinyl, hydroxyC₁₋₄alkyloxyC₁₋₄alkylpiperazinylC₁₋₄alkyl, (hydroxyC₁₋₄alkyl)(C₁₋₄alkyl)amino, (hydroxyC₁₋₄alkyl)(C₁₋₄alkyl)aminoC₁₋₄alkyl, hydroxyC₁₋₄alkylaminoC₁₋₄alkyl, di(hydroxyC₁₋₄alkyl)aminoC₁₋₄alkyl, furanyl, furanyl substituted with —CH═CH—CH═CH—, pyrrolidinylC₁₋₄alkyl, pyrrolidinylC₁₋₄alkyloxy, morpholinyl, morpholinylC₁₋₄alkyloxy, morpholinylC₁₋₄alkyl, morpholinylC₁₋₄alkylamino, morpholinylC₁₋₄alkylaminoC₁₋₄alkyl, piperazinyl, C₁₋₄alkylpiperazinyl, C₁₋₄alkylpiperazinylC₁₋₄alkyloxy, piperazinylC₁₋₄alkyl, C₁₋₄alkylpiperazinylC₁₋₄alkyl, C₁₋₄alkylpiperazinylC₁₋₄alkylamino, C₁₋₄alkylpiperazinylC₁₋₄alkylamino C₁₋₆alkyl, pyrimidinylpiperazinyl, pyrimidinylpiperazinylC₁₋₄alkyl, piperidinylaminoC₁₋₄alkyl amino, piperidinylaminoC₁₋₄alkylaminoC₁₋₄alkyl, (C₁₋₄alkylpiperidinyl)(hydroxyC₁₋₄alkyl)aminoC₁₋₄alkylamino, (C₁₋₄alkylpiperidinyl)(hydroxyC₁₋₄alkyl)aminoC₁₋₄alkylaminoC₁₋₄alkyl, pyridinylC₁₋₄alkyloxy, hydroxyC₁₋₄alkylamino, di(hydroxyC₁₋₄alkyl)amino, di(C₁₋₄alkyl)aminoC₁₋₄alkylamino, aminothiadiazolyl, aminosulfonylpiperazinylC₁₋₄alkyloxy, or thiophenylC₁₋₄alkylamino; each R⁵ and R⁶ can be placed on the nitrogen in replacement of the hydrogen; aryl in the above is phenyl, or phenyl substituted with one or more substituents each independently selected from halo, C₁₋₆alkyl, C₁₋₆alkyloxy, trifluoromethyl, cyano or hydroxycarbonyl.

In one aspect, the inhibitor of histone deacetylase activity may be a compound of the general formula:

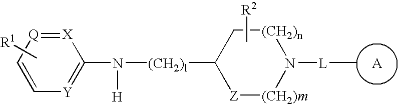

the N-oxide forms, the pharmaceutically acceptable addition salts and the stereo-chemically isomeric forms thereof, wherein n is 0, 1, 2 or 3 and when n is 0 then a direct bond is intended; m is 0, 1, 2 or 3 and when m is 0 then a direct bond is intended; t is 0 or 1 and when t is 0 then a direct bond is intended;
each Q is nitrogen or

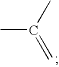

each X is nitrogen or

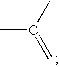

each Y is nitrogen or

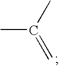

each Z is —CH₂— or —O—;

R¹ is —C(O)NR³R⁴, —N(H)C(O)R⁷, —C(O)—C₁₋₆alkanediylSR⁷, —NR⁸C(O)N(OH)R⁷, —NR⁸C(O)C₁₋₆alkanediylSR⁷, —NR³C(O)C═N(OH)R⁷ or another Zn-chelating-group wherein R³ and R⁴ are each independently selected from hydrogen, hydroxy, C₁₋₆alkyl, hydroxy C₁₋₆alkyl, amino C₁₋₆alkyl or aminoaryl; R⁷ is independently selected from hydrogen, C₁₋₆6alkyl, C₁₋₆alkylcarbonyl, arylC₁₋₆alkyl, C₁₋₆alkylpyrazinyl, pyridinone, pyrrolidinone or methylimidazolyl; R⁸ is independently selected from hydrogen or C₁₋₆alkyl; R² is hydrogen, hydroxy, amino, hydroxyC₁₋₆alkyl, C₁₋₆alkyl, C₁₋₆alkyloxy, arylC₁₋₆alkyl, aminocarbonyl, hydroxycarbonyl, aminoC₁₋₆alkyl, aminocarbonylC₁₋₆alkyl, hydroxycarbonylC₁₋₆alkyl, hydroxyaminocarbonyl, C₁₋₆alkyloxycarbonyl, C₁₋₆alkylaminoC₁₋₆alkyl or di(C₁₋₆alkyl)aminoC₁₋₆alkyl; -L- is a bivalent radical selected from C₁₋₆alkanediyl, carbonyl, sulfonyl, or C₁₋₆alkanediyl substituted with phenyl

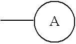

is a radical selected from

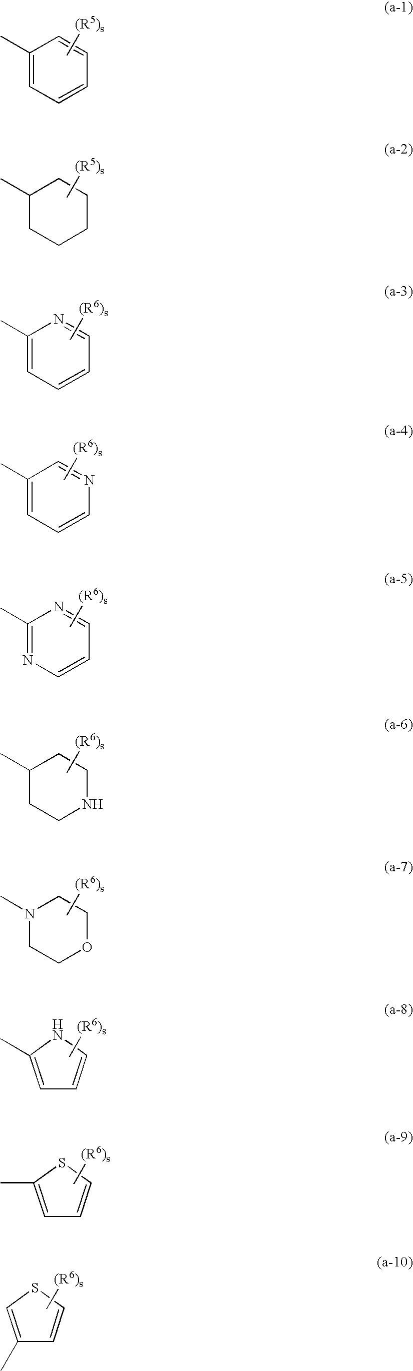

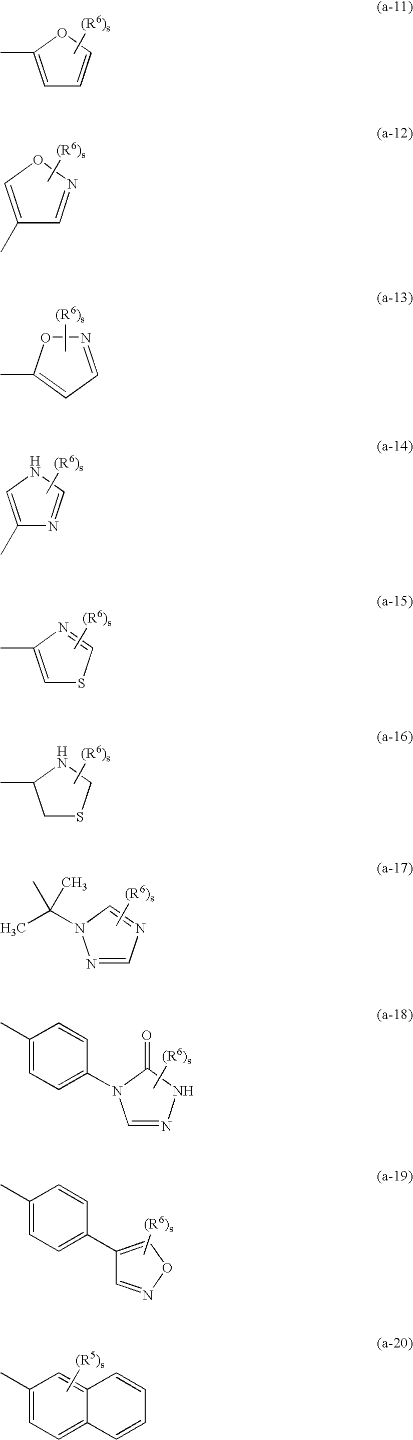

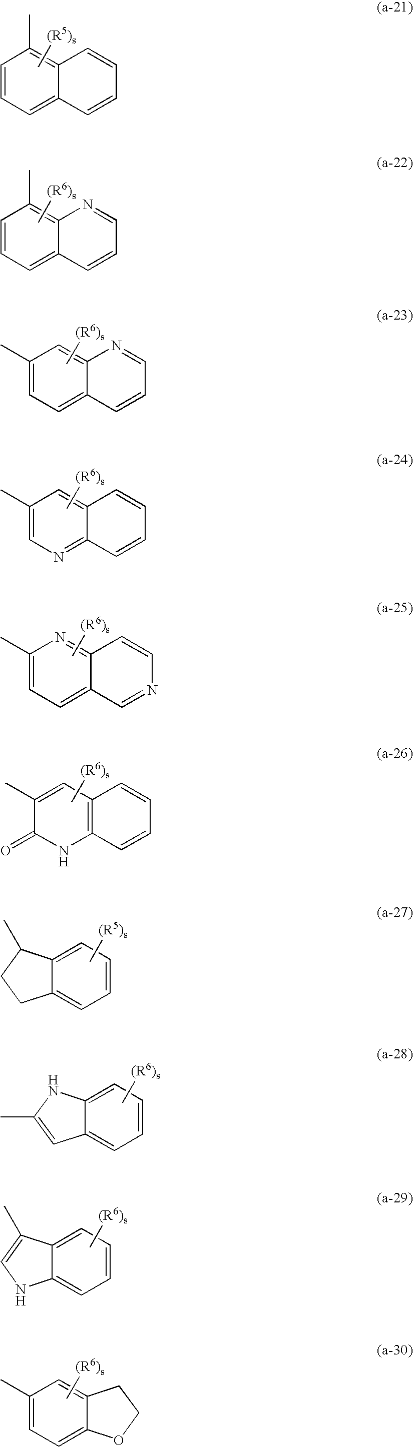

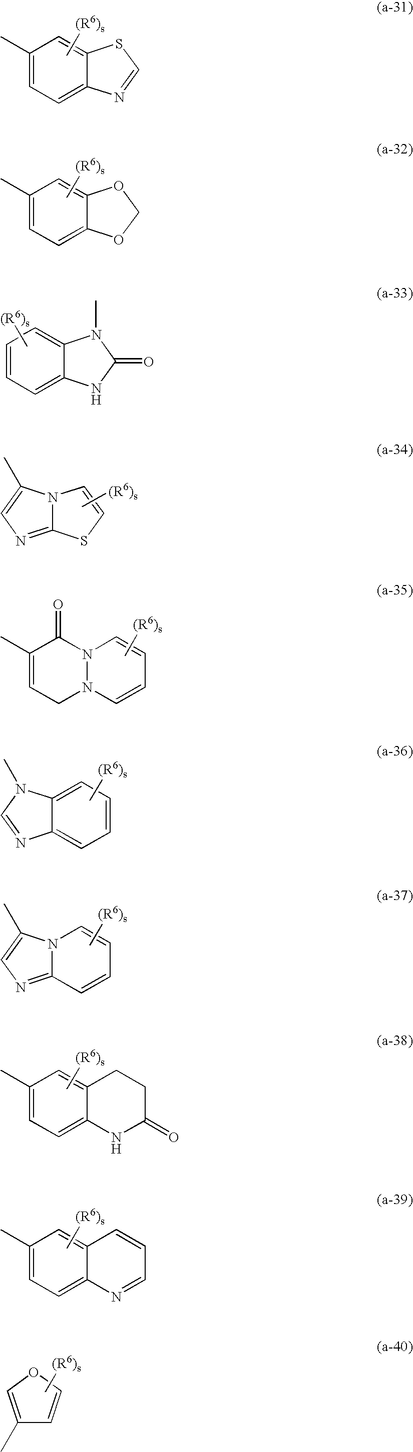

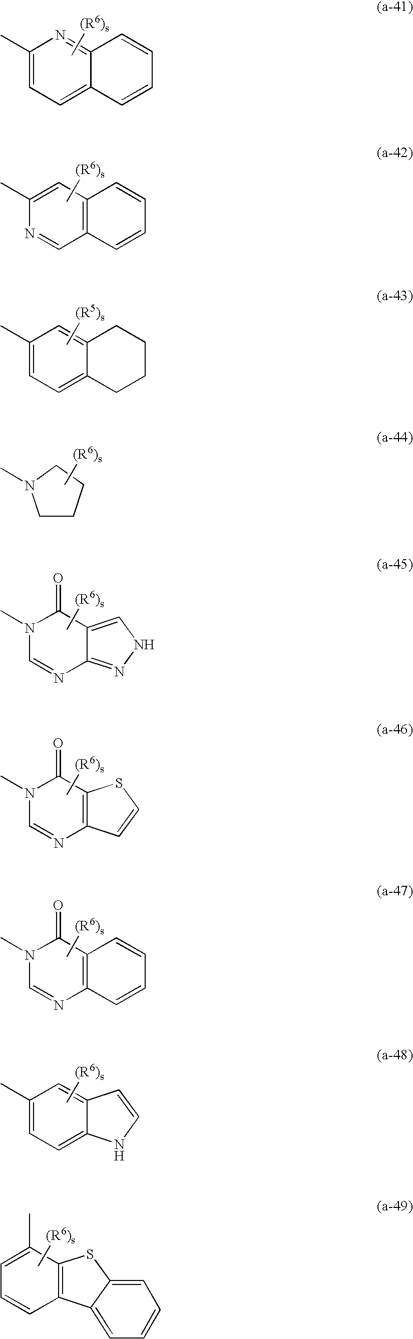

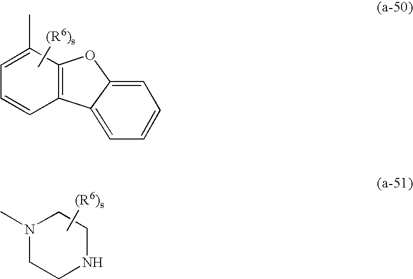

wherein each s is independently 0, 1, 2, 3, 4 or 5; each R⁵ and R⁶ are independently selected from hydrogen; halo; hydroxy; amino; nitro; trihaloC₁₋₆alkyl; trihaloC₁₋₆alkyloxy; C₁₋₆alkyl; C₁₋₆alkyl substituted with aryl and C₃₋₁₀cycloalkyl; C₁₋₆alkyloxy; C₁₋₆alkyloxyC₁₋₆alkyloxy; C₁₋₆alkylcarbonyl; C₁₋₆alkyloxycarbonyl; C₁₋₆alkylsulfonyl; cyanoC₁₋₆alkyl; hydroxyC₁₋₆alkyl; hydroxyC₁₋₆alkyloxy; hydroxyC₁₋₆alkylamino; aminoC₁₋₆alkyloxy; di(C₁₋₆alkyl)aminocarbonyl; di(hydroxyC₁₋₆alkyl)amino; (aryl)(C₁₋₆alkyl)amino; di(C₁₋₆alkyl)aminoC₁₋₆alkyloxy; di(C₁₋₆alkyl)aminoC₁₋₆alkylamino; di(C₁₋₆alkyl)aminoC₁₋₆alkylaminoC₁₋₆alkyl; arylsulfonyl; arylsulfonylamino; aryloxy; aryloxyC₁₋₆alkyl; arylC₂₋₆alkenediyl; di(C₁₋₆alkyl)amino; di(C₁₋₆alkyl)aminoC₁₋₆alkyl; di(C₁₋₆alkyl)amino(C₁₋₆alkyl)amino; di(C₁₋₆alkyl)amino(C₁₋₆alkyl)amino C₁₋₆alkyl; di(C₁₋₆alkyl)amino C₁₋₆alkyl (C₁₋₆alkyl)amino; di(C₁₋₆alkyl)amino C₁₋₆alkyl(C₁₋₆alkyl)amino C₁₋₆alkyl; aminosulfonylamino(C₁₋₆alkyl)amino; aminosulfonylamino(C₁₋₆alkyl)amino C₁₋₆alkyl; di(C₁₋₆alkyl)aminosulfonylamino(C₁₋₆alkyl)amino; di(C₁₋₆alkyl)aminosulfonylamino(C₁₋₆alkyl)amino C₁₋₆alkyl; cyano; thiophenyl; thiophenyl substituted with di(C₁₋₆alkyl)amino C₁₋₆alkyl(C₁₋₆alkyl)amino C₁₋₆alkyl, di(C₁₋₆alkyl)amino C₁₋₆alkyl, C₁₋₆alkylpiperazinylC₁₋₆alkyl, hydroxyC₁₋₆alkylpiperazinylC₁₋₆alkyl, hydroxy C₁₋₆alkyloxyC₁₋₆alkylpiperazinylC₁₋₆alkyl, di(C₁₋₆alkyl)aminosulfonylpiperazinylC₁₋₆alkyl, C₁₋₆alkyloxypiperidinyl, C₁₋₆alkyloxypiperidinylC₁₋₆alkyl, morpholinylC₁₋₆alkyl, hydroxyC₁₋₆alkyl(C₁₋₆alkyl)aminoC₁₋₆alkyl, or di(hydroxyC₁₋₆alkyl)aminoC₁₋₆alkyl; furanyl; furanyl substituted with hydroxyC₁₋₆alkyl; benzofuranyl; imidazolyl; oxazolyl; oxazolyl substituted with aryl and C₁₋₆alkyl; C₁₋₆alkyltriazolyl; tetrazolyl; pyrrolidinyl; pyrrolyl; piperidinylC₁₋₆alkyloxy; morpholinyl; C₁₋₆alkylmorpholinyl; morpholinylC₁₋₆alkyloxy; morpholinylC₁₋₆alkyl; morpholinylC₁₋₆alkylamino; morpholinylC₁₋₆alkylaminoC₁₋₆alkyl; piperazinyl; C₁₋₆alkylpiperazinyl; C₁₋₆alkylpiperazinylC₁₋₆alkyloxy; piperazinylC₁₋₆alkyl; naphtalenylsulfonylpiperazinyl; naphtalenylsulfonylpiperidinyl; naphtalenylsulfonyl: C₁₋₆alkylpiperazinylC₁₋₆alkyl; C₁₋₆alkylpiperazinylC₁₋₆alkylamino; C₁₋₆alkylpiperazinylC₁₋₆alkylaminoC₁₋₆alkyl; C₁₋₆alkylpiperazinylsulfonyl; aminosulfonylpiperazinylC₁₋₆alkyloxy; aminosulfonylpiperazinyl; aminosulfonylpiperazinylC₁₋₆alkyl; di(C₁₋₆alkyl)aminosulfonylpiperazinyl; di(C₁₋₆alkyl)aminosulfonylpiperazinylC₁₋₆alkyl; hydroxyC₁₋₆alkylpiperazinyl; hydroxycC₁₋₆alkylpiperazinylC₁₋₆alkyl; C₁₋₆alkyloxypiperidinyl; C₁₋₆alkyloxypiperidinylC₁₋₆alkyl; piperidinylaminoC₁₋₆alkylamino; piperidinylaminoC₁₋₆alkylaminoC₁₋₆alkyl; (C₁₋₆alkylpiperidinyl)(hydroxyC₁₋₆alkyl)aminoC₁₋₆alkylamino; (C₁₋₆alkylpiperidinyl)(hydroxyC₁₋₆alkyl)aminoC₁₋₆alkylaminoC₁₋₆alkyl; hydroxyC₁₋₆alkyloxyC₁₋₆alkylpiperazinyl; hydroxyC₁₋₆alkyloxyC₁₋₆alkylpiperazinyl C₁₋₆alkyl; (hydroxyC₁₋₆alkyl)(C₁₋₆alkyl)amino; (hydroxyC₁₋₆alkyl)(C₁₋₆alkyl)aminoC₁₋₆alkyl; hydroxyC₁₋₆alkylamino C₁₋₆alkyl; di(hydroxyC₁₋₆alkyl)aminoC₁₋₆alkyl; pyrrolidinylC₁₋₆alkyl; pyrrolidinylC₁₋₆alkyloxy; pyrazolyl; thiopyrazolyl; pyrazolyl substituted with two substituents selected from C₁₋₆alkyl or trihaloC₁₋₆alkyl; pyridinyl; pyridinyl substituted with C₁₋₆alkyloxy, aryloxy or aryl; pyrimidinyl; tetrahydropyrimidinylpiperazinyl; tetrahydropyrimidinylpiperazinylC₁₋₆alkyl; quinolinyl; indolyl; phenyl; phenyl substituted with one, two or three substituents independently selected from halo, amino, nitro, C₁₋₆alkyl, C₁₋₆alkyloxy, hydroxyC₁₋₄alkyl, trifluoromethyl, trifluoromethyloxy, hydroxyC₁₋₄alkyloxy, C₁₋₄alkylsulfonyl, C₁₋₄alkyloxyC₁₋₄alkyloxy, C₁₋₄alkyloxycarbonyl, aminoC₁₋₄alkyloxy, di(C₁₋₄alkyl)aminoC₁₋₄alkyloxy, di(C₁₋₄alkyl)amino, di(C₁₋₄alkyl)aminocarbonyl, di(C₁₋₄alkyl)aminoC₁₋₄alkyl, di(C₁₋₄alkyl)aminoC₁₋₄alkylaminoC₁₋₄alkyl, di(C₁₋₄alkyl)amino(C₁₋₄alkyl)amino, di(C₁₋₄alkyl)amino(C₁₋₄alkyl)aminoC₁₋₄alkyl, di(C₁₋₄alkyl)aminoC₁₋₄alkyl(C₁₋₄alkyl)amino, di(C₁₋₄alkyl)aminoC₁₋₄alkyl(C₁₋₄alkyl)aminoC₁₋₄alkyl, aminosulfonylamino(C₁₋₄alkyl)amino, aminosulfonylamino(C₁₋₄alkyl)aminoC₁₋₄alkyl, di(C₁₋₄alkyl)aminosulfonylamino(C₁₋₄alkyl)amino, di(C₁₋₄alkyl)aminosulfonylamino(C₁₋₄alkyl)amino C₁₋₆alkyl, cyano, piperidinylC₁₋₄alkyloxy, pyrrolidinylC₁₋₄alkyloxy, aminosulfonylpiperazinyl, aminosulfonylpiperazinylC₁₋₄alkyl, di(C₁₋₄alkyl)aminosulfonylpiperazinyl, di(C₁₋₄alkyl)aminosulfonylpiperazinylC₁₋₄alkyl, hydroxyC₁₋₄alkylpiperazinyl, hydroxyC₁₋₄alkylpiperazinyl C₁₋₄alkyl, C₁₋₄alkyloxypiperidinyl, C₁₋₄alkyloxypiperidinylC₁₋₄alkyl, hydroxyC₁₋₄alkyloxyC₁₋₄alkylpiperazinyl, hydroxyC₁₋₄alkyloxyC₁₋₄alkylpiperazinylC₁₋₄alkyl, (hydroxyC₁₋₄alkyl)(C₁₋₄alkyl)amino, (hydroxyC₁₋₄alkyl)(C₁₋₄alkyl)aminoC₁₋₄alkyl, di(hydroxyC₁₋₄alkyl)amino, di(hydroxyC₁₋₄alkyl)aminoC₁₋₄alkyl, furanyl, furanyl substituted with —CH═CH—CH═CH—, pyrrolidinylC₁₋₄alkyl, pyrrolidinylC₁₋₄alkyloxy, morpholinyl, morpholinylC₁₋₄alkyloxy, morpholinylC₁₋₄alkyl, morpholinylC₁₋₄alkylamino, morpholinylC₁₋₄alkylaminoC₁₋₄alkyl, piperazinyl, C₁₋₄alkylpiperazinyl, C₁₋₄alkylpiperazinylC₁₋₄alkyloxy, piperazinylC₁₋₄alkyl, C₁₋₄alkylpiperazinylC₁₋₄alkyl, C₁₋₄alkylpiperazinylC₁₋₄alkylamino, C₁₋₄alkylpiperazinylC₁₋₄alkylaminoC₁₋₆alkyl, tetrahydropyrimidinylpiperazinyl, tetrahydropyrimidinylpiperazinylC₁₋₄alky, piperidinylaminoC₁₋₄alkylamino, piperidinylaminoC₁₋₄alkylaminoCI ₄alkyl, (C₁₋₄alkylpiperidinyl)(hydroxyC₁₋₄alkyl)aminoC₁₋₄alkylamino, (C₁₋₄alkylpiperidinyl)(hydroxyC₁₋₄alkyl)aminoC₁₋₄alkylaminoC₁₋₄alkyl, pyridinylC₁₋₄alkyloxy, hydroxyC₁₋₄alkylamino, hydroxyC₁₋₄alkylaminoC₁₋₄alkyl, di(C₁₋₄alkyl)aminoC₁₋₄alkylamino, aminothiadiazolyl, aminosulfonylpiperazinylC₁₋₄alkyloxy, or thiophenylC₁₋₄alkylamino; each R⁵ and R⁶ can be placed on the nitrogen in replacement of the hydrogen; aryl in the above is phenyl, or phenyl substituted with one or more substituents each independently selected from halo, C₁₋₆alkyl, C₁₋₆alkyloxy, trifluoromethyl, cyano or hydroxycarbonyl. See, for example, compounds disclosed in WO2003076430.

In one aspect, the inhibitor of histone deacetylase activity may be a compound of the general formula:

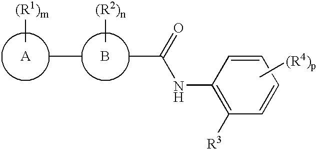

wherein: Ring A is a heterocyclyl, wherein if said heterocyclyl contains an —NH— moiety that nitrogen may be optionally substituted by a group selected from G; R¹ is a substituent on carbon and is selected from halo, nitro, cyano, hydroxy, oxo, trifluoromethyl, trifluoromethoxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂amino, C₁₋₆alkanoylamino, N—(C₁₋₆alkyl)carbamoyl, N,N—(C₁₋₆alkyl)₂carbamoyl, C₁₋₆alkylS(O)ₐ wherein a is 0 to 2, C₁₋₆alkoxycarbonyl, N—(C₁₋₆alkyl)sulphamoyl, N,N—(C₁₋₆alkyl)₂sulphamoyl, aryl, aryloxy, arylC₁₋₆alkyl, heterocyclic group, (heterocyclic group)C₁₋₆alkyl or a group (D-E-); wherein R¹, including group (D-E-), may be optionally substituted on carbon by one or more V; and wherein, if said heterocyclic group contains an —NH— moiety that nitrogen may be optionally substituted by a group selected from J; V is halo, nitro, cyano, hydroxy, oxo, trifluoromethyl, trifluoromethoxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂amino, C₁₋₆alkanoylamino, N—(C₁₋₆alkyl)carbamoyl, N,N—(C₁₋₆alkyl)₂carbamoyl, C₁₋₆alkylS(O)ₐ wherein a is 0 to 2, C₁₋₆alkoxycarbonyl, N—(C₁₋₆alkyl)sulphamoyl, N,N—(C₁₋₆alkyl)₂sulphamoyl or a group (D′-E′-); wherein V, including group (D′-E′-), may be optionally substituted on carbon by one or more W; W and Z are independently selected from halo, nitro, cyano, hydroxy, oxo, trifluoromethyl, trifluoromethoxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂amino, C₁₋₆alkanoylamino, N—(C₁₋₆alkyl)carbamoyl, N,N—(C₁₋₆alkyl)₂carbamoyl, C₁₋₆alkylS(O)ₐ wherein a is 0 to 2, C₁₋₆alkoxycarbonyl, N—(C₁₋₆alkyl)sulphamoyl or N,N—(C₁₋₆alkyl)₂sulphamoyl; G. J and K are independently selected from C₁₋₈alkyl, C₂₋₈alkenyl, C₂₋₈alkynyl, C₁₋₈alkanoyl, C₁₋₆alkylsulphonyl, C₁₋₈alkoxycarbonyl, carbamoyl, N—(C₁₋₈alkyl)carbamoyl, N,N—(C₁₋₈alkyl)carbamoyl, benzyloxycarbonyl, benzoyl and phenylsulphonyl, aryl, arylC₁₋₆alkyl or (heterocyclic group)C₁₋₆alkyl; wherein G, J and K may be optionally substituted on carbon by one or more Q; and wherein if said heterocyclic group contains an —NH— moiety that nitrogen may be optionally substituted by a group selected from hydrogen or C₁₋₆alkyl; Q is halo, nitro, cyano, hydroxy, oxo, trifluoromethyl, trifluoromethoxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂amino, C₁₋₆alkanoylamino, N—(C₁₋₆alkyl)carbamoyl, N,N—(C₁₋₆alkyl)₂carbamoyl, C₁₋₆alkylS(O)ₐ wherein a is 0 to 2, C₁₋₆alkoxycarbonyl, C₁₋₆alkoxycarbonylamino, N—(C₁₋₆alkyl)sulphamoyl, N,N—(C₁₋₆alkyl)₂sulphamoyl, aryl, aryloxy, arylC₁₋₆alkyl, arylC₁₋₆alkoxy, heterocyclic group, (heterocyclic group)C₁₋₆alkyl, (heterocyclic group)C₁₋₆alkoxy, or a group (D″-E″-); wherein Q. including group (D″-E″-), may be optionally substituted on carbon by one or more Z; D, D″ and D″ are independently selected from C₁₋₆alkyl, C₂₋₆alkenyl, C₂₋₆alkynyl, C₃₋₈cycloalkyl, C₃₋₈cycloalkylC₁₋₆alkyl, aryl, arylC₁₋₆alkyl, heterocyclic group, (heterocyclic group)C₁₋₆alkyl; wherein D, D′ and D″ may be optionally substituted on carbon by one or more F′; and wherein if said heterocyclic group contains an —NH— moiety that nitrogen may be optionally substituted by a group selected from K; E, E′ and E″ are independently selected from —N(R^{a})—, —O—, —C(O)O—, —OC(O)—, —C(O)—, —N(R^{a})C(O)—, —N(R^{a})C(O)N(R^{b})—, —N(R^{a})C(O)O—, —OC(O)N(R^{a})—, —C(O)N(R^{a})—, —S(O)ᵣ, —SO₂N(R^{a})—, —N(R^{a})SO₂—; wherein R^{a} and R^{b} are independently selected from hydrogen or C₁₋₆alkyl optionally substituted by one or more F and r is 0-2; F and F′ are independently selected from halo, nitro, cyano, hydroxy, trifluoromethyl, trifluoromethoxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂amino, C₁₋₆alkanoylamino, N—(C₁₋₆alkyl)carbamoyl, N,N—(C₁₋₆alkyl)₂carbamoyl, C₁₋₆alkylS(O)ₐ wherein a is 0 to 2, C₁₋₆alkoxycarbonyl, N—(C₁₋₆alkyl)sulphamoyl and N,N—(C₁₋₆alkyl)₂sulphamoyl; m is 0, 1, 2, 3 or 4; wherein the values of R¹ may be the same or different; Ring B is a ring selected from

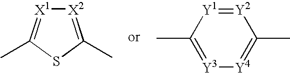

wherein, X¹ and X² are selected from CH or N. and Y¹, Y², Y³ and Y⁴ are selected from CH or N provided that at least one of Y¹, Y², Y³ and Y⁴ is N; R² is halo; n is 0, 1 or 2; wherein the values of R² may be the same or different; R³ is amino or hydroxy; R⁴ is halo, nitro, cyano, hydroxy, trifluoromethyl, trifluoromethoxy, amino, carboxy, carbamoyl, mercapto, sulphamoyl, C₁₋₃alkyl, C₂₋₃alkenyl, C₂₋₃alkynyl, C₁₋₃alkoxy, C₁₋₃alkanoyl, C₁₋₃alkanoyloxy, N—(C₁₋₃alkyl)amino, N,N—(C₁₋₃alkyl)₂amino, C₁₋₃alkanoylamino, N—(C₁₋₃alkyl)carbamoyl, N,N—(C₁₋₃alkyl)₂carbamoyl, C₁₋₃alkylS(O)ₐ wherein a is 0 to 2, C₁₋₃alkoxycarbonyl, N—(C₁₋₃alkyl)sulphamoyl, N,N—(C₁₋₃alkyl)₂sulphamoyl; and p is 0, 1 or 2; wherein the values of R⁴ may be the same or different; or a pharmaceutically acceptable salt or in vivo hydrolysable ester or amide thereof. See for example, compounds disclosed in WO2003092686.

In one aspect, the inhibitor of histone deacetylase activity may be an alpha-ketoepoxide compound, such as, for example, compounds disclosed in WO2003099272, having the general formula:

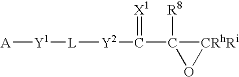

Wherein A is a cyclic moiety selected from the group consisting of C₃₋₁₄cycloalkyl, 3-14 membered heterocycloalkyl, C₄₋₁₄cycloalkenyl, 3-8 membered heterocycloalkenyl, aryl, and heteroaryl; the cyclic moiety being optionally substituted with alkyl, alkenyl, alkynyl, alkoxy, hydroxyl, hydroxylalkyl, halo, haloalkyl, amino, thio, alkylthio, arylthio, aralkylthio, acylthio, alkylcarbonyloxy, alkyloxycarbonyl, alkylcarbonyl, alkylsulfonylamino, aminosulfonyl, or alkylsulfonyl; or A is a saturated branched C₃₋₁₂hydrocarbon chain or an unsaturated branched C₃₋₁₂hydrocarbon chain optionally interrupted by —O—, —S—, —N(R^{a})—, —C(O)—, —N(R^{a})—SO₂—, —SO₂—N(R^{a})—, —N(R^{a})—C(O)—O—, —O— C(O)—N(R⁸)—, —N(R^{a})—C(O)—N(R^{b})—, —O—C(O)—, —C(O)—O—, —O—SO₂—, —SO₂—O—, or —O—C(O)—O—, where each of R^{a} and R^{b}, independently, is hydrogen, alkyl, alkenyl, alkynyl, alkoxy, hydroxylalkyl, hydroxyl, or haloalkyl; each of the saturated and the unsaturated branched hydrocarbon chain being optionally substituted with alkyl, alkenyl, alkynyl, alkoxy, hydroxyl, hydroxylalkyl, halo, haloalkyl, amino, thio, alkylthio, arylthio, aralkylthio, acylthio, alkylcarbonyloxy, alkyloxycarbonyl, alkylcarbonyl, alkylsulfonylamino, aminosulfonyl, or alkylsulfonyl; each of Y¹ and Y² independently, is —CH₂—, —O—, —S—, —N(R^{C})—, —N(R^{C})—C(O)—O—, —N(R^{C})—C(O)—, —C(O)—N(R^{C})—, —O—C(O)—N(R^{C})—, —N(R^{C})—C(O)—N(R^{d})—, —C(O)—, —C(NR^{C})—, —O—C(O)—O—, or a bond; each of R^{c} and R^{d}, independently, being hydrogen, alkyl, alkenyl, alkynyl, alkoxy, hydroxylalkyl, hydroxyl, or haloalkyl; L is a straight C₄₋₁₂hydrocarbon chain optionally containing at least one double bond, at least one triple bond, or at least one double bond and one triple bond; the hydrocarbon chain being optionally substituted with C₁₋₄alkyl, C₂₋₄alkenyl, C₂₋₄alkynyl, C₁₋₄alkoxy, hydroxyl, halo, amino, thio, alkylthio, arylthio, aralkylthio, acylthio, nitro, cyano, C₃₋₅cycloalkyl, 3-5 membered heterocycloalkyl, monocyclic aryl, 5-6 membered heteroaryl, C₁₋₄alkylcarbonyloxy, C₁₋₄alkyloxycarbonyl, C₁₋₄alkylcarbonyl, or formyl; and further being optionally interrupted by —O—, —N(R^{e})—, —N(R^{e})—C(O)—O—, —O—C(O)—N(R^{e})—, —N(R^{e})—C(O)—N(R^{f})—, or —O—C(O)—O—; each of R^{e} and R^{f}, independently, being hydrogen, alkyl, alkenyl, alkynyl, alkoxy, hydroxylalkyl, hydroxyl, or haloalkyl; X¹ is 0 or S; and each of R^{g}, R^{h}, and Rⁱ, independently, is hydrogen or C₁₋₆alkyl; provided that when each of Y¹ and Y² independently, is a bond or CH₂, A is unsubstituted phenyl or heterocyclyl, and L is C₄₋₇, L has at least one double bond or at least one triple bond, and when each of Y¹ and Y² is a bond, A is unsubstituted phenyl, and L is C₄, L is not a diene; or a salt thereof.

In one aspect, the inhibitor of histone deacetylase activity may be a benzamide derivative, such as, for example, compounds disclosed in WO2003087057, having the general formula:

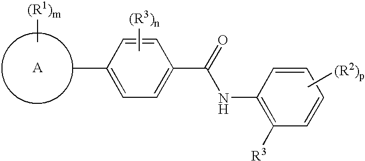

Wherein Ring A is a heterocyclyl, wherein if said heterocyclyl contains an —NH— moiety that nitrogen may be optionally substituted by a group selected from K; R¹ is a substituent on carbon and is selected from halo, nitro, cyano, hydroxy, trifluoromethyl, trifluoromethoxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C 6alkyl)₂amino, C₁₋₆alkanoylamino, N—(C₁₋₆alkyl)carbamoyl, N,N—(C₁₋₆alkyl)₂carbamoyl, C₁₋₆alkylS(O)ₐ wherein a is 0 to 2, C₁₋₆alkoxycarbonyl, N—(C₁₋₆alkyl)sulphamoyl, N,N—(C₁₋₆alkyl)₂sulphamoyl, aryl, aryloxy, arylC₁₋₆alkyl, heterocyclic group, (heterocyclic group)C₁₋₆alkyl, or a group (B-E-); wherein R¹, including group (B-E-), may be optionally substituted on carbon by one or more W; and wherein if said heterocyclic group contains an —NH— moiety that nitrogen may be optionally substituted by J; W is halo, nitro, cyano, hydroxy, trifluoromethyl, trifluoromethoxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂amino, C₁₋₆alkanoylamino, N—(C₁₋₆alkyl)carbamoyl, N,N—(C₁₋₆alkyl)₂carbamoyl, C₁₋₆alkylS(O)ₐ wherein a is 0 to 2, C₁₋₆alkoxycarbonyl, N—(C₁₋₆alkyl)sulphamoyl, N,N—(C₁₋₆alkyl)₂sulphamoyl, or a group (B′-E′-); wherein W including group (B′-E′-), may be optionally substituted on carbon by one or more Y; Y and Z are independently selected from halo, nitro, cyano, hydroxy, trifluoromethyl, trifluoromethoxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂amino; C₁₋₆alkanoylamino, N—(C₁₋₆alkyl)carbamoyl, N,N—(C₁₋₆alkyl)₂carbamoyl, C₁₋₆alkylS(o)ₐ wherein a is 0 to 2, C₁₋₆alkoxycarbonyl, N—(C₁₋₆alkyl)sulphamoyl or N,N—(C₁₋₆alkyl)₂sulphamoyl; G. J and K are independently selected from C₁₋₈alkyl, C₂₋₈alkenyl, C₁₋₈alkanoyl, C₁₋₈alkylsulphonyl, C₁₋₈alkoxycarbonyl, carbamoyl, N—(C₁₋₈alkyl)carbamoyl, N,N—(C₁₋₈alkyl)carbamoyl, benzyloxycarbonyl, benzoyl, phenylsulphonyl, aryl, arylC₁₋₆alkyl or (heterocyclic group)C₁₋₆alkyl; wherein G. J and K may be optionally substituted on carbon by one or more Q; and wherein if said heterocyclic group contains an —NH— moiety that nitrogen may be optionally substituted by hydrogen or C₁₋₆alkyl; Q is halo, nitro, cyano, hydroxy, trifluoromethyl, trifluoromethoxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂amino, C₁₋₆alkanoylamino, N—(C₁₋₆alkyl)carbamoyl, N,N—(C₁₋₆alkyl)₂carbamoyl, C₁₋₆alkylSO)ₐ wherein a is 0 to 2, C₁₋₆alkoxycarbonyl, C₁₋₆alkoxycarbonylamino, N—(C₁₋₆alkyl)sulphamoyl, N,N—(C₁₋₆alkyl)₂sulphamoyl, aryl, aryloxy, arylC₁₋₆alkyl, arylC₁₋₆alkoxy, heterocyclic group, (heterocyclic group)C₁₋₆alkyl, (heterocyclic group)C₁₋₆alkoxy, or a group (B″-E″-); wherein Q. including group (B″-E″-), may be optionally substituted on carbon by one or more Z; B, B′ and B″ are independently selected from C₁₋₆alkyl, C₂₋₆alkenyl, C₂₋₆alkynyl, C₃₋₈cycloalkyl, C₃₋₈cycloalkylC₁₋₆alkyl, aryl, arylC₁₋₆alkyl, heterocyclic group, (heterocyclic group)C₁₋₆alkyl, phenyl or phenylC₁₋₆alkyl; wherein B, B′ and B″ may be optionally substituted on carbon by one or more D; and wherein if said heterocyclic group contains an —NH— moiety that nitrogen may be optionally substituted by a group selected from G; E, E′ and E″ are independently selected from —N(R^{a})—, —O—, —C(O)O—, —OC(O)—, —C(O)—, —N(R^{a})C(O)—, —N(R^{a})C(O)N(R^{b})—, —N(R^{a})C(O)O—, —OC(O)N(R^{a})—, —C(O)N(R^{a})—, —S(⁰)ᵣ-, —SO₂N(R^{a})—, —N(R^{a})SO₂—; wherein R^{a} and R^{b} are independently selected from hydrogen or C₁₋₆alkyl optionally substituted by one or more F and r is 0-2; D and F are independently selected from halo, nitro, cyano, hydroxy, trifluoromethyl, trifluoromethoxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂amino, C₁₋₆alkanoylamino, N—(C₁₋₆alkyl)carbamoyl, N,N—(C₁₋₆alkyl)₂carbamoyl, C₁₋₆alkylS(O)ₐ wherein a is 0 to 2, C₁₋₆alkoxycarbonyl, N—(C₁₋₆alkyl)sulphamoyl or N,N—(C₁₋₆alkyl)₂sulphamoyl; m is 0, 1, 2, 3 or 4; wherein the values of R¹ may be the same or different; R is halo; n is 0, 1 or 2; wherein the values of R² may be the same or different; R³ is amino or hydroxy; R⁴ is halo, nitro, cyano, hydroxy, trifluoromethyl, trifluoromethoxy, amino, carboxy, carbamoyl, mercapto, sulphamoyl, C₁₋₃alkyl, C₂₋₃alkenyl, C₂₋₃alkynyl, C₁₋₃alkoxy, C₁₋₃alkanoyl, C₁₋₃alkanoyloxy, N—(C₁₋₃alkyl)amino, N,N—(C₁₋₃alkyl)₂amino, C₁₋₃alkanoylamino, N—(C₁₋₃alkyl)carbamoyl, N,N—(C₁₋₃alkyl)₂carbamoyl, C₁₋₃alkylS(O)ₐ wherein a is 0 to 2, C₁₋₃alkoxycarbonyl, N—(C₁₋₃alkyl)sulphamoyl, N,N—(C₁₋₃alkyl)₂sulphamoyl; p is 0, 1 or 2; wherein the values of R⁴ may be the same or different; or a pharmaceutically acceptable salt or in vivo hydrolysable ester or amide thereof; with the proviso that said compound is not N-(2-amino-6-hydroxyphenyl)-4-1-methylhomopiperazin-4-yl)benzamide; N-(2-amino-6-methylphenyl)-4-(1-methylhomopiperazin-4-yl)benzamide; N-(2-aminophenyl)-4-(1-t-butoxycarbonylhomopiperazin-4-yl)benzamide; or N-(2-aminophenyl)-4-(1-methylhomopiperazin-4-yl)benzamide.

In one aspect, the inhibitor of histone deacetylase activity may be a hydroxamic acid derivative, such as, for example, compounds disclosed in WO2003087066, having the general formula:

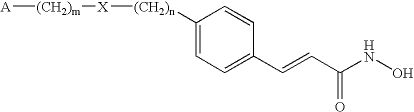

Wherein A is an optionally substituted phenyl or aromatic heterocyclic group which has 1 to 4 substituents selected from the group consisting of a halogen atom, a hydroxyl group, an amino group, a nitro group, a cyano group, an alkyl group having 1 to 4 carbons, an alkoxy group having 1 to 4 carbons, an aminoalkyl group having 1 to 4 carbons, an alkylamino group having 1 to 4 carbons, an acyl group lo having 1 to 4 carbons, an acylamino group having 1 to 4 carbons, an alkylthio group having 1 to 4 carbons, a perfluoroalkyl group having 1 to 4 carbons, a perfluoroalkoxy group having 1 to 4 carbons, a carboxyl group, an alkoxycarbonyl group having 1 to 4 carbons, a phenyl group, an aromatic heterocyclic group and a heterocyclic group, said heterocyclic group being optionally substituted with an 15 alkyl group having 1 to 4 carbons, a benzyl group, or a pyridylmethyl group; m is an integer of 0 to 4; n is an integer of 1 to 4; X is a moiety having a structure selected from those illustrated in formula

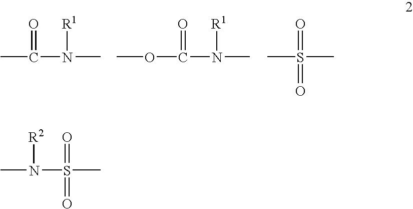

R¹ and R² are independently H or an optionally substituted alkyl group having 1 to 4 carbons; or a pharmaceutically acceptable salt thereof.

In one aspect, the inhibitor of histone deacetylase activity may be a sulfonyl derivative, such as, for example, compounds disclosed in WO2003076422, having the general formula:

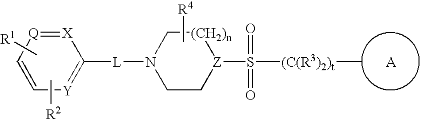

the N-oxide forms, the pharmaceutically acceptable addition salts and the stereo-chemically isomeric forms thereof, wherein 10 n is O. 1, 2 or 3 and when n is 0 then a direct bond is intended; t is O. 1, 2, 3 or 4 and when t is O then a direct bond is intended; each Q is nitrogen or; _each X is nitrogen or; _each Y is nitrogen or; —CH— 20 each Z is nitrogen or; Ri is —C(O)NR7R8, —N(H)C(O)R9, —C(O)—C′6alkanediylSR9, —NR^{o}C(O)N(OH)R9, —NR^{o}C(O)C6alkanediyl S. R9, —NR^{o}C(0)C═N(OH)R9 or another Zn chelating group 2 wherein R7 and Rx are each independently selected from hydrogen, hydroxy, C 6alkyl, hydroxyC 6alkyl, aminoC 6alkyl or aminoaryl; R9 is independently selected hydrogen, C 6alkyl, C′ 6alkylcarbonyl, arylC 6alkyl, C 6alkylpyrazinyl, pyridinone, pyrrolidinone or methylimidazolyl; R^{o} is independently selected hydrogen or C′ 6alkyl; R2 is hydrogen, halo, hydroxy, amino, nitro, C′ 6alkyl, C′ 6alkyloxy, trifluoromethyl, di(C 6alkyl)amino, hydroxyamino or naphtalenylsulfonylpyrazinyl; -L- is a direct bond or a bivalent radical selected from C 6alkanediyl, amino, carbonyl 35 or aminocarbonyl; each R3 represents a hydrogen atom and one hydrogen atom can be replaced by aryl; R4 is hydrogen, hydroxy, amino, hydroxyC′ 6alkyl, C1 6alkyl, C1 6alkyloxy, arylC 6alkyl, aminocarbonyl, hydroxycarbonyl, aminoC 6alkyl, aminocarbonylC 6alkyl, hydroxycarbonylC 6alkyl, hydroxyaminocarbonyl, C 6alkyloxycarbonyl, C 6alkylaminoC 6alkyl or di(C′ 6alkyl)aminoC 6alkyl; -) is a radical selected from Ps)s iR5)s JR6)s)s ¢: O 10 (a-1) (a-2) (a-3) (a-4) 6)s fR6)s fR6)s H (R)s N: e NHl (a-S) (a-6) (a-7) (a-8) fR6)s JR6)s P6)s)s N (a-9) (a-10) (a-11) (a-12) H iR6) s fR)s H:R (a-13) (a-14) (a-15) (a-16) &t;&t;CH:R s _,: (a-17) (a-) N (a-19) N (a-20) (6 30 :N (a-27) (a:-28) 6)s (a-25) (a-26) FIR s it (a-32) H 0} (31) t 0 (a-4 NO 1 - (a-41) (a-42) -123 O jR6)s O JR6)S O R6)SfR6)s/ ,NH 1 /( N /3:: (a-45) (a-46) (a-47)(a-48) 1 jR6)s fR6)s fR6)s N-/1 (a-49) (a-50) (a-S 1) wherein each s is independently 0, 1, 2, 3, 4 or 5; each Rs and R6 are independently selected from hydrogen; halo; hydroxy; amino; nitro; trihaloC 6alkyl; trihaloC 6alkyloxy; C 6alkyl; C 6alkyl substituted with aryl and C3 0cycloalkyl; Ci 6alkyloxy; C 6alkyloxyC 6alkyloxy; Ci 6alkylcarbonyl; C 6alkyloxycarbonyl; C 6alkylsulfonyl; cyanoC 6alkyl; hydroxyC 6alkyl; hydroxyC 6alkyloxy; hydroxyC 6alkylamino; aminoC 6alkyloxy; lO di(C-6alkyl)aminocarbonyl; di(hydroxyC 6alkyl)amino; (aryl)(C-6alkyl)amino; di(C 6alkyl)aminoC 6alkyloxy; di(C 6alkyl)aminoC 6alkylamino; di(C 6alkyl)aminoC 6alkylaminoC 6alkyl; arylsulfonyl; arylsulfonylamino; aryloxy; aryloxyC 6alkyl; arylC2 6alkenediyl; di(C 6alkyl)amino; di(C 6alkyl)aminoC 6alkyl; di(C 6alkyl)amino(C 6alkyl)amino; di(C 6alkyl)amino(C 6alkyl)aminoC 6alkyl; di(C 6alkyl)aminoC 6alkyl(C 6alkyl)amino; di(C-6alkyl)aminoC 6alkyl(C-6alkyl)aminoC 6alkyl; aminosulfonylamino(C 6alkyl)amino; aminosulfonylamino(C-6alkyl)aminoC 6alkyl; di(C 6alkyl)aminosulfonylamino(C 6alkyl)amino; di(C 6alkyl)aminosulfonylamino(C 6alkyl)aminoC 6alkyl; cyano; thiophenyl; thiophenyl substituted with di(C 6alkyl)aminoC 6alkyl(C 6alkyl)aminoC 6alkyl, di(C 6alkyl)aminoC 6alkyl, C 6alkylpiperazinylC 6alkyl, hydroxyC 6alkylpiperazinylC 6alkyl, hydroxyC 6alkyloxyC 6alkylpiperazinylC 6alkyl, di(C 6alkyl)aminosulfonylpiperazinylC 6alkyl, C 6alkyloxypiperidinyl, C 6alkyloxypiperidinylC 6alkyl, morpholinylC 6alkyl, hydroxyC 6alkyl(C-6alkyl)aminoC 6alkyl, or di(hydroxyC-6alkyl)aminoC 6alkyl; furanyl; furanyl substituted with hydroxyC 6alkyl; benzofuranyl; imidazolyl; oxazolyl; oxazolyl substituted with aryl and C 6alkyl; C 6alkyltriazolyl; tetrazolyl; pyrrolidinyl; pyrrolyl; piperidinylCI 6alkyloxy; morpholinyl; C1 6alkylmorpholinyl; morpholinylCI 6alkyloxy; morpholinylCI 6alkyl; morpholinylCI 6alkylamino; morpholinylC1 6alkylaminoCI 6alkyl; piperazinyl; C1 6alkylpiperazinyl; C1 6alkylpiperazinylCI 6alkyloxy; piperazinylCI 6alkyl; naphtalenylsulfonylpiperazinyl; naphtalenylsulfonylpiperidinyl; naphtalenylsulfonyl: CI 6alkylpiperazinylCI 6alkyl; C1 6alkylpiperazinylCI 6alkylamino; C 6alkylpiperazinylCI 6alkylaminoC1 6alkyl; C 6alkylpiperazinylsulfonyl; aminosulfonylpiperazinylC 6alkyloxy; aminosulfonylpiperazinyl; aminosulfonylpiperazinylCI 6alkyl; di(C 6alkyl)aminosulfonylpiperazinyl; di(CI 6alkyl)aminosulfonylpiperazinylC1 6alkyl; hydroxyC 6alkylpiperazinyl; hydroxyC 6alkylpiperazinylC 6alkyl; C1 6alkyloxypiperidinyl; C1 6alkyloxypiperidinylC 6alkyl; piperidinylaminoC 6alkylamino; piperidinylaminoC-6alkylaminoCI 6alkyl; 15 (CI 6alkylpiperidinyl)(hydroxyC′ 6alkyl)aminoC 6alkylamino; (C 6alkylpiperidinyl)(hydroxyC′ 6alkyl)aminoC 6alkylaminoCI 6alkyl; hydroxyC 6alkyloxyC 6alkylpiperazinyl; hydroxyC 6alkyloxyCI-6alkylpiperazinylC 6alkyl; (hydroxyC 6alkyl)(CI 6alkyl)amino; (hydroxyCI 6alkyl)(CI 6alkyl)aminoC 6alkyl; hydroxyC 6alkylaminoC 6alkyl; di(hydroxyCI 6alkyl)aminoC1 6alkyl; pyrrolidinylCI 6alkyl; pyrrolidinylCI 6alkyloxy; pyrazolyl; thiopyrazolyl; pyrazolyl substituted with two substituents selected from C 6alkyl or trihaloC 6alkyl; pyridinyl; pyridinyl substituted with C1 6alkyloxy′ aryloxy or aryl; pyrimidinyl; tetrahydropyrimidinylpiperazinyl; tetrahydropyrimidinylpiperazinylC 6alkyl; 2 quinolinyl; indolyl; phenyl; phenyl substituted with one, two or three substituents independently selected from halo, amino, nitro, C1 6alkyl, C 6alkyloxy, hydroxyC 4alkyl, trifluoromethyl, trifluoromethyloxy, hydroxyCI 4alkyloxy, C1 4alkylsulfonyl, C1 4alkyloxyCI 4alkyloxy, C1 4alkyloxycarbonyl, aminoC 4alkyloxy, di(C 4alkyl)aminoC 4alkyloxy, di(C 4alkyl)amino, di(CI 4alkyl)aminocarbonyl, di(C1 4alkyl)aminoC1 4alkyl, di(CI 4alkyl)aminoC1 4alkylaminoC1 4alkyl, di(CI 4alkyl)amino(C1 4alkyl)amino, di(C1 4alkyl)amino(C1 4alkyl)aminoCI 4alkyl, di(CI 4alkyl)aminoC1 4alkyl(CI 4alkyl)amino, di(CI 4alkyl)aminoC1 4alkyl(CI 4alkyl)aminoCI 4alkyl, 3 aminosulfonylamino(C′ 4alkyl)amino, aminosulfonylamino(C I 4alkyl)aminoCI 4alkyl, di(C 4alkyl)aminosulfonylamino(CI 4alkyl)amino, di(CI 4alkyl)aminosulfonylamino(C 4alkyl)aminoC′ 6alkyl, cyano, piperidinylC1 4alkyloxy, pyrrolidinylCI 4alkyloxy, aminosulfonylpiperazinyl, aminosulfonylpiperazinylC1 4alkyl, di(CI 4alkyl)aminosulfonylpiperazinyl, di(CI 4alkyl)aminosulfonylpiperazinylC1 4alkyl, hydroxyCI 4alkylpiperazinyl, hydroxyC′ 4alkylpiperazinylC1 4alkyl, C1 4alkyloxypiperidinyl, C1 4alkyloxypiperidinylC1 4alkyl, hydroxyC1 4alkyloxyC1 4alkylpiperazinyl, hydroxyCI 4alkyloxyCI 4alkylpiperazinylCI 4alkyl, (hydroxyCI 4alkyl)(C1 4alkyl)amino, (hydroxyC1 4alkyl)(CI 4alkyl)aminoCI 4alkyl, di(hydroxyC1 4alkyl)amino, di(hydroxyCI 4alkyl)aminoC1 4alkyl, furanyl, furanyl substituted with —CH═CH—CH═CH—, pyrrolidinylCI 4alkyl, pyrrolidinylC′ 4alkyloxy, morpholinyl, morpholinylCI 4alkyloxy, morpholinylCI 4alkyl, morpholinylCI 4alkylamino, morpholinylCI 4alkylaminoC1 4alkyl, piperazinyl, C1 4alkylpiperazinyl, C1 4alkylpiperazinylCI 4alkyloxy, piperazinylCI 4alkyl, C1 4alkylpiperazinylC1 4alkyl, C1 4alkylpiperazinylC1 4alkylamino, CI 4alkylpiperazinylCI 4alkylaminoCI 6alkyl, tetrahydropyrimidinylpiperazinyl, tetrahydropyrimidinylpiperazinylCI 4alkyl, piperidinylaminoCI 4alkylamino, piperidinylaminoC1 4alkylaminoCI 4alkyl, (CI 4alkylpiperidinyl)(hydroxyC 4alkyl)aminoCI 4alkylamino, (C1 4alkylpiperidinyl)(hydroxyCI 4alkyl)aminoC1 4alkylaminoC1 4alkyl, pyridinylCI 4alkyloxy, hydroxyCI 4alkylamino, hydroxyCI 4alkylaminoCI 4alkyl, di(CI 4alkyl)aminoC1 4alkylamino, aminothiadiazolyl, aminosulfonylpiperazinylCI 4alkyloxy, or thiophenylC 4alkylamino; (CH2)n the central/moiety may also be bridged (i.e. forming a bicyclic moiety) with a methylene, ethylene or propylene bridge; 2 each R5 and R6 can be placed on the nitrogen in replacement of the hydrogen; aryl in the above is phenyl, or phenyl substituted with one or more substituents each independently selected from halo, C′ 6alkyl, C 6alkyloxy, trifluoromethyl, cyano or hydroxycarbonyl.

In one aspect, the inhibitor of histone deacetylase activity may be a trihalomethylcarbonyl compound, such as, for example, compounds disclosed in WO2003099760, having the general formula:

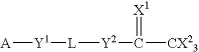

wherein A is a cyclic moiety selected from the group consisting of C34 j cycloalkyl, 3-14 membered heterocycloalkyl, C44 cycloalkenyl, 3-8 membered heterocycloalkenyl, aryl, and heteroaryl; the cyclic moiety being optionally substituted with alkyl, alkenyl, alkynyl, alkoxy, hydroxyl, hydroxylalkyl, halo, I haloalkyl, amino, thio, alkylthio, arylthio, aralkylthio, acylthio, alkylcarbonyloxy,] 10 aLkyloxycarbonyl, alkylcarbonyl, alkylsulfonylamino, aminosulfonyl, or alkylsulfonyl; or A is a saturated branched C3-2 hydrocarbon chain or an unsaturated branched C3-2 hydrocarbon chain optionally interrupted by —O—, —S—, —N(Ra)—, —C(O)—, —N(Ra)-SO2-, —SO2-N(Ra)-, —N(Ra)-C(0)-0-, -0-C(0)-N(Ra)-, —N(Ra)-C(0)-N(Rb)-, —O—C(O)—, —C(O)—O—, —O—SO2-, —SO2-O—, or —O—C(O)—O—, where each of Ra and Rb, characterized independently, is hydrogen, alkyl, alkenyl, alkynyl, alkoxy, hydroxylalkyl, hydroxyl, or haloalkyl; each of the saturated and the unsaturated branched hydrocarbon chain being optionally substituted with alkyl, alkenyl, alkynyl, alkoxy, hydroxyl, hydroxylalkyl, halo, haloalkyl, amino, thio, alkylthio, arylthio, aralkylthio, acylthio, alkylcarbonyloxy, alkyloxycarbonyl, alkylcarbonyl, alkylsulfonylamino, I aminosulfonyl, or aLkylsulfonyl; each of Y and y2, independently, is —O—, —S—, —N(RC)—, —N(RC)—C(0)-O—, —N(RC)—C(0)-, —C(0)-N(RC)—, —O—C(0)-N(RC)—, —N(RC)—C(0)-N(Rd)-, —O—C(0)-O—, or a bond; each of Rc and R0, independently, being hydrogen, alkyl, alkenyl, alkynyl, alkoxy, hydroxylalkyl, hydroxyl, or haloalkyl; 25 L is a straight C3-2 hydrocarbon chain optionally containing at least one double bond, at least one triple bond, or at least one double bond and one triple bond; the hydrocarbon chain being optionally substituted with Ci4 alkyl, C2 4 aLkenyl, C2 4alkynyl, Ci4 alkoxy, hydroxyl, halo, amino, thio, alkylthio, arylthio, aralkylthio, acylthio, nitro, cyano, C3s cycloalkyl, 3-5 membered heterocycloalkyl, monocyclic I aryl, 5-6 membered heteroaryl, C1 4 alkylcarbonyloxy, C1 4 alkyloxycarbonyl, C1 4 alkylcarbonyl, or formyl; and further being optionally interrupted by —O—, —N(Re)-, —N(Re)-C(0)-O—, —O—C(0)-N(Re)-, —N(Re)-C(0)-N(Rf)-, or —O—C(O)—O—; each of Re and Rf, independently, being hydrogen, aLkyl, alkenyl, aLkynyl, alkoxy, hydroxylalkyl, hydroxyl, or haloalkyl, and I X is O or S; X2 is a halogen; provided that when Ye and y2 are each a bond, L is a C6 2 hydrocarbon chain 10 containing at least one double bond at C1, C2, C3 or C5 of the hydrocarbon chain I from C═X, at least one triple bond, or at least one double bond and one triple bond, the hydrocarbon chain being optionally substituted with Ci4 alkyl, C2 4 alkenyl, C2 4 alkynyl, C4 alkoxy, hydroxyl, halo, amino, thio, alkylthio, arylthio, aralkylthio, acylthio, nitro, cyano, C35 cycloalkyl, 3-5 membered heterocycloalkyl, monocyclic characterized aryl, 5-6 membered heteroaryl, C4 alkylcarbonyloxy, Ci 4 alkyloxycarbonyl, Ci4 alkylcarbonyl, or formyl; and further being optionally interrupted by —O—, —N(Re)-, —N(Re)-C(0)-O—, —O—C(0)-N(Re)-, —N(Re)-C(0)-N(Rf)-, or —O—C(O)—O—; each of Re and IRf, independently, being hydrogen, alkyl, aLkenyl, alkynyl, alkoxy, hydroxylalkyl, hydroxyl, or haloalkyl; or a salt thereof.

In one aspect, the inhibitor of histone deacetylase activity may be an alpha-chalcogenmethylcarbonyl compound, such as, for example, compounds disclosed in WO2003099789, having the general formula:

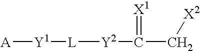

wherein A is a cyclic moiety selected from the group consisting of C34 cycloalkyl, 3-14 membered heterocycloalkyl, C44 cycloalkenyl, 3-8 membered heterocycloalkenyl, aryl, and heteroaryl; the cyclic moiety being optionally substituted with alkyl, alkenyl, alkynyl, alkoxy, hydroxyl, hydroxylalkyl, halo, haloalkyl, amino, thio, alkylthio, arylthio, aralkylthio, acylthio, alkylcarbonyloxy, 10 alkyloxycarbonyl, alkylcarbonyl, alkylsulfonylamino, aminosulfonyl, or alkylsulfonyl; or A is a saturated branched C3-2 hydrocarbon chain or an unsaturated i branched C3-2 hydrocarbon chain optionally interrupted by —O—, —S—, —N(Ra)-, —C(O)—, —N(Ra)-SO2-, —SO2-N(Ra)-′ —N(Ra)-C(0)-0-, -0-C(0)-N(Ra)-, —N(Ra)-C(0)-N(Rb)-, —O—C(O)—, —C(O)—O—, —O—SO2-, —SO2-O—, or —O—C(O)—O—, where each of Ra and Rb, characterized independently, is hydrogen, alkyl, alkenyl, alkynyl, alkoxy, hydroxylalkyl, hydroxyl, or haloalkyl; each of the saturated and the unsaturated branched hydrocarbon chain being optionally substituted with alkyl, alkenyl, alkynyl, alkoxy, hydroxyl, hydroxylalkyl, halo, haloalkyl, amino, thio, alkylthio, arylthio, aralkylthio, acylthio, alkylcarbonyloxy, alkyloxycarbonyl, alkylcarbonyl, alkylsulfonylamino, aminosulfonyl, or alkylsulfonyl; each of Y and y2, independently, is —CH2-, —O—, —S—, —N(RC)—, —N(RC)—C(0)-O—, —N(RC)—C(0)-, —C(0)-N(RC)—, —O—C(0)-N(RC)—, —N(RC)—C(0)-N(Rd)-, —O—C(O)—O—, or a bond; each of Rc and Rd. independently, being hydrogen, alkyl, alkenyl, alkynyl, alkoxy, hydroxylalkyl, hydroxyl, or haloalkyl; 25 L is a straight C3-2 hydrocarbon chain optionally containing at least one double bond, at least one triple bond, or at least one double bond and one triple bond; the hydrocarbon chain being optionally substituted with C′4 alkyl, C2 4 alkenyl, C2 4 alkynyl, C4 alkoxy, hydroxyl, halo, amino, thio, alkylthio, arylthio, aralkylthio, acylthio, nitro, cyano, C35 cycloalkyl, 3-5 membered heterocycloalkyl, monocyclic aryl, 5-6 membered heteroaryl, C′4 alkylcarbonyloxy, C4 alkyloxycarbonyl, C4 alkylcarbonyl, or formyl; and further being optionally interrupted by —O—, —N(Re)-, —N(Re)-C(0)-O—, —O—C(0)-N(Re)-, —N(Re)-C(0)-N(Rf)-, or —O—C(O)—O—; each of Re and 5 Rf, independently, being hydrogen, alkyl, alkenyl, alkynyl, alkoxy, hydroxylalkyl, hydroxyl, or haloalkyl; X is O or S; and X2 is —OR, —SR′, or —SeRi, wherein R is hydrogen, alkyl, acyl, aryl or aralkyl; 10 provided that when Yt is a bond and L is saturated, the carbon adjacent to Y is not substituted with C′4 alkoxy or hydroxyl; or a salt thereof.

In one aspect, the inhibitor of histone deacetylase activity may be bicyclic hydroxamate derivative, such as, for example, compounds disclosed in WO2003066579, having the general formula:

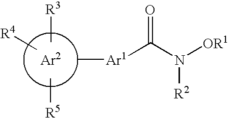

Wherein R′ is hydrogen or alkyl; R2 is hydrogen; Ar′ is phenylene or a six membered heteroarylene ring containing one or two nitrogen ring atoms, the rest of the ring atoms being carbon; wherein said Ar′ group is optionally substituted with one or two groups independently selected from alkyl, halo, hydroxy, alkoxy, haloalkoxy, or haloalkyl; 15 Ar2 is aryl, benzimidazol-2-yl, cycloalkyl or heterocycloalkyl; R3 is hydrogen, alkyl, halo, hydroxy, or alkoxy; and R4 and R5 are independently selected from the group consisting of hydrogen, alkyl, halo, haloalkyl, nitro, cyano, carboxy, carboxyalkyl, alkoxycarbonyl, optionally substituted phenyl, optionally substituted heteroaryl, optionally substituted heterocycloalkyl, cycloalkyl, 20 heterocycloaminoalkyl, —X—R6, or —(C _{—}6alkylene)-Y—R7 where X and Y are independently —O—, —S—, —SO— —SO2- —NRs-, —CO— —NR9Co- —CoNRo- —NRI1So2- —So2NRI2- —NHC(o)o- —OC(0)NH—, —NR 3CoNR′4-, or —NR 5SO2NR 6- where R6 and R7 are independently hydrogen, alkyl, hydroxyalkyl, optionally substituted phenyl, optionally substituted heteroaryl, optionally substituted heterocycloalkyl, cycloalkyl, optionally substituted phenylalkyl, optionally 25 substituted phenoxyalkyl, optionally substituted phenylalkenyl, optionally substituted phenylaminoalkyl, optionally substituted heteroaralkyl, optionally substituted heteroaryloxyalkyl, optionally substituted heterocycloalkylalkyl, or cycloalkylalkyl, R8, R9, R, Ri3, and R's are independently hydrogen, alkyl, hydroxyalkyl, alkoxyalkyl, or optionally substituted phenylalkyl; R′^{o}, R 2, R 4, and Ri6 are independently hydrogen, alkyl, optionally 30 substituted phenylalkyl, alkoxy, hydroxyalkyl, haloalkyl, alkoxyalkyl, carboxyalkyl, cyanoalkyl, aminoalkyl, aminocarbonylalkyl, alkylaminocarbonylalkyl, dialkylaminocarbonylalkyl, or acyl or R4 and Rs together form methylenedioxy; and individual isomers, mixtures of isomers; or a pharmaceutically acceptable salt thereof provided that: (i) at least one of R3, R4 and Rs is not hydrogen; (ii) when Ar2 is cycloalkyl, then at least two of R3, 35 R4 and Rs are hydrogen; (iii) when R′ and R3 are hydrogen, Ar′ is phenylene and Ar2 is phenyl, and one of R4 and R5 is methoxy, then the other of R4 and R5 is not _oR6 where R6 is S cyclopentyl or phenylpentyl; (iv) when Ar′ is phenylene and Ar2 is phenyl then at least one of R3, R4 and R5 is not alkyl; (v) when Ar′ is phenylene, Ar2 is aryl and is located at the 3 position of the phenylene ring, then Ar2 is not substituted with an optionally substituted phenyl; (vi) when Ar′ is phenylene and Ar2 is phenyl, and R4 or R5 is —CONRiOR6 or —(C, 6alkylene)-CONR′^{o}R7 then said R4 or R5 is not located at the 4-position of the phenyl ring; and 10 (vii) when Ar′ is phenylene and Ar2 is phenyl and two of R3, R4 and R5 are hydrogen, then the remaining of R3, R4 and R5 is not nitro.

In one aspect, the inhibitor of histone deacetylase activity may be a compound of the general formula:

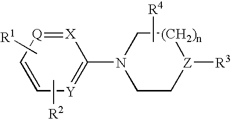

the N-oxide forms, the pharmaceutically acceptable addition salts and the stereo chemically isomeric forms thereof, wherein n is 0, 1, 2 or 3 and when n is 0 then a direct bond is intended; each Q is nitrogen or; each X is nitrogen or; each Y is nitrogen or; —CH— each Z is nitrogen or; R′ is —C(o)NR5R6, —N(H)C(O)R7, —C(O)—C′ 6alkanediylSR7, —NR8C(O)N(OH)R7, —NR8C(O)C 6alkanediylSR7, —NR8C(o)C═N(oH)R7 or another Zn-chelating-group wherein R5 and R6 are each independently selected from hydrogen, hydroxy, C′ 6alkyl, hydroxyC′ 6alkyl, aminoC 6alkyl or aminoaryl; R7 is independently selected from hydrogen, C′ 6alkyl, C 6alkylcarbonyl, arylC′ 6alkyl, C′ 6alkylpyrazinyl, pyridinone, pyrrolidinone or methylimidazolyl; R8 is independently selected from hydrogen or C′ 6alkyl; R2 is hydrogen, halo, hydroxy, amino, nitro, C′ 6alkyl, C′ 6alkyloxy, trifluoromethyl, di(C′ 6alkyl)amino, hydroxyamino or naphtalenylsulfonylpyrazinyl; R3 is hydrogen, C′ 6alkyl, arylC2 6alkenediyl, furanylcarbonyl, naphtalenylcarbonyl, —C(O)phenylR9, C 6alkylaminocarbonyl, aminosulfonyl, arylaminosulfonyl, aminosulfonylamino, di(C 6alkyl)aminosulfonylamino, arylaminosulfonylamino, aminosulfonylaminoC 6alkyl, di(C′ 6alkyl)aminosulfonylaminoC′ 6alkyl, arylaminosulfonylaminoC 6alkyl, di(C 6alkyl)aminoC 6alkyl, C″2alkylsulfonyl, di(C′ 6alkyl)aminosulfonyl, trihaloC′ 6alkylsulfonyl, di(aryl)CI 6alkylcarbonyl, thiophenylCI 6alkylcarbonyl, pyridinylcarbonyl or arylC 6alkylcarbonyl wherein each R9 is independently selected from phenyl; phenyl substituted with one, two or three substituents independently selected from halo, amino, C 6alkyl, 5 C 6alkyloxy, hydroxyC 4alkyl, hydroxyC 4alkyloxy, aminoC 4alkyloxy, di(C 4alkyl)aminoC 4alkyloxy, di(C 6alkyl)aminoC 6alkyl, di(C 6alkyl)aminoC 6alkyl(C 6alkyl)aminoC 6alkyl, hydroxyC 4alkylpiperazinylC 4alkyl, C 4alkyloxypiperidinylC 4alkyl, hydroxyC 4alkyloxyC 4alkylpiperazinyl, C 4alkylpiperazinylC 4alkyl, di(hydroxyC 4alkyl)aminoC 4alkyl, pyrrolidinylC 4alkyloxy, morpholinylC 4alkyloxy, or morpholinylC 4alkyl; thiophenyl; or thiophenyl substituted with di(C 4alkyl)aminoC 4alkyloxy, di(C 6alkyl)aminoC 6alkyl, di(C 6alkyl)aminoC 6alkyl(C 6alkyl)aminoC 6alkyl, pyrrolidinylC 4alkyloxy, C 4alkylpiperazinylC 4alkyl, di(hydroxyC 4alkyl)aminoC 4alkyl or morpholinylC 4alkyloxy. R4 is hydrogen, hydroxy, amino, hydroxyC 6alkyl, C 6alkyl, C 6alkyloxy, arylC 6alkyl, aminocarbonyl, hydroxycarbonyl, aminoC 6alkyl, aminocarbonylC 6alkyl, hydroxycarbonylC 6alkyl, hydroxyaminocarbonyl, C 6alkyloxycarbonyl, C 6alkylaminoC 6alkyl or di(C 6alkyl)aminoC 6alkyl; when R3 and R4 are present on the same carbon atom, R3 and R4 together may form a bivalent radical of formula I —C(0)-NH—CH2-NRI^{o}- (a-1) wherein R1^{o} is hydrogen or aryl; when R3 and R4 are present on adjacent carbon atoms, R3 and R4 together may form a bivalent radical of formula ═CH—CH═CH—CH═ (b-1); aryl in the above is phenyl, or phenyl substituted with one or more substituents each independently selected from halo, C′ 6alkyl, C, 6alkyloxy, trifluoromethyl, cyano or hydroxycarbonyl. See for example, compounds disclosed in WO2003075929.

In one aspect, the inhibitor of histone deacetylase activity may be a carbonylamino derivative, such as, for example, compounds disclosed in WO2003076395, having the general formula:

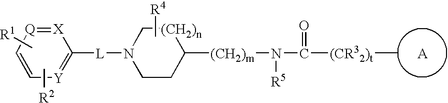

the N-oxide forms, the pharmaceutically acceptable addition salts and the stereo chemically isomeric forms thereof, wherein n is 0, 1, 2 or 3 and when n is 0 then a direct bond is intended; m is O or 1 and when m is 0 then a direct bond is intended; t is 0, 1, 2, 3 or 4 and when t is 0 then a direct bond is intended; each Q is nitrogen or; each X is nitrogen or; each Y is nitrogen or; R1 is —C(0)NR8R9, —NHC(0)R1^{o}, —C(O)—C1 6alkanediylSR1^{o}, —NR11C(0)N(OH)R1^{o}, —NR11C(O)C1 6alkanediylSR1^{o}, —NR11C(0)C═N(OH)R1^{o} or another Zn-chelating group wherein R8 and R9 are each independently selected from hydrogen, hydroxy, C1 6alkyl, hydroxyC1 6alkyl, aminoC1 6alkyl or aminoaryl; R1^{o} is hydrogen, C1 6alkyl, C1 6alkylcarbonyl, arylC1 6alkyl, C1 6alkylpyrazinyl, pyridinone, pyrrolidinone or methylimidazolyl; R11 is hydrogen or C1 6alkyl; R2 is hydrogen, halo, hydroxy, amino, nitro, Cat 6alkyl, C1 6alkyloxy, trifluoromethyl, di(C1 6alkyl)amino, hydroxyamino or naphtalenylsulfonylpyrazinyl; -L- is a direct bond or a bivalent radical selected from Cat 6alkanediyl, Cat 6alkanediyloxy, amino, carbonyl or aminocarbonyl; each R3 independently represents a hydrogen atom and one hydrogen atom can be replaced by a substituent selected from aryl; R4 is hydrogen, hydroxy, amino, hydroxyC1 6alkyl, C1 6alkyl, C1 6alkyloxy, arylC 6alkyl, aminocarbonyl, hydroxycarbonyl, aminoC 6alkyl, aminocarbonylC 6alkyl, hydroxycarbonylC 6alkyl, hydroxyaminocarbonyl, C 6alkyloxycarbonyl, C 6alkylaminoC 6alkyl or di(C 6alkyl)aminoC 6alkyl; R5 is hydrogen, C 6alkyl, C3 0cycloalkyl, hydroxyC 6alkyl, C 6alkyloxyC 6alkyl, 1 di(C 6alkyl)aminoC 6alkyl or aryl; -) is a radical selected from P6)s 6)s jR7) s)s [: ¢,: N (a-1) (a-2) (a-3) (a-4) 7)s iR7)s 7)s H)s N 1 N 1 (a-S) (a-6) (a-7) (a-8) 7)s fR7)s 7)s 7)s N (a-9) (a-10) (a-11) (a-12)-jR)g R)s H/R7 20 (a-13) (a-14) (a-15) (a-16) 1-53 3 )s 6 6)s N=/ N/: ′Cog b (a-17) (a-18) IN (a-19) N(a-20) 6)s 7) s 7)s 7)s ON′ (a-21) (a-22) (a-23) (a-24) s 7)s 7)s 6)s H)s O H (a-25) (a-26) (a-27) (a-28) 7)s 7)s 7)s 7)s H=IN ¢&t;O&t; (a-29) (a-30) (a-31) (a-32) (R)s 1 (R7)O (R7) Is AN NO I N: &t; N (a-33) (a-34) (a-35) (a-36) /(R7) /(R7)s 7)s 7)s NO iO I (a-37) (a-3X) (a-39) (a-40) -54 jR7)s iR7)s fR6)s fR7)s f/ N-/ N,J (a-41) (a-42) (a-43) (a-44) O JR7)S O JR7)S O IR7)S fR7)s Ng /NH /, Ng /: (a-45) (a-46) (a-47) (a-48) 1 JR7)S fR7)s JR7)s 53 N-/) (a-49) (a-50) (a-51) wherein each s is independently 0, 1, 2, 3, 4 or 5; each R6 and R7 are independently selected from hydrogen; halo; hydroxy; amino; nitro; trihaloC 6alkyl; trihaloC 6alkyloxy; C 6alkyl; C 6alkyl substituted with aryl and C3 0cycloalkyl; C 6alkyloxy; C 6alkyloxyC 6alkyloxy; Ct 6alkylcarbonyl; 1 C 6alkyloxycarbonyl; C 6alkylsulfonyl; cyanoC 6alkyl; hydroxyC 6alkyl; hydroxyC 6alkyloxy; hydroxyC 6alkylamino; aminoC1 6alkyloxy; di(C1L 6alkyl)aminocarbonyl; di(hydroxyC 6alkyl)amino; (aryl)(C 6alkyl)amino; di(Ct 6alkyl)aminoC 6alkyloxy; di(C 6alkyl)aminoC 6alkylamino; di(C 6alkyl)aminoC 6alkylaminoC 6alkyl; arylsulfonyl; arylsulfonylamino; aryloxy; aryloxyC 6alkyl; arylC2 6alkenediyl; di(C 6alkyl)amino; di(C 6alkyl)aminoC 6alkyl; di(C1 6alkyl)amino(C 6alkyl)amino; di(C 6alkyl)amino(C 6alkyl)aminoC 6alkyl; di(C 6alkyl)aminoC 6alkyl(C 6alkyl)amino; di(C 6alkyl)aminoC 6alkyl(C 6alkyl)aminoC 6alkyl; 2 aminosulfonylamino(C 6alkyl)amino; aminosulfonylamino(C-6alkyl)aminoC 6alkyl; di(Ci 6alkyl)aminosulfonylamino(C 6alkyl)amino; di(C 6alkyl)aminosulfonylamino(C 6alkyl)aminoC 6alkyl; cyano; thiophenyl; thiophenyl substituted with di(C 6alkyl)aminoC 6alkyl(C 6alkyl)aminoC 6alkyl, 2 di(C 6alkyl)aminoC 6alkyl, C 6alkylpiperazinylC 6alkyl, hydroxyC 6alkylpiperazinylC 6alkyl, hydroxyC 6alkyloxyC 6alkylpiperazinylC 6alkyl, di(C1 6alkyl)aminosulfonylpiperazinylC1 6alkyl, C1 6alkyloxypiperidinyl, C1 6alkyloxypiperidinylC1 6alkyl, morpholinylC1 6alkyl, hydroxyC1 6alkyl(C1 6alkyl)aminoC1 6alkyl, or di(hydroxyc1 6alkyl)aminoC1 6alkyl; furanyl; furanyl substituted with hydroxyC1 6alkyl; benzofuranyl; imidazolyl; oxazolyl; oxazolyl substituted with aryl and C1 6alkyl; C1-6alkyltriazolyl; tetrazolyl; pyrrolidinyl; pyrrolyl; piperidinylC1 6alkyloxy; morpholinyl; C1 6alkylmorpholinyl; morpholinylC1 6alkyloxy; morpholinylC1 6alkyl; morpholinylC1 6alkylamino; morpholinylC1 6alkylaminoC 6alkyl; piperazinyl; C1 6alkylpiperazinyl; 10 C1-6alkylpiperazinyl C1-6alkyloxy; piperazinyl C1-6alkyl; naphtalenylsulfonylpiperazinyl; naphtalenylsulfonylpiperidinyl; naphtalenylsulfonyl; C1 6alkylpiperazinylC1 6alkyl; C1 6alkylpiperazinylC 6alkylamino; C1 6alkylpiperazinylC1 6alkylaminoC1 6alkyl; C1 6alkylpiperazinylsulfonyl; aminosulfonylpiperazinylC1 6alkyloxy; aminosulfonylpiperazinyl; aminosulfonylpiperazinylC1 6alkyl; di(C1 6alkyl)aminosulfonylpiperazinyl; di(C1 6alkyl)aminosulfonylpiperazinylC1 6alkyl; hydroxyC1 6alkylpiperazinyl; hydroxyC1 6alkylpiperazinylC1 6alkyl; C1 6alkyloxypiperidinyl; C1 6alkyloxypiperidinylC 6alkyl; piperidinylaminoC1 6alkylamino; piperidinylaminoC1 6alkylaminoC1 6alkyl; (C1 6alkylpiperidinyl)(hydroxyC 6alkyl)aminoC1 6alkylamino, (C1 6alkylpiperidinyl)(hydroxyC 6alkyl)aminoC1 6alkylaminoC1 6alkyl; hydroxyC 6alkyloxyC1-6alkylpiperazinyl; hydroxyC 6alkyloxyC1-6alkylpiperazinylC1 6alkyl; (hydroxyC1 6alkyl)(C1 6alkyl)amino; (hydroxyC1 6alkyl)(C1 6alkyl)aminoC1 6alkyl; hydroxyC1 6alkylaminoC1 6alkyl; di(hydroxyC 6alkyl)aminoC1 6alkyl; pyrrolidinylCI 6alkyl; pyrrolidinylC1 6alkyloxy; pyrazolyl; thiopyrazolyl; pyrazolyl substituted with two substituents selected from C 6alkyl or trihaloC1 6alkyl; pyridinyl; pyridinyl substituted with C1 6alkyloxy, aryloxy or aryl; pyrimidinyl; tetrahydropyrimidinylpiperazinyl; tetrahydropyrimidinylpiperazinylC1 6alkyl; quinolinyl; indole; phenyl; phenyl substituted with one, two or three substituents independently selected from halo, amino, nitro, C1 6alkyl, C1 6alkyloxy, hydroxyC1 4alkyl, trifluoromethyl, trifluoromethyloxy, hydroxyC1 4alkyloxy, C1 4alkylsulfonyl, C1 4alkyloxyC1 4alkyloxy, C1 4alkyloxycarbonyl, aminoC1 4alkyloxy, di(C1 4alkyl)aminoC1 4alkyloxy, di(C1 4alkyl)amino, di(C1 4alkyl)aminocarbonyl, di(C1 4alkyl)aminoC1 4alkyl, di(C1 4alkyl)aminoC 4alkylaminoC1 4alkyl, di(C1 4alkyl)amino(c1 4alkyl)amino, di(c1-4alkyl)amino(c1-4alkyl)aminoc-4alkyl′ di(C1 4alkyl)aminoC1 4alkyl(C1 4alkyl)amino, di(C1 4alkyl)aminoC1 4alkyl(C1 4alkyl)aminoC1 4alkyl, aminosulfonylamino(C1 4alkyl)amino, aminosulfonylamino(C1 4alkyl)aminoC1 4alkyl, di(C1 4alkyl)aminosulfonylamino(C1 4alkyl)amino, S di(C1 4alkyl)aminosulfonylamino(C1 4alkyl)aminoC1 6alkyl, cyano, piperidinylC1 4alkyloxy, pyrrolidinylC1 4alkyloxy, aminosulfonylpiperazinyl, aminosulfonylpiperazinylC1 4alkyl, di(C1 4alkyl)aminosulfonylpiperazinyl, di(C1 4alkyl)aminosulfonylpiperazinylC1 4alkyl, hydroxyC1 4alkylpiperazinyl, hydroxyC1 4alkylpiperazinylC1 4alkyl, C1 4alkyloxypiperidinyl, C1 4alkyloxypiperidinylC1 4alkyl, hydroxyC1 4alkyloxyC1 4alkylpiperazinyl, hydroxyC1 4alkyloxyC1 4alkylpiperazinylC1 4alkyl, (hydroxyC1 4alkyl)(C1 4alkyl)amino, (hydroxyC1 4alkyl)(C1 4alkyl)aminoC1 4alkyl, di(hydroxyC1 4alkyl)amino, di(hydroxyC1 4alkyl)aminoC1 4alkyl, furanyl, furanyl substituted with —CH═CH—CH═CH—, pyrrolidinylC1 4alkyl, pyrrolidinylC1 4alkyloxy, 1S morpholinyl, morpholinylC1 4alkyloxy, morpholinylC1 4alkyl, morpholinylC1 4alkylamino, morpholinylC1 4alkylaminoC1 4alkyl, piperazinyl, C1 4alkylpiperazinyl, C1 4alkylpiperazinylC1 4alkyloxy, piperazinylC1 4alkyl, C1 4alkylpiperazinylC1 4alkyl, C1 4alkylpiperazinylC1 4alkylamino, C1 4alkylpiperazinylC1 4alkylaminoC1 6alkyl, tetrahyfropyrimidinylpiperazinyl, tetrahydropyrimidinylpiperazinylC1 4alkyl, piperidinylaminoC1 4alkylamino, piperidinylaminoC1 4alkylaminoC1 4alkyl, (C1 4alkylpiperidinyl)(hydroxyCI 4alkyl)aminoC1 4alkylamino, (C1 4alkylpiperidinyl)(hydroxyC 4alkyl)aminoC1 4alkylaminoC1 4alkyl, pyridinylC1 4alkyloxy, hydroxyC1 4alkylamino, hydroxyC1 4alkylaminoC1 4alkyl, di(C1 4alkyl)aminoC1 4alkylamino, aminothiadiazolyl, aminosulfonylpiperazinylC1 4alkyloxy, or thiophenylC_{1 4}alkylamino; each R6 and R7 can be placed on the nitrogen in replacement of the hydrogen; aryl in the above is phenyl, or phenyl substituted with one or more substituents each independently selected from halo, C 6alkyl, C 6alkyloxy, trifluoromethyl, cyano or hydroxycarbonyl.

In one aspect, the inhibitor of histone deacetylase activity may be a sulfonylamino derivative, such as, for example, compounds disclosed in WO2003076401, having the general formula:

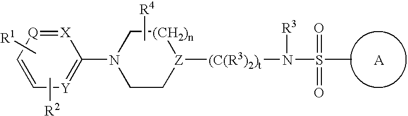

the N-oxide forms, the pharmaceutically acceptable addition salts and the stereo chemically isomeric forms thereof, wherein 1 n is 0, 1, 2 or 3 and when n is O then a direct bond is intended; t is 0, 1, 2, 3 or 4 and when t is O then a direct bond is intended; each Q is nitrogen or; _r each X is nitrogen or my; f each Y is nitrogen or; —CH— 2 each Z is nitrogen or ′;; R′ is —C(o)NR3R9, —N(H)C(O)R^{o}, —C(O)—C′ 6alkanediylSR′^{o}, —NRC(0)N(OH)R′^{o}, —NR″C(O)C 6alkanediylSR′^{o}, —NR″C(0)C—N(OH)R′^{o} or another Zn-chelating group wherein Rat and R9 are each independently selected from hydrogen, hydroxy, C′ 6alkyl, hydroxyC, 6alkyl, aminoC 6alkyl or aminoaryl; R^{o} is independently selected from hydrogen, Cal 6alkyl, C′ 6alkylcarbonyl, arylC, 6alkyl, Cal 6alkylpyrazinyl, pyridinone, pyrrolidinone or methylimidazolyl;: R′ is independently selected from hydrogen or C, 6alkyl; R2 is hydrogen, halo, hydroxy, amino nitro, Cal 6alkyl, Cal 6alkyloxy, trifluoromethyl, di(C-6alkYl)amino, hydroxyamino or naphtalenylsulfonylpyrazinyl; each R3 independently represents a hydrogen atom and one hydrogen atom can be replaced by a substituent selected from aryl; R4 is hydrogen, hydroxy, amino, hydroxyCI 6alkyl, C1 6alkyl, C1 6alkyloxy, arylC1 6alkyl, aminocarbonyl, hydroxycarbonyl, aminoCI 6alkyl, aminocarbonylCI 6alkyl, hydroxycarbonylC 6alkyl, hydroxyaminocarbonyl, C1 6alkyloxycarbonyl, C1 6alkylaminoC1 6alkyl or di(C 6alkyl)aminoCI 6alkyl; Rs is hydrogen, C1 6alkyl, C3 locycloalkyl, hydroxyCI 6alkyl, C1 6alkyloxyC1 6alkyl, di(CI 6alkyl)aminoC1 6alkyl or aryl; &t;) is a radical selected from jR6)s fR6)s fR7)sjR7)s [ ¢)N (a-1) (a-2) (a-3)(a) jR7)s jR7)s jR7)s H R7)s N:: NH: (a-5) (a-6) (a-7) (a-8) (R7)5 jR7)s fR7)s fR7)5 (a-9) (a-10) (a-11) (a-12) iN S S (a-13) (a-14) (a-15) (a-16)-79 CH 7 R7) N (a-17) (a-18)==N (a-19) N (a-20) 1 6)s iR7)s JR7)s fR7)s N′ (a-21) (a-22) (a-23) (a-24) JR7)s fR7)s fR6)s fR7)s IN &t; (a-25) (a-26) (a-27) (a-28) iR7)s JR7)s JR7)s jR7)s H IN ¢) (a-29) (a-30) (a-31) (a-32) (R) FIR) s SIR)s )s H 10 (a-33) (a-34) (a-35) (a-36) jR7) 7)s iR7)s fR7)s H (a-37) (a-38) (a-3g) (a-40)-80 iR7)s jR7)s iR6)sjR7)s N 60N.: (a-41) (a-42) (a-43) (a-44) K7)s^{o}R7)s^{o}R7)s fR7)s /(NH /36) N (a-45) (a-46) (a-47) (a-48) 1 R7)s fR7)s fR7)s 1, NH (a-49) (a-50) (a-51) wherein each s is independently 0, 1, 2, 3, 4 or 5; each R6 and R7 are independently selected from hydrogen; halo; hydroxy; amino; nitro; trihaloC 6alkyl; trihaloC 6alkyloxy; C 6alkyl; C 6alkyl substituted with aryl and C3 0cycloalkyl; C 6alkyloxy; C 6alkyloxyC 6alkyloxy; Ci 6alkylcarbonyl; 1 C 6alkyloxycarbonyl; C 6alkylsulfonyl; cyanoC 6alkyl; hydroxyC 6alkyl; hydroxyC 6alkyloxy; hydroxyC 6alkylamino; aminoC 6alkyloxy; di(C 6alkyl)aminocarbonyl; di(hydroxyC 6alkyl)amino; (aryl)(C 6alkyl)amino; di(C 6alkyl)aminoC 6alkyloxy; di(C 6alkyl)aminoC 6alkylamino; di(Cj 6alkyl)aminoC 6alkylaminoC 6alkyl; arylsulfonyl; arylsulfonylamino; aryloxy; aryloxyC 6alkyl; arylC2 6alkenediyl; di(C 6alkyl)amino; di(C 6alkyl)aminoC 6alkyl; di(C 6alkyl)amino(C 6alkyl)amino; di(C-6alkyl)amino(C 6alkyl)aminoC 6alkyl; di(C 6alkyl)aminoC 6alkyl(C 6alkyl)amino; di(C 6alkyl)aminoC 6alkyl(C 6alkyl)aminoC 6alkyl; 2 aminosulfonylamino(C 6alkyl)amino; aminosulfonylamino(C-6alkyl)aminoC 6alkyl; di(C 6alkyl)aminosulfonylamino(C 6alkyl)amin6; di(C-6alkyl)aminosulfonylamino(C-6alkyl)aminoC 6alkyl; cyano; thiophenyl; thiophenyl substituted with di(C 6alkyl)aminoC 6alkyl(C 6alkyl)aminoC 6alkyl, di(C 6alkyl)aminoC 6alkyl, C 6alkylpiperazinylC 6alkyl, hydroxyC 6alkylpiperazinylC 6alkyl, hydroxyC 6alkyloxyC 6alkylpiperazinylC 6alkyl, di(CI 6alkyl)aminosulfonylpiperazinylC 1 6alkyl, C I-6alkyloxypiperidinyl, C1 6alkyloxypiperidinylCI-6alkyl′ morpholinylCI 6alkyl, hydroxyCI 6alkyl(C1 6alkyl)aminoC1 6alkyl, or di(hydroxyCI 6alkyl)aminoC1 6alkyl; furanyl; furanyl substituted with hydroxyCI 6alkyl; benzofuranyl; imidazolyl; oxazolyl; oxazolyl substituted with aryl and C1 6alkyl; C 6alkyltriazolyl; tekazolyl; pyrrolidinyl; pyrrolyl; piperidinylC 6alkyloxy; morpholinyl; C1 6alkylmorpholinyl; morpholinylCI 6alkyloxy; morpholinylCI 6alkyl; morpholinylCI 6alkylamino; morpholinylCI 6alkylaminoC1 6alkyl; piperazinyl; C1 6alkylpiperazinyl; C1 6alkylpiperazinylC1 6alkyloxy; piperazinylCI 6alkyl; 1 naphtalenylsulfonylpiperazinyl; naphtalenylsulfonylpiperidinyl; naphtalenylsulfonyl; CI 6alkylpiperazinylC1 6alkyl; C1 6alkylpiperazinylC 6alkylamino; CI-6alkylpiperazinylCI 6alkylaminoC1 6alkyl; C1 6alkylpiperazinylsulfonyl; aminosulfonylpiperazinylCI 6alkyloxy; aminosulfonylpiperazinyl; aminosulfonylpiperazinylCI 6alkyl; di(CI 6alkyl)aminosulfonylpiperazinyl; di(CI 6alkyl)aminosulfonylpiperazinylC1 6alkyl; hydroxyCI 6alkylpiperazinyl; hydroxyCI 6alkylpiperazinylCI 6alkyl; C1 6alkyloxypiperidinyl; C-6alkyloxypiperidinylC I 6alkyl; piperidinylaminoC-6alkylamino; piperidinylaminoC I-6alkylaminoc 1-6alkYl; (CI 6alkylpiperidinyl)(hydroxyC′ 6alkyl)aminoC1 6alkylamino; 2 (C1 6alkylpiperidinyl)(hydroxyC, 6alkyl)aminoC1 6alkylaminoC1 6alkyl; hydroxyCI 6alkyloxyCI-6alkylpiperazinyl; hydroxyC 6alkyloxyCI 6alkylpiperazinylC1 6alkyl; (hydroxyCI 6alkyl)(CI 6alkyl)amino; (hydroxyC 6alkyl)(CI 6alkyl)aminoCI 6alkyl; hydroxyCI-6alkylaminoCI6alkyl; di(hydroxyCI-6alkyl)aminoC I 6alkyl; pyrrolidinylCI 6alkyl; pyrrolidinylCI 6alkyloxy; pyrazolyl; thiopyrazolyl; pyrazolyl substituted with two substituents selected from C1 6alkyl or trihaloCI 6alkyl; pyridinyl; pyridinyl substituted with C1 6alkyloxy, aryloxy or aryl; pyrimidinyl; tetrahydropyrimidinylpiperazinyl; tetrahydropyrimidinylpiperazinylCI 6alkyl; quinolinyl; indole; phenyl; phenyl substituted with one, two or three substituents independently selected from halo, amino, nitro, C1 6alkyl, C1 6alkyloxy, hydroxyCI 4alkyl, trifluoromethyl, trifluoromethyloxy, hydroxyCI 4alkyloxy, C1 4alkylsulfonyl, C1 4alkyloxyCI 4alkyloxy, C1 4alkyloxycarbonyl, aminoCI 4alkyloxy, di(CI 4alkyl)aminoCI 4alkyloxy, di(C1 4alkyl)amino, di(CI 4alkyl)aminocarbonyl, di(CI 4alkyl)aminoCI 4alkyl, 35 di(CI 4alkyl)aminoCI 4alkylaminoCI 4alkyl, di(CI 4alkyl)amino(CI 4alkyl)amino, di(C 4alkyl)amino(CI 4alkyl)aminoC1 4alkyl, di(C 4alkyl)aminoC1 4alkyl(C1 4alkyl)amino, di(C 4alkyl)aminoC 4alkyl(C 4alkyl)aminoCI 4alkyl, aminosulfonylamino(C 4alkyl)amino, aminosulfonylamino(C 4alkyl)aminoC 4alkyl, di(C 4alkyl)aminosulfonylamino(C 4alkyl)amino, di(C 4alkyl)aminosulfonylamino(C 4alkyl)aminoC 6alkyl, cyano, piperidinylC 4alkyloxy, pyrrolidinylC 4alkyloxy, aminosulfonylpiperazinyl, aminosulfonylpiperazinylC 4alkyl, di(C 4alkyl)aminosulfonylpiperazinyl, di(C 4alkyl)aminosulfonylpiperazinylC 4alkyl, hydroxyC 4alkylpiperazinyl, hydroxyC 4alkylpiperazinylC 4alkyl, C 4alkyloxypiperidinyl, C 4alkyloxypiperidinylC 4alkyl, hydroxyC 4alkyloxyC 4alkylpiperazinyl, 1 hydroxyC 4alkyloxyC 4alkylpiperazinylC 4alkyl, (hydroxyC 4alkyl)(C 4alkyl)amino, (hydroxyc 4alkyl)(C 4alkyl)aminoC 4alkyl, di(hydroxyC 4alkyl)amino, di(hydroxyC 4alkyl)aminoC 4alkyl, furanyl, furanyl substituted with —CH═CH—CH═CH—, pyrrolidinylC 4alkyl, pyrrolidinylC 4alkyloxy, morpholinyl, morpholinylC 4alkyloxy, morpholinylC 4alkyl, morpholinylC 4alkylamino, morpholinylC 4alkylaminoC 4alkyl, piperazinyl, C 4alkylpiperazinyl, C 4alkylpiperazinylC 4alkyloxy, piperazinylC 4alkyl, C 4alkylpiperazinylC 4alkyl, C 4alkylpiperazinylC 4alkylamino, C 4alkylpiperazinylC 4alkylaminoC 6alkyl, tetrahydropyrimidinylpiperazinyl, tetrahydropyrimidinylpiperazinylC 4alkyl, piperidinylaminoC 4alkylamino, piperidinylaminoC 4alkylaminoC 4alkyl, (C 4alkylpiperidinyl)(hydroxyC, 4alkyl)aminoC 4alkylamino, (C 4alkylpiperidinyl)(hydroxyC, 4alkyl)aminoC 4alkylaminoC 4alkyl, pyridinylC 4alkyloxy, hydroxyC 4alkylamino, hydroxyC 4alkylaminoC 4alkyl, di(C 4alkyl)aminoC 4alkylamino, aminothiadiazolyl, aminosulfonylpiperazinylC 4alkyloxy, or thiophenylC 4alkylamino; each R6 and R7 can be placed on the nitrogen in replacement of the hydrogen; aryl in the above is phenyl, or phenyl substituted with one or more substituents each independently selected from halo, C, 6alkyl, C′ 6alkyloxy, trifluoromethyl, cyano or 30 hydroxycarbonyl.

In one aspect, the inhibitor of histone deacetylase activity may be a compound of the general formula:

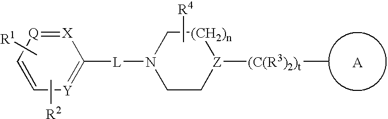

the N-oxide forms, the pharmaceutically acceptable addition salts and the stereo-chemically isomeric forms thereof, wherein 10 n is 0, 1, 2 or 3 and when n is 0 then a direct bond is intended; t is 0, 1, 2, 3 or 4 and when t is 0 then a direct bond is intended; each Q is nitrogen or Hi; 15 i: -r; each X is nitrogen or my; each Y is nitrogen or Be; —CH-20; each Z is nitrogen or′; Rat is —C(o)NR7R8, —NHC(0)R9, —C(O)—C 6alkanediylSR9, —NROC(o)N(oH)R9, —NRi^{o}C(O)C 6alkanediylSR9, —NROC(o)C═N(oH)R9 or another Zn-chelating group wherein R7 and Rig are each independently selected from hydrogen, hydroxy, Cal 6alkyl, hydroxyC 6alkyl, amino 6alkyl or aminoaryl; R9 is independently selected from hydrogen, Cal 6alkyl, Cal 6alkylcarbonyl, arylC 6alkyl, Cal 6alkylpyrazinyl, pyridinone, pyrrolidinone or methylimidazolyl; Ri^{o} is independently selected from hydrogen or Cat 6alkyl; R2 is hydrogen, halo, hydroxy, amino, nitro, Cat 6alkyl, Cat 6alkyloxy, trifluoromethyl, ditch 6alkyl)amino, hydroxyamino or naphtalenylsulfonylpyrazinyl; -L- is a direct bond or a bivalent radical selected from Cat 6alkanediyl, Cat 6alkanediyloxy, amino, carbonyl or aminocarbonyl; each R3 independently represents a hydrogen atom and one hydrogen atom can be replaced by a substituent selected from aryl; R4 is hydrogen, hydroxy, amino, hydroxyC1 6alkyl, C1 6alkyl, C1 6alkyloxy, 1 arylC1 6alkyl, aminocarbonyl, hydroxycarbonyl, aminoC1 6alkyl, I aminocarbonylC1 6alkyl, hydroxycarbonylC1 6alkyl, hydroxyaminocarbonyl, C1 6alkyloxycarbonyl, C1 6alkylaminoC1 6alkyl or di(C1 6alkyl)aminoC1 6alkyl;) is a radical selected from jR)s iRs)s jR)s jR)s N (a-1) (a-2) (a-3? (a-4); /(s CR6)S /(R6)S /(R6) N; H; (a-5) (a-6) (a-7) (a-8!. tR6)R6)s JR)s ′/4′, , , (a-9) (a-.10) (a-) (a-12): ; S g;)5) . . . (a-13) (a-14) (a-15) (a-16) ::CtR is (a-17) (a-18) ==N(a-19) N (a-20) 1 1 55)s 1 6)S 6)s 6)s :[: t′; , (a-21) ta-22) (a-23) (a-24) ′.″: )s 6))s 6). IN GINO (a-25) (a-26) (a-27) (a-28): , If, /6)S,)s)s, ′JR6)S ′:′″&t; ″. H (a-29) (a-30) (a-31) (a-32) OR6)s OR)s O)S )s | NO IN-1: 3 &t;No 1 (a-33) (a-34) (a-35). (a-36) )s 6)s 6)s 6)s H (a-37) (a-38) (a 39? (a-40) JR6)s IR6)s JR5)s fR6)s /Ni N (a-41) (a-42) (a-43) (a-44) OiR6)S, OR6)S O /6)S IR6)s (NH N3 C (a-45) ′(a-46) (a-47) (a-48):(R6) ′(R6) ′R6 (a-49) (a-50j(a-51):: -: wherein each s is independently 0, 1, 2, 3, 4 or 5; each Rs and R6 are independently selected from hydrogen; halo; hydroxy; amino; nitro; trihaloC1 6alkyl; trihaloC 6alkyloxy; C1 6alkyl; C1 6alkyl substituted with aryl and 10 C3 0cycloalkyl; C1 6alkyloxy; C1 6alkyloxyC1 6alkyloxy; C1 6alkylcarbonyl; C 6alkyloxycarbonyl, C1 6alkylsulfonyl; cyanoC1 6alkyl; hydroxyC1 6alkyl; hydroxyC1 6alkyloxy; hydroxyC1 6alkylamino; aminoC1 6alkyloxy; di(CI 6alkyl)aminocarbonyl; di(hydroxyCI 6alkyl)amino; (aryl)(C1 6alkyl)amino; di(CI 6alkyl)aminoC1 6alkyloxy; di(CI 6alkyl)aminoC1 6alkylamino; di(C 6alkyl)aminoC1 6alkylaminoC1 6alkyl; arylsulfonyl; arylsulfonylamino; aryloxy; aryloxyCI 6alkyl; arylC2 6alkenediyl; di(CI 6alkyl)amino; di(C1 6alkyl)aminoC1 6alkyl; di(C1 6alkyl)amino(C1 6alkyl)amino, di(C1 6alkyl)amino(C1 6alkyl)aminoC1 6alkyl; di(C 6alkylaminoCI 6alkyl(C1 6alkyl)amino; 2 di(C1 6alkyl)aminoCI 6alkyl(C1 6alkyl)aminoC1 6alkyl; aminosulfonylamino(C1 6alkyl)amino; ′aminosulfonylamino(C1 6alkyl)aminoC1 6alkyl; di(C1 6alkyl)aminosulfonylamino(C1 6alkyl)amino; di(C1 6alkyl)aminosulfonylamino(C1 6alkyl)aminoC 6alkyl; cyano; thiophenyl; thiophenyl substituted with di(C 6alkyl)aminoC1 6alkyl(C1 6alkyl)aminoC 6alkyl, di(C1 6alkyl)aminoC 6alkyl, C1 6alkylpiperazinylC 6alkyl, hydroxyC1 6alkylpiperazinylC 6alkyl, hydroxyC1 6alkyloxyC1 6alkylpiperazinylC1 6alkyl, di(C1 6alkyl)aminosulfonylpiperazinylCI 6alkyl, C1 6alkyloxypiperidinyl, C1 6alkyloxypiperidinylC1 6alkyl, morpholinylC1 6alkyl, hydroxyC1 6alkyl(C1 6alkyl)aminoC1 6alkyl, or di(hydroxyC1 6alkyl)aminoC1 6alkyl; 5 furanyl; furanyl substituted with hydroxyC1 6alkyl; benzofuranyl; imidazolyl; oxazolyl; oxazolyl substituted with aryl and C1 6alkyl; C1 6alkyltriazolyl; tetrazolyl; pyrrolidinyl; pyrrolyl; piperidinylC1 6alkyloxy; morpholinyl; C1 6alkylmorpholinyl; morpholinylC1 6alkyloxy; morpholinylC1 6alkyl; morpholinylC1 6alkylamino; morpholinylC1 6alkylaminoC1 6alkyl; piperazinyl; C1 6alkylpiperazinyl; C1 6alkylpiperazinylC1 6alkyloxy; piperazinylC1 6alkyl; naphtalenylsulfonylpiperazinyl; naphtalenylsulfonylpiperidinyl; naphtalenylsulfonyl; C1 6alkylpiperazinylC1 6alkyl; C1 6alkylpiperazinylC1 6alkylamino; C1 6alkylpiperazinylC1 6alkylaminoC1 6alkyl; C1 6alkylpiperazinylsulfonyl; aminosulfonylpiperazinylC1 6alkyloxy; aminosulfonylpiperazinyl; aminosulfonylpiperazinylC1 6alkyl; di(C1 6alkyl)aminosulfontylpiperazinyl; ′di(C1 6alkyl)aminosulfonylpiperazinylC1 6alkyl; hydroxyC1 6alkylpiperazinyl; hydroxyC1 6alkylpiperazinylC1 6alkyl; C1 6alkyloxypiperidinyl; C1 6alkyloxypiperidinylC1 6alkyl; piperidinylaminoC1 6alkylamino; 2 piperidinylaminoC1 6alkylaminoC1 6alkyl; (C1 6alkylpiperidinyl)(hydroxyCI 6alkyl)aminoC1 6alkylamino; (C1 6alkylpiperidinyl)(hydroxyCI 6alkyl)aminoC1 6alkylaminoC1 6alkyl; hydroxyC1 6alkyloxyC1 6alkylpiperazinyl; hydroxyC1 6alkyloxyC1 6alkylpiperazinylC1 6alkyl; (hydroxyC1 6alkyl)(C1 6alkyl)amino; (hydroxyC1 6alkyl)(C1 6alkyl)aminoC1 6alkyl; hydroxyC1 6alkylaminoC1 6alkyl; di(hydroxyC1 6alkyl)aminoC1 6alkyl; pyrrolidinylC1 6alkyl; pyrrolidinylC1 6alkyloxy; pyrazolyl; thiopyrazolyl; pyrazolyl substituted with two substituents selected from C1 6alkyl or trihaloC1 6alkyl; pyridinyl; pyridinyl substituted with C1 6alkyloxy, aryloxy or aryl; pyrimidinyl; tetrahydropyrimidinylpiperazinyl; tetrahydropyrimidinylpiperazinylC1 6alkyl; quinolinyl; indole; phenyl; phenyl substituted with one, two or three substituents independently selected from halo, amino, nitro, C1 6alkyl, C1 6alkyloxy, hydroxyC1 4alkyl, trifluoromethyl, trifluoromethyloxy, hydroxyC1 4alkyloxy, C1 4alkylsulfonyl, C1 4alkyloxyC1 4alkyloxy, C1 4alkyloxycarbonyl, aminoC1 4alkyloxy, di(C1 4alkyl)aminoC1 4alkyloxy, di(C1 4alkyl)amino, di(C1 4alkyl)aminocarbonyl, di(C1 4alkyl)aminoC 4alkyl, di(C 4alkyl)aminoC1 4alkylaminoC1 4alkyl, di(C 4alkyl)amino(C1 4alkyl)amino, di(C1 4alkyl)amino(C1 4alkyl)aminoC1 4alkyl, di(C1 4alkyl)aminoC1 4alkyl(C1 4alkyl)amino, di(C1 4alkyl)aminoC1 4alkyl(C1 4alkyl)aminoC1 4alkyl, aminosulfonylamino(C1 4alkyl)amino, aminosulfonylamino(C1 4alkyl)aminoC1 4alkyl, di(C1 4alkyl)aminosulfonylamino(C 4alkyl)amino, di(C1 4alkyl)aminosulfonylamino(C 4alkyl)aminoC 6alkyl, cyano, piperidinylC1 4alkyloxy, pyrrolidinylC 4alkyloxy, aminosulfonylpiperazinyl, aminosulfonylpiperazinylC 4alkyl, di(C1 4alkyl)aminosulfonylpiperazinyl, di(C 4alkyl)aminosulfonylpiperazinylC1 4alkyl, hydroxyC1 4alkylpiperazinyl, 1 hydroxyC1 4alkylpiperazinylC1 4alkyl, C1 4alkyloxypiperidinyl, C1 4alkyloxypiperidinylC1 4alkyl, hydroxyCI 4alkyloxyC1 4alkylpiperazinyl, hydroxyC1 4alkyloxyC1 4alkylpiperazinylC1 4alkyl, (hydroxyC 4alkyl)(C 4alkyl)amino, (hydroxyC1 4alkyl)(C1 4alkyl)aminoc1-4alkyl, di(hydroxyC1 4alkyl)amino, di(hydroxyC1 4alkyl)aminoC1 4alkyl, furanyl, furanyl substituted with —CH—CH—CH═CH—, pyrrolidinylC14alkyl, pyrrolidinylC1 4alkyloxy, morpholinyl, morpholinylC1 4alkyloxy, morpholinylC1 4alkyl, morpholinylC 4alkylamino, morpholinylC 4alkylaminoC1 4alkyl, piperazinyl, C1 4alkylpiperazinyl, C 4alkylpiperazinylC1 4alkyloxy, piperazinylC1 4alkyl, C 4alkylpiperazinylC1 4alkyl, C 4alkylpiperazinylC 4alkylamino, 2 C 4alkylpiperazinylC1 4alkylaminoC1 6alkyl, tetrahydropyrimidinylpiperazinyl, tetrahydropyrimidinylpiperazinylC1 4alkyl, piperidinylaminoC 4alkylamino, piperidinylaminoC 4alkylaminoC 4alkyl, (C1 4alkylpiperidinyl)(hydroxyC 4alkyl)aminoC1 4alkylamino, (c1-4alkylpiperidinyl)(hydroxyC-4alkyl)aminoc2-4alkylaminoc1-4alkyl, pyridinylC1 4alkyloxy, hydroxyC1 4alkylamino, hydroxyC1 4alkylaminoC, 4alkyl, di(C1 4alkyl)aminoC 4alkylamino, aminothiadiazolyl, aminosulfonylpiperazinylC1 4alkyloxy, or thiophenylC1 4alkylamino; each R5 and R6 can be placed on the nitrogen in replacement of the hydrogen; aryl in the above is phenyl, or phenyl substituted with one or more substituents each independently selected from halo, C 6alkyl, C1 6alkyloxy, trifluoromethyl, cyano or hydroxycarbonyl. See, for example, compounds disclosed in WO2003076400.

In one aspect, the inhibitor of histone deacetylase activity may be an aminocarbonyl derivative, such as, for example, compounds disclosed in WO2003076421, having the general formula:

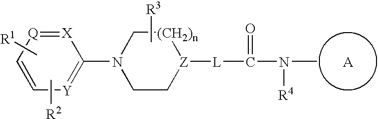

the N-oxide forms, the pharmaceutically acceptable addition salts and the stereo-chemically isomeric forms thereof, wherein I 10 n is 0, 1, 2 or 3 and when n is O then a direct bond is intended; each Q is nitrogen or; each X is nitrogen or; each Y is nitrogen or; —CH— each Z is nitrogen or′; R1 is —C(O)NR7Rs, —NCH)C(O)R9, —C(O)—C6alkanediylSR9, —NRI^{o}C(O)N(OH)R9, —N Ri^{o}C(O)Ci6alkanediylSR9, —NR^{o}C(O)C═N(OH)R9 or another Zn-chelating group wherein R7 and Ret are each independently selected from hydrogen, hydroxy, Cal 6alkyl, hydroxyC 6alkyl, amino 6alkyl or aminoaryl; 2 R9 is independently selected from hydrogen, Cal 6alkyl, Cal 6alkylcarbonyl, arylC 6alkyl, Cal 6alkylpyrazinyl, pyridinone, pyrrolidinone or methylimidazolyl; Skis independently selected from hydrogen or Cat 6alkyl; R2 is hydrogen, halo, hydroxy, amino, nitro, Cat 6alkyl, Cal 6alkyloxy, trifluoromethyl, di(C 6alkyl)amino, hydroxyamino or naphtalenylsulfonylpyrazinyl; R3 is hydrogen, hydroxy, amino, hydroxyC 6alkyl, Cat 6alkyl, C at 6alkyloxy, Dryly 6alkyl, aminocarbonyl, hydroxycarbonyl, aminoC 6alkyl, aminocarbonylC 6alkyl, hydroxycarbonylC 6alkyl, hydroxyaminocarbonyl, Cat 6alkyloxycarbonyl, Cat 6alkylaminoC 6alkyl or di(C 6alkyl)aminoC 6alkyl; when Z is equal to nitrogen, then -L- is a direct bond; when Z is equal to′, then -L- is —NH— or the bivalent radical —C1 6alkanedlylNH—; i R4 is hydrogen, C1 6alkyl, C3 Ocycloalkyl, hydroxyC1 6alkyl, C1 6alkyloxyC1 6alkyl, di(C1 6alkyl)aminoC1 6alkyl or aryl; is a radical selected from iR5)s jR5)s fR6)s)s IN (a-1) (a-2) (a-3) (a-4) )S)S 6)s 6) (a-S)(a-6) (a-7) (a-g) 6)sjR6)s P6) s fR6) IN (a-9)(a-10) (a-11) (a-12): 5′(a-13)(a-14) (a-15) (a-16)-49 H CN″&t; N::)s)s (a-17) (a-18) t==N (a-19) N (a-20) 1 IR5)S jR6) s JR6)s fR6)s (a-21) (a-22) (a-23) (a-24) 6)s R6)s Rs)s 6) o H (a-25) (a-26) (a-27) (a-28) jR6)s fR6)s fR6)s jR6 H i) IN to&t; (a-29) (a-30) (a-31) (a-32) 6,s 1 6)S o (R6) 6)s IN N N) &t; N H 10 (a-33) (a-34) (a-35) (a-36) jR6) fR6) iR6) 6)S I No NO (a-37) (a-38) (a-39) (a-40) -50 fR6)s iR6)sJR5)s fR6)s I:/′″fq /lfq′f/ N: NJ (a-41) (a-42)(a-43) (a-44) O IR6)S O JR6)SO P6)S JR6)s /(NH Nt /3N /X $6Q (a-45) (a-46) (a-47) (a-48) 1 fR6)S IR6)s)s [: N:1 5 (a-49) (a-SO)(a-51) wherein each s is independently 0, 1, 2, 3, 4 or 5; each R5 and R6 are independently selected from hydrogen; halo; hydroxy; amino; nitro; trihaloC 6alkyl; trihaloC 6alkyloxy; C 6alkyl; C 6alkyl substituted with aryl and C3 0cycloalkyl; C 6alkyloxy; C 6alkyloxyC 6alkyloxy; C 6alkylcarbonyl; 10 C 6alkyloxycarbonyl; C 6alkylsulfonyl; cyanoC 6alkyl; hydroxyC 6alkyl; hydroxyC 6alkyloxy; hydroxyC 6alkylamino; aminoC 6alkyloxy; di(C 6alkyl)aminocarbonyl; di(hydroxyC 6alkyl)amino; (aryl)(C 6alkyl)amino; di(C 6alkyl)aminoC 6alkyloxy; di(Ci 6alkyl)aminoC 6alkylamino; di(C 6alkyl)aminoC 6alkylaminoC 6alkyl; arylsulfonyl; arylsulfonylamino; 15 aryloxy; aryloxyC 6alkyl; arylC2 6alkenediyl; di(C 6alkyl)amino; di(C 6alkyl)aminoC 6alkyl; di(C 6alkyl)amino(C 6alkyl)amino; di(C 6alkyl)amino(C 6alkyl)aminoC 6alkyl; di(Ci 6alkyl)aminoC6alkyl(C 6alkyl)amino; di(C 6alkyl)aminoC 6alkyl(C 6alkyl)aminoC 6alkyl; aminosulfonylamino(C 6alkyl)amino; aminosulfonylamino(C 6alkyl)aminoC 6alkyl; di(Ci 6alkyl)aminosulfonylamino(C 6alkyl)amino; di(C 6alkyl)aminosulfonylamino(C 6alkyl)aminoC 6alkyl; cyano; thiophenyl; thiophenyl substituted with di(C 6alkyl)aminoC 6alkyl(C 6alkyl)aminoC 6alkyl, 2 di(C 6alkyl)aminoC 6alkyl, C 6alkylpiperazinylC 6alkyl, hydroxyC 6alkylpiperazinylC 6alkyl, hydroxyc-6alkyloxyc 6alkylpiperazinylC 6alkyl, di(C1 6alkyl)aminosulfonylpiperazinylC1 6alkyl, C1 6alkyloxypiperidinyl, C1 6alkyloxypiperidinylC1 6alkyl, morpholinylC1 6alkyl, hydroxyC1 6alkyl(C1 6alkyl)aminoC1 6alkyl, or di(hydroxyC1 6alkyl)aminoC1 6alkyl; furanyl; furanyl substituted with hydroxyC1 6alkyl; benzofuranyl; imidazolyl; S oxazolyl; oxazolyl substituted with aryl and C 6alkyl; C1 6alkyltriazolyl; tetrazolyl; pyrrolidinyl; pyrrolyl; piperidinylC1 6alkyloxy; morpholinyl; C1 6alkylmorpholinyl; morpholinylC1 6alkyloxy; morpholinylC1 6alkyl; morpholinylC1 6alkylamino; morpholinylC1 6alkylaminoC1 6alkyl; piperazinyl; C1 6alkylpiperazinyl; C1 6alkylpiperazinylC1 6alkyloxy; piperazinylC1 6alkyl; naphtalenylsulfonylpiperazinyl; naphtalenylsulfonylpiperidinyl; naphtalenylsulfonyl: C1 6alkylpiperazinylC1 6alkyl; C1 6alkylpiperazinylC 6alkylamino; C1 6alkylpiperazinylC1 6alkylaminoC1 6alkyl; C1 6alkylpiperazinylsulfonyl; aminosulfonylpiperazinylC1 6alkyloxy; aminosulfonylpiperazinyl; aminosulfonylpiperazinylC1 6alkyl; di(C1 6alkyl)aminosulfonylpiperazinyl; di(C1 6alkyl)aminosulfonylpiperazinylC1 6alkyl; hydroxyC1 6alkylpiperazinyl; hydroxyC1 6alkylpiperazinylC1 6alkyl; C1 6alkyloxypiperidinyl; C1 6alkyloxypiperidinylC1 6alkyl; piperidinylaminoC1 6alkylamino; piperidinylaminoC1 6alkylaminoC1 6alkyl; (C1 6alkylpiperidinyl)(hydroxyC 6alkyl)aminoC1 6alkylamino; (C1 6alkylpiperidinyl)(hydroxyC 6alkyl)aminoC1 6alkylaminoC1 6alkyl; hydroxyC1 6alkyloxyC1 6alkylpiperazinyl; hydroxyC1 6alkyloxyC1 6alkylpiperazinylC1 6alkyl; (hydroxyC1 6alkyl)(C1 6alkyl)amino; (hydroxyC1 6alkyl)(CI 6alkyl)aminoC1 6alkyl; 25 hydroxyC1 6alkylaminoC1 6alkyl; di(hydroxyC1 6alkyl)aminoC1 6alkyl; pyrrolidinylC 6alkyl; pyrrolidinylC1 6alkyloxy; pyrazolyl; thiopyrazolyl; pyrazolyl substituted with two substituents selected from C1 6alkyl or trihaloC1 6alkyl; pyridinyl; pyridinyl substituted with C1 6alkyloxy, aryloxy or aryl; pyrirnidinyl; tetrahydropyrimidinylpiperazinyl; tetrahydropyrimidinylpiperazinylC1 6alkyl; quinolinyl; indolyl; phenyl; phenyl substituted with one, two or three substituents independently selected from halo, amino, nitro, C1 6alkyl, C1 6alkyloxy, hydroxyC1 4alkyl, trifluoromethyl, trifluoromethyloxy, hydroxyC1 4alkyloxy, C1 4alkylsulfonyl, C1 4alkyloxyC1 4alkyloxy, C1 4alkyloxycarbonyl, aminoC1 4alkyloxy, di(C1 4alkyl)aminoC1 4alkyloxy, di(C1 4alkyl)amino, 3 di(C1 4alkyl)aminocarbonyl, di(C1 4alkyl)aminoC1 4alkyl, di(C1 4alkyl)aminoC1 4alkylaminoC1 4alkyl, di(C1 4alkyl)amino(C1 4alkyl)amino, di(C1 4alkyl)amino(C1 4alkyl)aminoC1 4alkyl, di(C1 4alkyl)aminoC1 4alkyl(C1 4alkyl)amino, di(C1 4alkyl)aminoC1 4alkyl(C1 4alkyl)aminoC1 4alkyl, aminosulfonylamino(C1 4alkyl)amino, aminosulfonylamino(C1 4alkyl)aminoC1 4alkyl, di(C1 4alkyl)aminosulfonylamino(C1 4alkyl)amino, di(C1 4alkyl)aminosulfonylamino(C1 4alkyl)aminoC1 6alkyl, cyano, piperidinylCI 4alkyloxy, pyrrolidinylCI 4alkyloxy, aminosulfonylpiperazinyl, aminosulfonylpiperazinylC1 4alkyl, di(C1 4alkyl)aminosulfonylpiperazinyl, di(C1 4alkyl)aminosulfonylpiperazinylC1 4alkyl, hydroxyC1 4alkylpiperazinyl, hydroxyC1 4alkylpiperazinylC1 4alkyl, C1 4alkyloxypiperidinyl, C1 4alkyloxypiperidinylC1 4alkyl, hydroxyCI 4alkyloxyCI 4alkylpiperazinyl, hydroxyC1 4alkyloxyC1 4alkylpiperazinylC1 4alkyl, (hydroxyC1 4alkyl)(C1 4alkyl)amino, (hydroxyC1 4alkyl)(C1 4alkyl)aminoC1 4alkyl, di(hydroxyC1 4alkyl)amino, di(hydroxyC1 4alkyl)aminoC1 4alkyl, furanyl, furanyl substituted with —CH═CH—CH═CH—, pyrrolidinylC1 4alkyl, pyrrolidinylC1 4alkyloxy, morpholinyl, morpholinylC1 4alkyloxy, morpholinylC1 4alkyl, morpholinylC1 4alkylamino, morpholinylC1 4alkylaminoC1 4alkyl, piperazinyl, C1 4alkylpiperazinyl, C1 4alkylpiperazinylC1 4alkyloxy, piperazinylC1 4alkyl, C1 4alkylpiperazinylC1 4alkyl, C1 4alkylpiperazinylC1 4alkylamino, C1 4alkylpiperazinylC1 4alkylaminoC1 6alkyl, tetrahydropyrimidinylpiperazinyl, tetrahydropyrimidinylpiperazinylC1 4alkyl, piperidinylaminoC1 4alkylamino, piperidinylaminoC1 4alkylaminoC1 4alkyl, (C1 4alkylpiperidinyl)(hydroxyC 4alkyl)aminoC1 4alkylamino, (C1 4alkylpiperidinyl)(hydroxyCI 4alkyl)aminoC1 4alkylaminoC1 4alkyl, pyridinylC1 4alkyloxy, 2 hydroxyCI 4alkylamino, hydroxyCI 4alkylaminoC1 4alkyl, di(C1 4alkyl)aminoC1 4alkylamino, aminothiadiazolyl, aminosulfonylpiperazinylCI 4alkyloxy, or thiophenylC1 4alkylamino; each Rs and R6 can be placed on the nitrogen in replacement of the hydrogen; aryl in the above is phenyl, or phenyl substituted with one or more substituents each independently selected from halo, C 6alkyl, C 6alkyloxy, trifluoromethyl, cyano or hydroxycarbonyl.

In one aspect, the inhibitor of histone deacetylase activity may be a compound having the general formula:

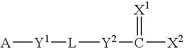

wherein A is a cyclic moiety selected from the group consisting of C3_{—}14 cycloalkyl, 3-14 membered heterocycloalkyl, C4_{—}14 cycloalkenyl, 3-8 membered heterocycloalkenyl, aryl, or heteroaryl; the cyclic moiety being optionally substituted with alkyl, alkenyl, alkynyl, alkoxy, hydroxyl, hydroxylalkyl, halo, haloalkyl, amino, alkylcarbonyloxy, alkyloxycarbonyl, alkylcarbonyl, alkylsulfonylamino, aminosulfonyl, or alkylsulfonyl; or A is a saturated branched C3_{—}12 hydrocarbon chain or an unsaturated branched C3_{—}12 hydrocarbon chain optionally interrupted by —O—, —S—, —N(Ra)-, —C(O)—, —N(Ra)-SO2-, —SO2-N(Ra)-, —N(Ra)-C(O)—O—, —O—C(O)—N(Ra)-, —N(′a)-C(O)—N(Rb)-, —O—C(O)—, —C(O)—O—, —O—SO2-, —SO2-O—, or —O—C(O)—O—, where each of Ra and Rb, independently, is hydrogen, alkyl, alkenyl, alkynyl, alkoxy, hydroxylalkyl, hydroxyl, or haloalkyl; each of the saturated and the unsaturated branched hydrocarbon chain being optionally substituted with alkyl, alkenyl, alkynyl, alkoxy, hydroxyl, hydroxylalkyl, halo, haloalkyl, amino, alkylcarbonyloxy, alkyloxycarbonyl, alkylcarbonyl, alkylsulfonylamino, aminosulfonyl, or alkylsulfonyl; each of Y1 and Y2, independently, is —CH2-, -0-, —S—, —N(R)—, —N(R^{o})—C(O)-0-, —O—C(O)—N(R^{o})—, —N(Rc)-C(O)—N(Rd)-, —O—C(O)-0-, or a bond; each of R^{o} and Rd, independently, being hydrogen, alkyl, alkenyl, alkynyl, alkoxy, hydroxylalkyl, hydroxyl, or haloalkyl; L is a straight C2-12 hydrocarbon chain optionally containing at least one double bond, at least one triple bond, or at least one double bond and one triple bond; said hydrocarbon chain being optionally substituted with C1_{—}4 alkyl, C2_{—}4 alkenyl, C2_{—}4 alkynyl, C1_{—}4 alkoxy, hydroxyl, halo, amino, nitro, cyano, C3_{—}5 cycloalkyl, 3-5 membered heterocycloalkyl, monocyclic aryl, 5-6 membered heteroaryl, C1_{—}4 alkylcarbonyloxy, C₁_{—}4 alkyloxycarbonyl, CI-4 alkylcarbonyl, or formyl; and further being optionally interrupted by -0-, —N(Re)-, —N(Re)-C(O)-0-, —O—C(O)—N(Re)-, —N(Re)-C(O)—N(Rf)-, or —O—C(O)-0-; each of Wand R independently, being hydrogen, alkyl, alkenyl, alkynyl, alkoxy, hydroxylalkyl, hydroxyl, or haloalkyl; XI is O or S; and X2 is —OR′, —SRI, —NR′—OR′, —NR—SR′, —C(O)—OR′, —CHR4-OR′, —N═N—C(O)—N(R3)2, or -0-CHR4-O—C(O)—R5, where each of RI and R2, independently, is hydrogen, alkyl, hydroxylalkyl, haloalkyl, or a hydroxyl protecting group; R3 is hydrogen, alkyl, alkenyl, alkynyl, alkoxy, hydroxylalkyl, hydroxyl, haloalkyl, or an amino protecting group; R4 is hydrogen, alkyl, hydroxylalkyl, or haloalkyl; RS is alkyl, hydroxylalkyl, or haloalkyl; and provided that when L is a C2_{—}3 hydrocarbon containing no double bonds and X2 is —OR′, YI is not a bond and Y2 is not a bond; or a salt thereof.

### Therapeutic Use of the Cells of the Present Invention

In one aspect, the present invention provides a method for treating a patient suffering from, or at risk of developing Typel diabetes. This method involves isolating and culturing cells, expanding the isolated population of cells in vitro, differentiating the cultured cells into a β-cell lineage, or into a pancreatic hormone-secreting cell in vitro, and implanting the differentiated cells either directly or in a pharmaceutical carrier into the patient.

In yet another aspect, this invention provides a method for treating a patient suffering from, or at risk of developing Type 2 diabetes. The method involves isolating and culturing cells, expanding the isolated population of cells, differentiating the cultured cells into a β-cell lineage, or into a pancreatic hormone-secreting cell, in vitro and implanting the differentiated cells either directly or in a pharmaceutical carrier into said patient.

If appropriate, the patient may be further treated with pharmaceutical agents or bioactives that facilitate the survival and function of the transplanted cells. These agents may include, for example, insulin, members of the TGF-β family, including TGF-β1, 2, and 3, bone morphogenic proteins (BMP-2, -3, -4, -5, -6, -7, -11, -12, and -13), fibroblast growth factors-1 and -2, platelet-derived growth factor-AA, and -BB, platelet rich plasma, insulin growth factor (IGF-I, II) growth differentiation factor (GDF-5, -6, -8, -10, -15), vascular endothelial cell-derived growth factor (VEGF), pleiotrophin, endothelin, among others. Other pharmaceutical compounds can include, for example, nicotinamide, glucagon like peptide-I (GLP-1) and II, GLP-1 and 2 mimetibody, Exendin-4, retinoic acid, parathyroid hormone, MAPK inhibitors, such as, for example, compounds disclosed in US20040209901 and US20040132729.

The cells of the present invention may be genetically modified. For example, the cells may be engineered to over express markers characteristic of a cell of a β-cell lineage, such as, for example, NGN-3 (neurogenin-3),Pax-4, Pdx-1, Hlxb9, Nkx6, Isl-1, Pax6, NeuroD, HNF-1a, HNF-6, HNF-3 beta, and MafA, or insulin. The cells may be engineered to over express with any suitable gene of interest. Techniques useful to genetically modify the cells may be found, for example, in standard textbooks and reviews in cell biology. Methods in molecular genetics and genetic engineering are described, for example, in Molecular Cloning: A Laboratory Manual, 2nd Ed. (Sambrook et al., 1989); Oligonucleotide Synthesis (M. J. Gait, ed., 1984); Animal Cell Culture (R. I. Freshney, ed., 1987); the series Methods in Enzymology (Academic Press, Inc.); Gene Transfer Vectors for Mammalian Cells (I. M. Miller & M. P. Calos, eds., 1987); Current Protocols in Molecular Biology and Short Protocols in Molecular Biology, 3rd Edition (F. M. Ausubel et al., eds., 1987 & 1995); and Recombinant DNA Methodology II (R. Wu ed. , Academic Press 1995).

The nucleic acid molecule, encoding the gene of interest may be stably integrated into the genome of the cell, or the nucleic acid molecule may be present as an extrachromosomal molecule, such as a vector or plasmid. Such an extrachromosomal molecule may be auto-replicating. The term “transfection,” as used herein, refers to a process for introducing heterologous nucleic acid into a host cell.

The cells, undifferentiated or otherwise, may be used as dispersed cells or formed into clusters that may be infused into the hepatic portal vein. Alternatively, the cells may be provided in biocompatible degradable polymeric supports, porous non-degradable devices or encapsulated to protect from host immune response. The cells may be implanted into an appropriate site in a recipient. The implantation sites include, for example, the liver, natural pancreas, renal subcapsular space, omentum, peritoneum, subserosal space or a subcutaneous pocket.

To enhance further differentiation, survival or activity of implanted cells, additional factors, such as growth factors, antioxidants or anti-inflammatory agents, can be administered before, simultaneously with, or after the administration of the cells. In certain embodiments, growth factors may be utilized to differentiate the administered cells in vivo. These factors can be secreted by endogenous cells and exposed to the administered cells in situ. Implanted cells may be induced to differentiate by any combination of endogenous and exogenously administered growth factors known in the art.

The amount of cells used in implantation depends on a number of factors including the patient's condition and response to the therapy, and may be determined by one skilled in the art.

In one aspect, this invention provides a method for treating a patient suffering from, or at risk of developing diabetes. The method includes isolating and culturing cells, expanding the isolated population of cells, differentiating the cells into a β-cell lineage, or a pancreatic hormone-secreting cell in vitro, and incorporating the cells into a three-dimensional support. The cells can be maintained in vitro on this support prior to implantation into the patient. Alternatively, the support containing the cells can be directly implanted in the patient without additional in vitro culturing. The support can optionally be incorporated with at least one pharmaceutical agent that facilitates the survival and function of the transplanted cells.

Support materials suitable for use for purposes of the present invention include tissue templates, conduits, barriers, and reservoirs useful for tissue repair. In particular, synthetic and natural materials in the form of foams, sponges, gels, hydrogels, textiles, and nonwoven structures, which have been used in vitro and in vivo to reconstruct or regenerate biological tissue, as well as to deliver chemotactic agents for inducing tissue growth, are suitable for use in practicing the methods of the present invention. See, e.g., the materials disclosed in U.S. Pat. No. 5,770,417, U.S. Pat. No. 6,022,743, U.S. Pat. No. 5,567,612, U.S. Pat. No. 5,759,830, U.S. Pat No. 6,626,950, U.S. Pat. No. 6,534,084, U.S. Pat. No. 6,306,424, U.S. Pat. No. 6,365,149, U.S. Pat. No. 6,599,323, U.S. Pat. No. 6,656,488, and U.S. Pat. No. 6,333,029. Exemplary polymers suitable for use in the present invention are disclosed in US20040062753 and U.S. Pat. No. 4,557,264.

To form a support incorporated with a pharmaceutical agent, the pharmaceutical agent may be mixed with the polymer solution prior to forming the support. Alternatively, a pharmaceutical agent may be coated onto a fabricated support, preferably in the presence of a pharmaceutical carrier. The pharmaceutical agent may be present as a liquid, a finely divided solid, or any other appropriate physical form. Alternatively, excipients may be added to the support to alter the release rate of the pharmaceutical agent. In an alternate embodiment, the support is incorporated with at least one pharmaceutical compound that is an anti-inflammatory compound, such as, for example compounds disclosed in U.S. Pat. No. 6,509,369.

In one embodiment, the support is incorporated with at least one pharmaceutical compound that is an anti-apoptotic compound, such as, for example, compounds disclosed in U.S. Pat. No. 6,793,945.

In another embodiment, the support is incorporated with at least one pharmaceutical compound that is an inhibitor of fibrosis, such as, for example, compounds disclosed in U.S. Pat. No. 6,331,298.

In a further embodiment, the support is incorporated with at least one pharmaceutical compound that is capable of enhancing angiogenesis, such as, for example, compounds disclosed in US20040220393 and US20040209901.

In still another embodiment, the support is incorporated with at least one pharmaceutical compound that is an immunosuppressive compound, such as, for example, compounds disclosed in US20040171623.

In a further embodiment, the support is incorporated with at least one pharmaceutical compound that is a growth factor, such as, for example, members of the TGF-β family, including TGF-β1, 2, and 3, bone morphogenic proteins (BMP-2, -3, -4, -5, -6, -7, -11, -12, and -13), fibroblast growth factors-1 and -2, platelet-derived growth factor-AA, and -BB, platelet rich plasma, insulin growth factor (IGF-I, II) growth differentiation factor (GDF-5, -6, -8, -10, -15), vascular endothelial cell-derived growth factor (VEGF), pleiotrophin, endothelin, among others. Other pharmaceutical compounds can include, for example, nicotinamide, hypoxia inducible factor 1-alpha, glucagon like peptide-I (GLP-1), GLP-1 and GLP-2 mimetibody, and II, Exendin-4, nodal, noggin, NGF, retinoic acid, parathyroid hormone, tenascin-C, tropoelastin, thrombin-derived peptides, cathelicidins, defensins, laminin, biological peptides containing cell- and heparin-binding domains of adhesive extracellular matrix proteins such as fibronectin and vitronectin, MAPK inhibitors, such as, for example, compounds disclosed in US20040209901 and US20040132729.

The incorporation of the cells of the present invention into a scaffold may be achieved by the simple depositing of cells onto the scaffold. Cells may enter into the scaffold by simple diffusion (J. Pediatr. Surg. 23 (1 Pt 2): 3-9 (1988)). Several other approaches have been developed to enhance the efficiency of cell seeding. For example, spinner flasks have been used in seeding of chondrocytes onto polyglycolic acid scaffolds (Biotechnol. Prog. 14(2): 193-202 (1998)). Another approach for seeding cells is the use of centrifugation, which yields minimum stress to the seeded cells and enhances seeding efficiency. For example, Yang et al. developed a cell seeding method (J. Biomed. Mater. Res. 55(3): 379-86 (2001)), referred to as Centrifugational Cell Immobilization (CCI).

The present invention is further illustrated, but not limited by, the following examples.

### EXAMPLE 1

### Effects of Trichostatin A Treatment on Gene Expression in Panc-1 Cells and Neonatal Fibroblasts

Neonatal fibroblasts, also designated Hs27, were derived from human foreskins and obtained from the American Type Culture Collection (ATCC). Panc-1 cells are a transformed cell line derived from a pancreatic epitheloid carcinoma of ductal origin, also obtained from ATCC.

Fibroblasts or Panc-1 cells were seeded into a 6-well tissue culture plate at a density of 50,000 cells/cm². Both cell types were cultured in medium containing 10% FBS and DMEM under standard cell culture conditions (37° C., 5% CO₂). After reaching confluence (2-3 days), trichostatin A diluted in dimethyl sulfoxide (DMSO) and medium was added at either 2.5 μM or 5 μM to the cultures. Parallel cultures were treated with an equivalent concentration of DMSO as a vehicle control.

RNA samples were obtained from the treated cultures 48 hours after the addition of trichostatin A or DMSO. The culture medium was removed, the cells were washed with phosphate buffered saline (PBS), and RLT Lysis buffer containing β-mercaptoethanol (Qiagen) was added. The samples were homogenized using Qiashredder columns (Qiagen), and RNA was purified using the RNeasy Mini Kit (Qiagen). RNA quantity and quality was determined using a spectrophotometer, and cDNA was made using the iScript cDNA synthesis kit (BioRad).

The expression levels of Sox17, HNF-3 beta, Pdx-1, insulin, and glucagon were determined by Real-Time PCR (RT-PCR), as described in Example 15. Samples of 20 ng cDNA were used in each reaction. RT-PCR reactions were performed on the Applied Biosystems 7500, and data was analyzed using the accompanying software. Human pancreas cDNA was included as a positive control. Results were normalized against GAPDH expression levels.

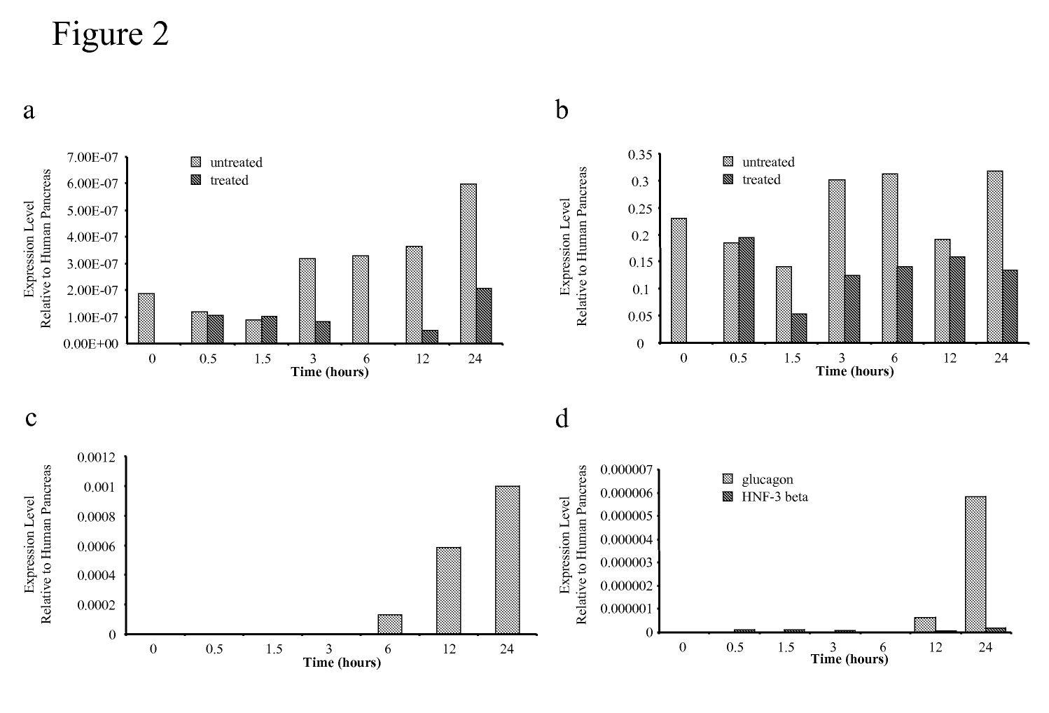

A basal level of expression for Sox17, HNF-3 beta, Pdx-1 and glucagon was detected in untreated Panc-1 cells. However, expression of these genes was not detectable in untreated neonatal fibroblasts. (Table I). Treatment of neonatal fibroblasts and Panc-1 cells with the HDAC inhibitor, trichostatin A, caused an increase in expression of Sox17, HNF-3 beta, Pdx-1, and glucagon (FIG. 2, panels a-d & Table I). Expression of insulin did not change relative to untreated controls in samples for either cell type under the conditions tested.

Basal expression levels of Sox17, HNF-3 beta, Pdx-1, and glucagon were higher in untreated Panc-1, compared to untreated neonatal fibroblast cells. Trichostatin A treatment evoked a more robust up-regulation of pancreatic gene expression in Panc-1 cells relative to fibroblasts, as measured for the representative endocrine and pancreas genes evaluated. Up-regulated expression of these genes also correlated in a dose-dependent manner with the concentration of trichostatin A used during treatment, again with a more robust effect noted in Panc-1 cells for these genes of interest. Panc-1 cells treated for 48 hours increased Sox-17 expression 60 times higher, Pdx-1 expression 11 times higher, and glucagon expression 5 times higher with 5.0 μm versus 2.5 μM trichostatin A. Similar dose response effects were noted for fibroblasts although the up-regulation was less pronounced overall (FIG. 2, panels a-d & Table I).

These data suggest that the potency of the HDAC inhibitor, trichostatin A, with respect to increasing expression of Sox17, HNF-3 beta, Pdx-1, and glucagon, is greater in Panc-1 cells that neonatal fibroblasts. This may also suggest that with a given treatment protocol, lineage specific gene expression can be enhanced to a greater extent in cells previously differentiated (or partially differentiated) along that same lineage pathway.

However, the effect of treatment is not restricted to differentiated (or partially differentiated) cells of that pathway but may encompass cells from other lineage pathways.

### EXAMPLE 2

### Effects of Trichostatin A on Gene Expression in Amniotic Fluid-Derived Cells

Amniotic fluid derived cells were seeded into 24-well tissue culture plates at a density of 5000/cm² and cultured in AMNIOMAX medium (Invitrogen) under standard cell culture conditions until confluent. Cells were obtained according to methods described in Example 14. After reaching confluence, 1.25 μM trichostatin A diluted in DMSO and medium was added to sample wells; an equivalent concentration of DMSO was added to control wells as a no treatment control.

RNA samples were obtained from treated cultures at 30 minutes, 1.5 hours, 3 hours, 6 hours, 12 hours, and 24 hours following addition of trichostatin A or DMSO. Culture medium was removed, cells were washed with PBS, and RLT lysis buffer with β-mercaptoethanol (Qiagen) was added. RNA was purified using the RNeasy Mini Kit (Qiagen). RNA quantity and quality was determined using a spectrophotometer, and cDNA was made using the iScript cDNA synthesis kit (BioRad).

Expression levels of Sox17, HNF-3 beta, Pdx-1, insulin, and glucagon were determined by Real-Time PCR. Samples of 20 ng cDNA were used in each reaction, performed on the Applied Biosystems 7500 according to methods described in Example 15. Data analysis was performed using the accompanying software. Human pancreas cDNA was included as a positive control, and results were normalized against GAPDH expression levels.

Basal expression of insulin and Sox-17 were consistently detected in untreated cells whereas low level expression of HNF-3 beta was detected intermittently at various time points in untreated cells. Glucagon and Pdx-1 gene expression were not detectable in untreated cells (Table II-A).

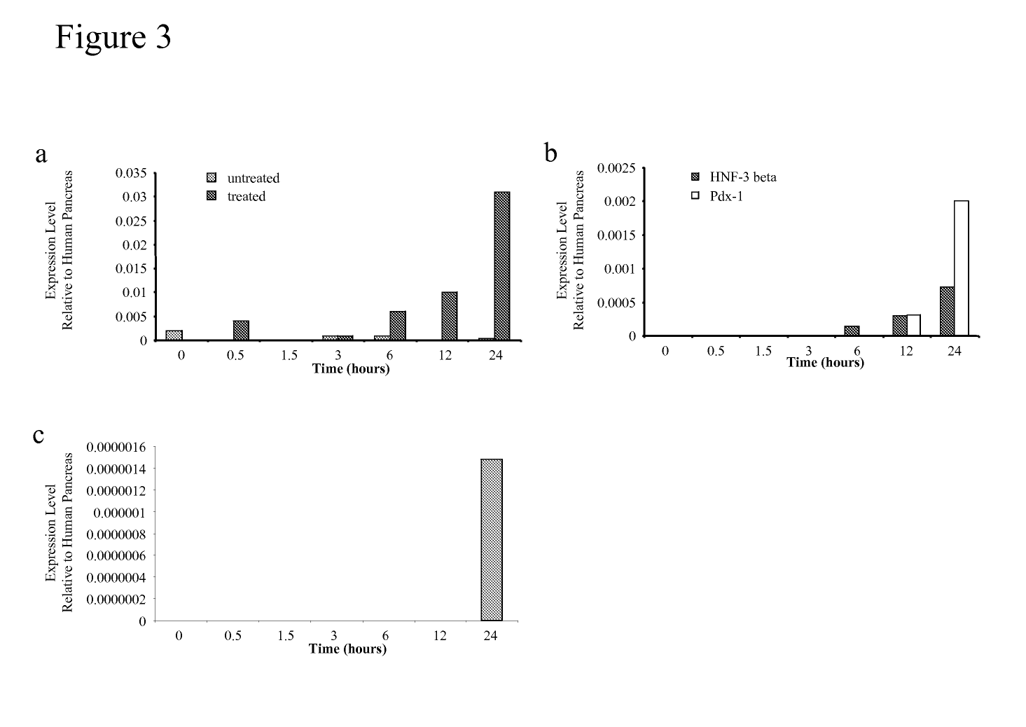

Treatment of amniotic fluid-derived cells with trichostatin A caused a decrease in the gene expression of insulin, had relatively little or no effect on Sox-17 expression, but induced an increase in gene expression of glucagon, HNF-3 beta and Pdx-1 over time (FIG. 3, panels a-d & Table II-B). HNF-3 beta gene expression in amniotic fluid-derived cells was detectable by RT-PCR at >35 cycles by 30 minutes after addition of trichostatin A, increasing with time to <35 cycles at 24 hours. Pdx-1 gene expression was undetectable 30 minutes after addition of trichostatin A, first detectable at >35 cycles by RT-PCR at 6 hours, increasing to <35 cycles or ˜0.1% of human pancreas levels at 24 hours. Glucagon expression was undetectable 30 minutes after addition of trichostatin A, first detectable at >35 cycles by RT-PCR at 12 hours, increasing to <24 cycles at 24 hours (FIG. 3, panels c-d).

These results, in conjunction with results from Panc-1 cells in Example 1, suggest a pattern in regulation of gene sets inherent in a particular cell lineage differentiation program. Differentiated cells treated with a chromatin-remodeling agent may down-regulate some genes and up-regulate other genes in response to both the presence of the HDAC inhibitor and environmental or other stimulatory signals.

### EXAMPLE 3

### Effects of Trichostatin A on Gene Expression in Late Passage (P14) Pancreatic-Derived Stromal Cells

Human pancreatic-derived stromal cells were obtained according to the methods described in Example 13. Cells were seeded into a 24-well tissue culture plate at a density of 5000/cm² and cultured in DMEM containing 10% fetal bovine serum under standard cell culture conditions until confluent. After the cells reached confluency, 2.5 μM trichostatin A, diluted in DMSO and medium was added to the wells. Parallel cultures were treated with an equivalent concentration of DMSO as a vehicle control. RNA samples were obtained from treated cultures at 30 minutes, 1.5 hours, 3 hours, 6 hours, 12 hours and 24 hours following the addition of trichostatin A or DMSO. Culture medium was removed, the cells were washed with PBS, and RLT lysis buffer with β-mercaptoethanol (Qiagen) was added. RNA was purified using the RNeasy Mini Kit (Qiagen). RNA quantity and quality were determined using a spectrophotometer, and CDNA was made using the iScript cDNA synthesis kit (BioRad).

The expression levels of Sox17, HNF-3 beta, Pdx-1, insulin, and glucagon were determined by Real-Time PCR. Samples of 20 ng cDNA were used in each reaction, which was performed on the Applied Biosystems 7500 according to the methods described in Example 15. Data were analyzed using the accompanying software. Human pancreas cDNA was included as a positive control, and results were normalized against GAPDH expression levels.

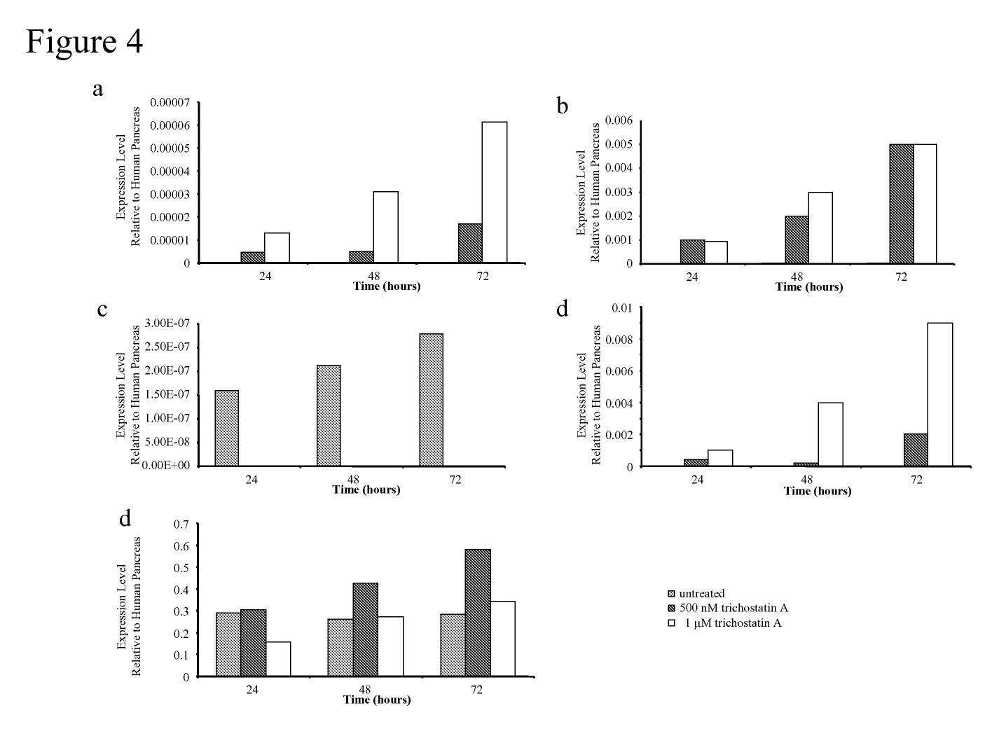

Expression of insulin, Pdx-1, glucagon or HNF-3 beta genes was not detected in untreated late passage pancreatic-derived stromal cells. However, Sox-17, was detected at low levels intermittently in untreated cells at various time points during the course of the experiment (Table III-A). Treatment of late passage pancreatic-derived stromal cells with 2.5 μM of trichostatin A caused an increase in gene expression of Sox-17, glucagon, HNF-3 beta, and Pdx-1 genes over time. However, no changes in insulin gene expression were observed (FIG. 4, panels a-c & Table III-B).

Sox-17 gene expression in late passage pancreatic-derived stromal cells was consistently detectable at >35 cycles by RT-PCR 3 hours after addition trichostatin A, increasing over time to levels <35 cycles or ˜3% of human pancreas levels at 24 hours (FIG. 4, panel a). HNF-3 beta gene expression was detectable at >35 cycles by RT-PCR by 6 hours after addition of trichostatin A, increasing to <35 cycles or ˜0.075% of human pancreas at 24 hours (FIG. 4, panel b). Pdx-1 gene expression was detectable at >35 cycles by RT-PCR by 12 hours after addition of trichostatin A, increasing to <35 cycles or ˜0.2% of human pancreas levels at 24 hours (FIG. 4, panel b). Glucagon expression was undetectable until 24 hours following addition of trichostatin A, reaching detectable levels of 35-40 cycles by RT-PCR at that time point (FIG. 4, panel c). Collectively these data indicate that treatment with the HDAC inhibitor agent trichostatin A can induce or enhance expression of endocrine pancreatic genes in cells that have low basal expression rates or that have lost previous expression patterns over time in culture.

### EXAMPLE 4

### Effects of Chronic Trichostatin A Treatment on Gene Expression in Amniotic Fluid-Derived Cells

Amniotic fluid derived cells were obtained according to the methods described in Example 14. Cells were seeded into a 24-well tissue culture plate at a density of 5000/cm² and cultured in AMNIOMAX (Invitrogen) under standard cell culture conditions until confluent. After the cells reached confluency, sample wells were treated with either 500 nM or 1.0 μM trichostatin A diluted in DMSO and medium; control wells were treated with an equivalent concentration of DMSO. At 24 hour intervals over the three day incubation period, cultures underwent a complete medium change, and a fresh dilution of trichostatin A or DMSO was added as appropriate.

RNA samples were obtained from the treated cultures at 24 hour intervals after the initial addition of trichostatin A or DMSO. The culture medium was removed, cells were washed with PBS, and RLT lysis buffer with P-mercaptoethanol (Qiagen) was added. RNA was purified using the RNeasy Mini Kit (Qiagen); RNA quantity and quality was determined using a spectrophotometer. cDNA was made using the iScript cDNA synthesis kit (BioRad).

Expression levels of Sox17, HNF-3 beta, Pdx-1, insulin, and glucagon were determined by RT-PCR. Samples of 20 ng cDNA were used in each reaction, performed on an Applied Biosystems 7500 according to the methods described in Example 15. Data were analyzed using the accompanying software. Human pancreas cDNA was included as a control. Results were normalized against GAPDH expression levels. Basal expression of insulin and Sox-17 genes was detectable in untreated amniotic fluid-derived cells. However, expression of glucagon and Pdx-1 genes was not detectable in untreated cells by RT-PCR up to cycle 40. HNF-3 beta was not detected in untreated cells at 24-hours in culture but was weakly expressed at 48 and 72 hours culture time.

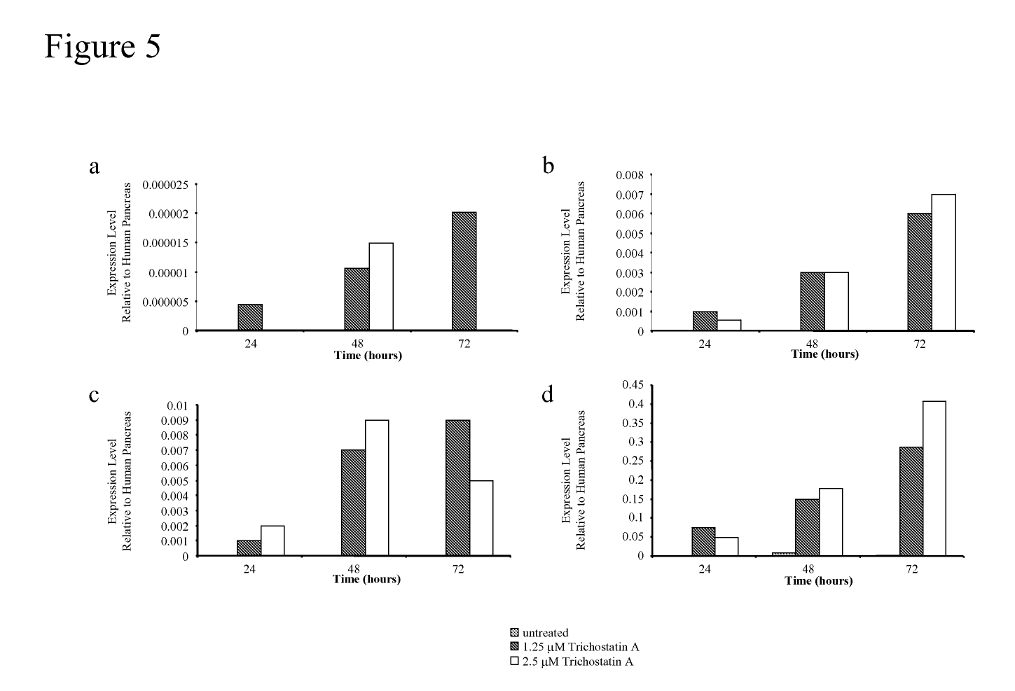

Following treatment with trichostatin A, amniotic fluid-derived cells expressed glucagon, HNF-3 beta, and Pdx-1 above basal levels (FIG. 5, panels a-e, Table IV). Gene expression for glucagon and Pdx-1 increased in a dose and time dependent manner after treatment with trichostatin A, with highest expression seen at the final 72 hour time point and with the higher 1 μM treatment dose (FIG. 5, panels a & d). Similarly, HNF-3 beta gene expression in this example also increased in a time dependent manner with highest expression observed at the final 72 hour time point. However, HNF-3 beta expression was essentially equivalent with both treatment doses of 1 μM and 500 nM trichostatin A (FIG. 5, panel b). In contrast, insulin gene expression decreased after treatment to non-detectable levels at all time points. Sox-17 expression was affected to a less significant degree relative to initial basal expression levels with both treatment doses of trichostatin A. This suggests that the extent of the induced gene expression in response to treatment with an HDAC inhibitor may be variable.

### EXAMPLE 5

### Effects of Chronic Trichostatin A Treatment on Gene Expression in Late Passage Pancreatic-Derived Stromal Cells

Pancreatic-derived stromal cells were obtained according to the methods described in Example 13. Cells were seeded into a 24-well tissue culture plate at a density of 5000/cm² and cultured in DMEM with 10% FBS under standard cell culture conditions until confluent. After the cells reached confluency, sample wells were treated with either 1.25 μM or 2.5 μM trichostatin A diluted in DMSO and medium; control wells received DMSO at an equivalent concentration. At 24 hour intervals over the three day culture period, cultures underwent a complete medium change, and a fresh dilution of trichostatin A or DMSO was added as appropriate. RNA samples were obtained from treated cultures daily after the initial addition of trichostatin A or DMSO. Culture medium was removed, cells were washed with PBS, and RLT lysis buffer with β-mercaptoethanol (Qiagen) was added. RNA was purified using the RNeasy Mini Kit (Qiagen), and RNA quantity and quality was determined using a spectrophotometer. cDNA was made using the iScript cDNA synthesis kit (BioRad).

Expression levels of Sox17, HNF-3 beta, Pdx-1, insulin, and glucagon were determined by RT-PCR. Samples of 20 ng cDNA were used in each reaction, which was performed on the Applied Biosystems 7500, according to the methods described in Example 15. Data were analyzed using the accompanying software. Human pancreas cDNA was included as a control. Results were normalized against GAPDH expression levels.

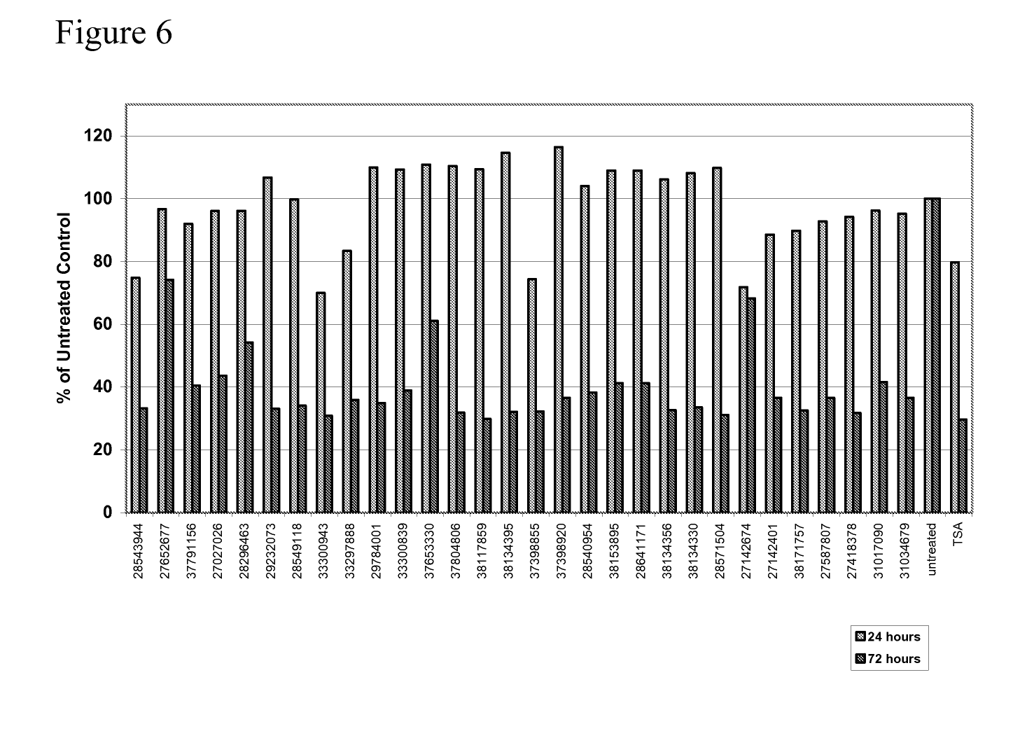

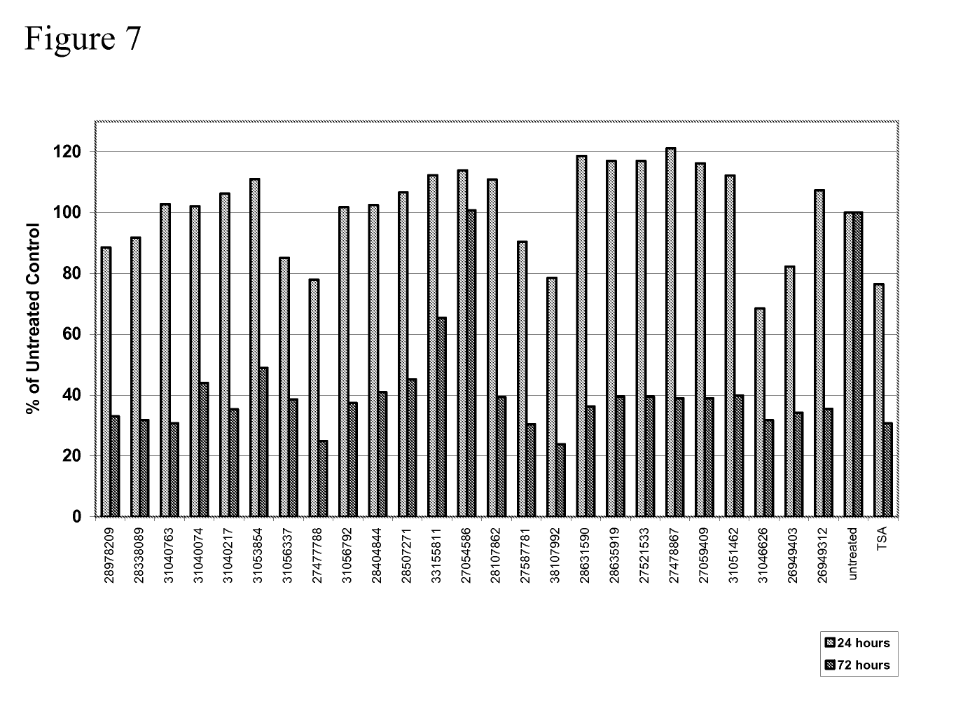

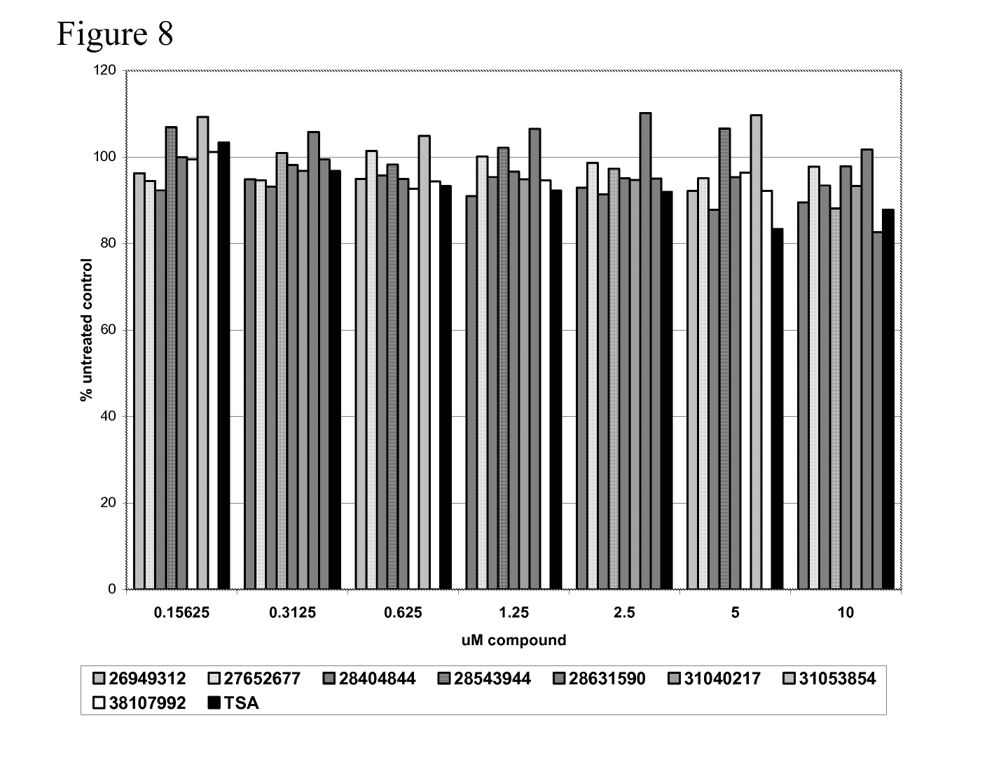

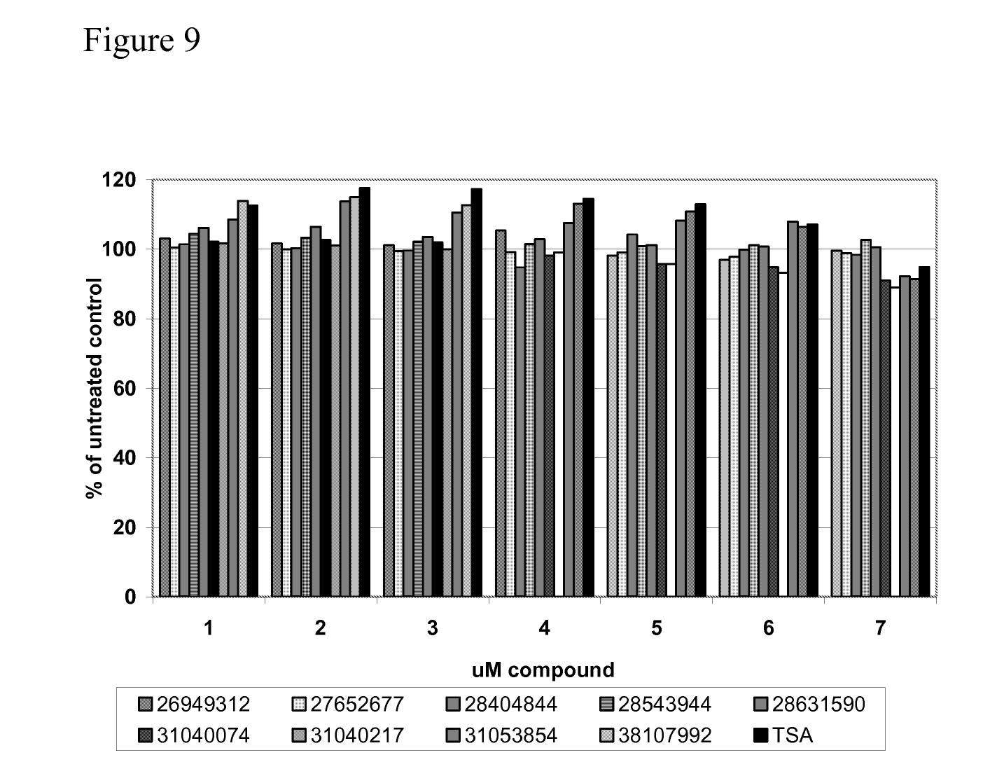

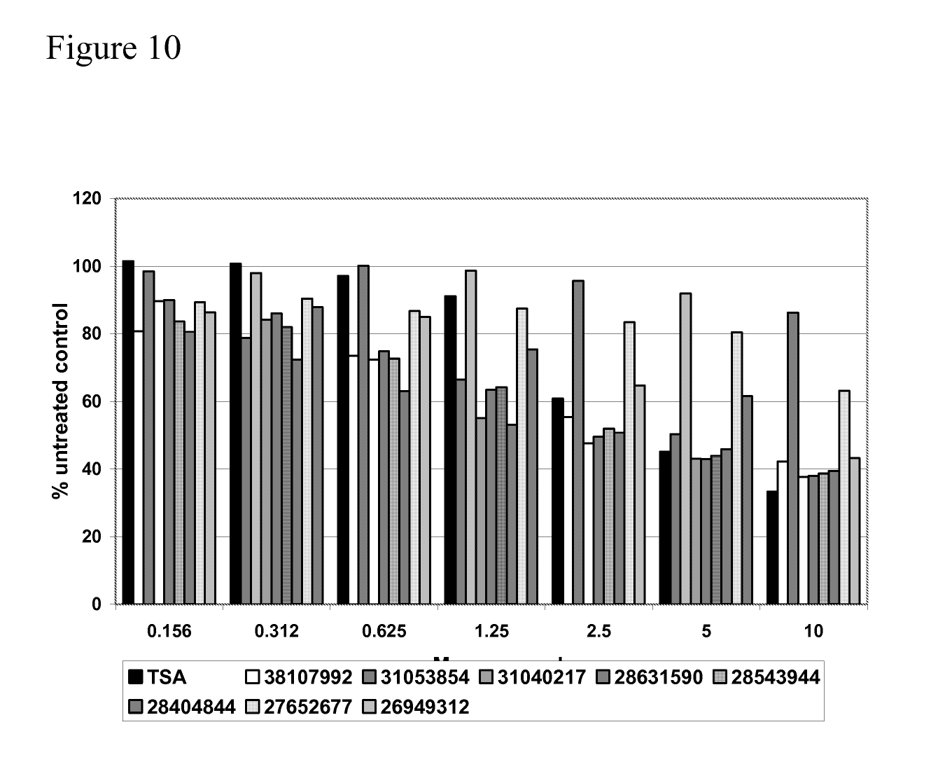

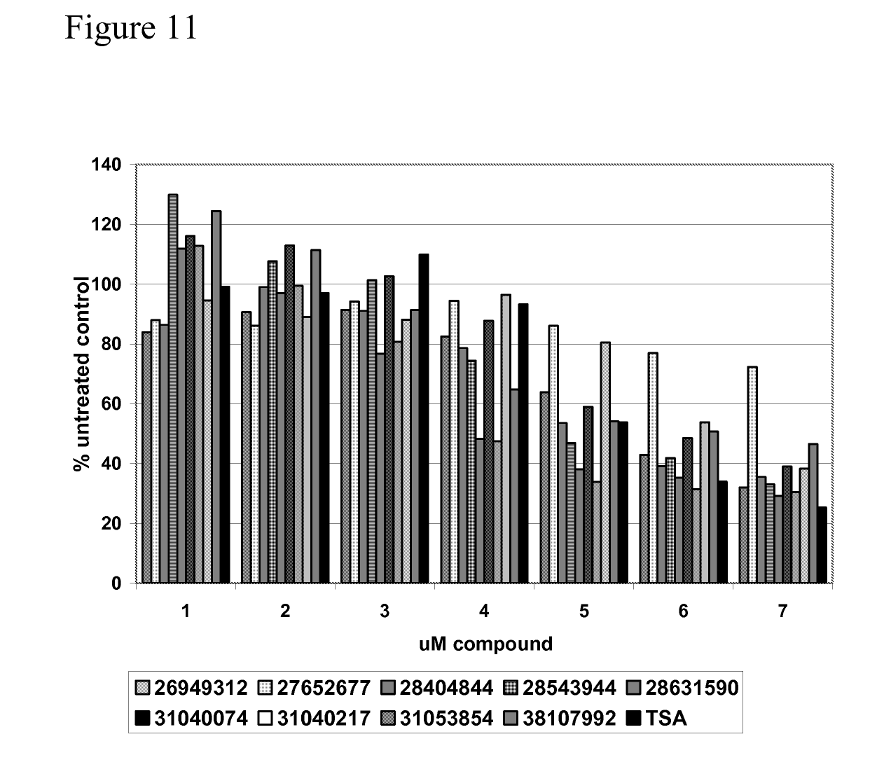

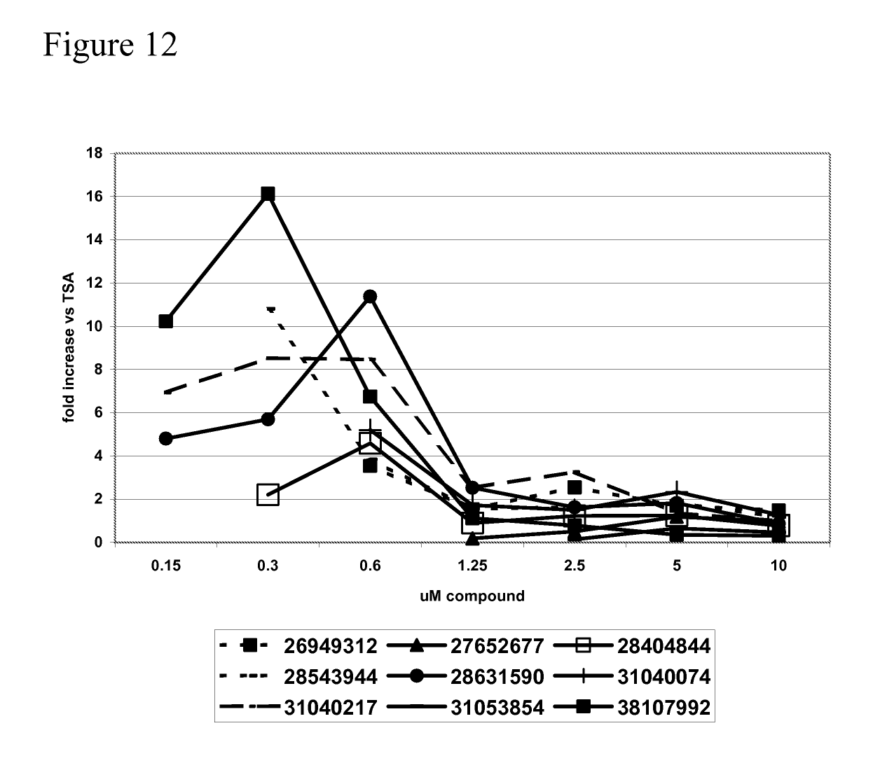

Glucagon, HNF-3 beta, Pdx-1 and Sox-17 gene expression was not detectable by RT-PCR in untreated late passage pancreatic-derived stromal cells. After addition of trichostatin A, gene expression was detectable for all of the aforementioned genes. Insulin gene expression was undetectable prior to treatment and did not increase following trichostatin A treatment. Glucagon, HNF-3 beta, Pdx-1, and Sox-17 gene expression levels all increased in a time dependent manner with highest expression observed for all three genes at 72 hours. In some cases differences in expression levels could be seen when a higher concentration of trichostatin A was added to the cells (FIG. 6, panels a-d & Table V). Increases in gene expression were equivalent with these two treatment concentrations of 1.25 and 2.5 μM trichostatin A; differences at each time point for each gene were minimal or did not appear to be significant. The data imply that for a given gene, there may be a maximal threshold level for treatment by an HDAC inhibitor to have an effect on gene expression at a given time point and that increasing the concentration of HDAC inhibitor has no added benefit. The data may also suggest that continuous replenished addition of trichostatin A during the treatment protocol may be necessary to sustain escalating increases in gene expression over time.

### EXAMPLE 6

### Effects of Chronic Trichostatin A Treatment on Gene Expression in Amniotic Fluid-Cells and Pancreatic-Derived Stromal Cells

Several cell lines obtained from different amniotic fluid specimens (see Example 14) and pancreas donors (see Example 13) were tested with similar results. Two lines at similar passage number but derived from different amniotic fluid specimens are shown in Table VI-A and Table VI-B for this example. One of these cell lines was also used in examples 2 and 4 above. In addition, this example also contains comparison data in Table VI-C and Table VI-D for a single pancreatic-derived stromal line grown to early and late passage number.

Amniotic fluid derived cells or pancreatic-derived stromal cells were seeded into 24-well tissue culture plates at a density of 5000/cm² and cultured in AMNIOMAX (Invitrogen) or DMEM with 10% FBS, respectively, under standard cell culture conditions until confluent. After the cells reached confluency, amniotic fluid derived cells were treated once at time 0 hours with 500 nM trichostatin A. Pancreas-derived stromal cells were treated once at time 0 hours with 1.25 μM trichostatin A. Samples for RT-PCR were taken daily from zero to six days at the times indicated in Table VI.

RNA samples were obtained from the treated cultures up to 144 hours after the initial addition of trichostatin A or DMSO. Culture media was removed, the cells were washed with PBS, and RLT lysis buffer containing β-mercaptoethanol (Qiagen) was added. During culture, the medium was not changed nor was freshly prepared trichostatin A added. RNA was purified using the RNeasy Mini Kit (Qiagen), and RNA quantity and quality was determined using a spectrophotometer. cDNA was made using the iScript cDNA synthesis kit (BioRad).

Samples of 20 ng cDNA were used in each reaction to determine the expression levels of the following genes in amniotic fluid-derived cells: Gata1, HNF-3 beta, Pdx-1, insulin, and Sox17. Similarly, samples of 20 ng cDNA were used in each reaction to determine the expression level of the following genes in pancreatic-derived stromal cells: glucagon, HNF-3 beta, insulin and Pdx-1. Real-Time PCR was performed on the Applied Biosystems 7500, and data was analyzed using the accompanying software according to the methods described in Example 15.

Analysis of samples obtained from the untreated amniotic fluid-derived cell line used in Examples 2 & 4 above showed that these cells did not express HNF-3 beta or Gata1 but did express Sox-17 and very low levels of insulin and Pdx-1 (Table VI-A). After addition of trichostatin A, HNF-3 beta expression increased, but only during the first 48 hours after initial treatment. Pdx-1 was expressed continuously for 24 hours following treatment, but expression was not detectable after that time point. The insulin gene was not expressed for 48 hours following initial treatment but was detectable after 48 hours. There was no change in Sox17 or Gata1 expression observed (Table VI-A). These data suggest that trichostatin A may inhibit insulin gene expression in these cells within the initial 24-48 hours of culture but is associated with up regulation of Pdx-1 and HNF-3 beta expression within the same time period, followed by a return to undetectable or very weak expression after 48 hours. Trichostatin A may degrade in culture and may not have a significant sustained impact on gene expression at later time points after 24-48 hours. Consequently, the net effect over long time periods using a single initial dose of trichostatin A may reflect the reversible nature of this reagent.

Analysis of samples obtained from an additional untreated amniotic fluid-derived cell line showed that these cells did not express basal levels of HNF-3 beta, Pdx-1 or Gata-1 but did express insulin and Sox-17. Following trichostatin A treatment, HNF-3 beta expression was detected at 48 hours, but as seen in the first amniotic fluid derived cell line, HNF-3 beta decreased to very weak expression levels after 48 hours (Table VI-B). Pdx-1 gene expression was only detected at 24 hours following trichostatin A treatment and returned to undetectable levels for the remainder of the experiment. Insulin gene expression fell to undetectable levels following the addition of 500 nM trichostatin A and then returned to detectable levels starting at 72 hours after the addition of trichostatin A (Table VI-B). Expression of Sox17 and Gata1 genes did not change with the addition of trichostatin A. These data suggest that trichostatin A may be metabolized within 24-48 hours of addition to cell culture samples and that trichostatin A inhibits insulin gene expression in these cells but has a reversible stimulatory effect on other genes including HNF-3 beta and Pdx-1 (Table VI-B).

Prior to treatment, early passage (P5) pancreatic-derived stromal cells expressed Pdx-1, while expression of the glucagon, HNF-3 beta and insulin genes was not detectable. At 24 hours following treatment with 2.5 μM trichostatin A, HNF-3 beta, Pdx-1, and glucagon genes all demonstrated up-regulated expression, but the effect was not sustained and only glucagon expression was still detectable 48 hours after initial treatment, albeit decreased to a lower level than observed at 24 hours. Insulin gene expression remained undetectable and unchanged throughout the course of the experiment (Table VI-C). These data suggest that following trichostatin A treatment, expression of some genes, e.g. transcription factors, may be short-lived depending on the inherent instability of the corresponding mRNA, and/or secondary effects of an HDAC inhibitor on mRNA stability, and/or additional negative regulatory pathways operating in these cells. More persistent expression of other genes over a similar time period may reflect greater message stability and/or additional positive regulatory effects.

Untreated late passage (P14) pancreatic-derived stromal cells did not express any of the genes of interest, suggesting that the expression of these endocrine pancreatic markers declines with time during in vitro culture maintenance. Following treatment with trichostatin A, insulin gene expression remained undetectable and unchanged throughout the course of the experiment (Table VI-D). Glucagon, Pdx-1, and HNF-3 beta gene expression were detected by RT-PCR at 24 hours (Table VI-D). However, only glucagon expression remained detectable 48 hours after initial treatment albeit at a lower expression level than observed at 24 hours and declined to undetectable levels thereafter. The glucagon gene was expressed for a shorter period of time in these late passage cells as compared to early passage pancreatic-derived stromal cells which may reflect a less plastic stage of response to the effects of HDAC inhibitors.

### EXAMPLE 7

### Effects of Two Sequential Low Dose Trichostatin A Treatments on Gene Expression in Amniotic Fluid-Cells and Pancreatic-Derived Stromal Cells

Several cell lines obtained from different amniotic fluid specimens (see Example 14) and pancreas donors (see Example 13) were tested with similar results. Two lines at similar passage number but derived from different amniotic fluid specimens are shown in Table VII-A and Table VII-B for this example. One of these cell lines was also used in examples 2 and 4 above. In addition, this example also contains comparison data in Table VII-C and Table VII-D for a single pancreatic-derived stromal line grown to early and late passage number.

Amniotic fluid derived cells or pancreatic-derived stromal cells were seeded into 24-well tissue culture plates at a density of 5000/cm² and cultured in AMNIOMAX (Invitrogen) or DMEM with 10% FBS respectively under standard cell culture conditions until confluent. After the cells reached confluency, amniotic fluid derived cells were treated at time 0 hours with a dose of 500 nM trichostatin A, and pancreatic-derived stromal cells were treated with a dose of 1.25 μM trichostatin A solubilized in DMSO and medium. At time 24 hours, cell cultures underwent a complete medium change to remove all traces of trichostatin A. At time 96 hours, cell cultures were re-fed with fresh medium and received a second dose of trichostatin A at the same concentration as used previously. At time 120 hours, cell cultures again underwent a complete medium change and were cultured for an additional 24 hours or 148 hours total incubation time.

Samples were taken daily for RT-PCR at the times indicated in Table VII-A to D. The culture medium was removed, and samples were rinsed in PBS then collected in RLT with β-mercaptoethanol (Qiagen). RNA was purified using the RNeasy Mini Kit (Qiagen) and RNA quantity and quality were determined using a spectrophotometer. cDNA was made using the iScript cDNA synthesis kit (BioRad).

Samples of 20 ng cDNA were used in each reaction to determine the expression levels of the following genes in amniotic fluid-derived cells: Gata1, HNF-3 beta, Pdx-1, insulin, and Sox17. Similarly, samples of 20 ng cDNA were used in each reaction to determine the expression level of the following genes in pancreatic-derived cells: glucagon, HNF-3 beta, insulin and Pdx-1. Real-Time PCR was performed on the Applied Biosystems 7500, and data was analyzed using the accompanying software according to the methods described in Example 15.

Prior to treatment with trichostatin A, the amniotic fluid-derived cell line used in Examples 2 & 4 did not express Gata-1 or HNF-3 beta. However, expression of the insulin, Pdx-1, and Sox-17 genes was detected by RT-PCR at 30, 37 cycles and 27 cycles respectively(Table VII-A). Following treatment with 0.5 μM trichostatin A, Sox-17 and Gata-1 gene expression levels did not change at any subsequent time point throughout the experiment. Insulin expression was detected by RT-PCR at 36 cycles prior to trichostatin A treatment but decreased to undetectable levels at 24 hours following the first addition of trichostatin A and reappeared at detectable levels of 36 cycles by 48 hours. Gene expression remained detectable at 36 cycles for the duration of the experiment. HNF-3 beta was detected by RT-PCR at 33 cycles following the first trichostatin A treatment, decreasing to undetectable levels following the first medium change, reappearing at 37 cycles following the second treatment with trichostatin A, and remaining for the duration of the experiment. Pdx-1 gene expression was detected at 37 cycles by RT-PCR, continuing at this level for 24 hours following the addition of trichostatin A, but was not detectable after that time point for the remainder of the experiment (Table VII-A).

Prior to treatment with trichostatin A, the amniotic fluid-derived cell line used in Example 6 did not express Gata-1, HNF-3 beta or Pdx-1. Expression of insulin and Sox-17 was detected by RT-PCR at cycle 35 and cycle 27 respectively (Table VII-B). Following treatment with 0.5 μM trichostatin A, HNF-3 beta was expressed at 34 cycles by RT-PRC until 72 hours; thereafter the expression level declined for the remainder of the experiment to cycle 37 as detected by RT-PCR. Pdx-1 gene expression was detected by 24 hours at cycle 39 by RT-PCR following trichostatin A treatment, but thereafter declined and was undetectable for the remainder of the experiment. Insulin gene expression was not detectable for the first 48 hours of the experiment following trichostatin A treatment but was detected by RT-PCR at cycle 34 at 72 hours and for the remainder of the experiment. Sox-17 and Gata-1 gene expression levels did not change throughout the course of the experiment (Table VII-B).

Early passage (P5) pancreatic-derived stromal cells did not express glucagon, HNF-3 beta or insulin prior to treatment with trichostatin A. However, Pdx-1 expression was detected by RT-PCR at cycle 38. Following treatment with 1.25 μM trichostatin A, there was no change in insulin expression. Pdx-1 and HNF-3 beta gene expression were detectable by RT-PCR at cycle 34 for both genes 24 hours following the addition of trichostatin A; thereafter expression fell to undetectable levels until after the second addition of trichostatin A, after which detection was at cycle 33 for Pdx-1 and HNF-3 beta. Glucagon gene expression was detected by RT-PCR at cycle 34 for the first 48 hours after addition of trichostatin A but fell to undetectable levels until after the second treatment cycle of trichostatin A, at which time the expression level increased to cycle 37 by RT-PCR (Table VII-C).

Similar results were seen for late passage (P11) pancreatic-derived stromal cells: Glucagon, HNF-3 beta, insulin, and Pdx-1 were undetectable prior to treatment. Following treatment with 1.25 μM trichostatin A there was no change in insulin gene expression; however, expression of glucagon, HNF-3β and Pdx-1 genes could be detected by RT-PCR at cycle 33 for all three genes. Gene expression decreased thereafter or was undetectable until after the second treatment cycle of trichostatin A, where expression levels were detected by RT-PCR to be at cycle 36 for glucagon gene and 34 for HNF-3 beta and Pdx-1 genes (Table VII-D).

Collectively these data imply that effects of trichostatin A on gene expression are not sustained after its removal from culture. However, repeat applications of trichostatin A can restore it's effects on gene expression. The overall pattern of gene expression evoked by trichostatin A may depend in part on the dose concentration used, the duration of treatment, and the interval between treatment periods.

### EXAMPLE 8

### Effects of Two Sequential High Dose Trichostatin A Treatments on Gene Expression in Amniotic Fluid-Cells and Pancreatic-Derived Stromal Cells

Several cell lines obtained from different amniotic fluid specimens (see Example 14) and pancreas donors (see Example 13) were tested with similar results. Two lines at similar passage number but derived from different amniotic fluid specimens are shown in Table VIII-A and Table VIII-B for this example. One of these cell lines was also used in examples 2 and 4 above. In addition, this example also contains comparison data in Table VIII-C and Table VIII-D for a single pancreatic-derived stromal line grown to early and late passage number.

Amniotic fluid derived cells or pancreatic-derived stromal cells were seeded into 24-well tissue culture plates at a density of 5000/cm² and cultured in AMNIOMAX (Invitrogen) or DMEM with 10% FBS respectively under standard cell culture conditions until confluent. After the cells reached confluency, amniotic fluid derived cells were treated at time 0 hours with a dose of 1.25 μM trichostatin A, and pancreatic-derived stromal cells were treated with a dose of 5.0 μM trichostatin A solubilized in DMSO and medium. At time 24 hours, cell cultures underwent a complete medium change to remove all traces of trichostatin A. At time 96 hours, cell cultures were re-fed with fresh medium and received a second dose of trichostatin A at the same concentration as used previously. At time 120 hours, cell cultures again underwent a complete medium change and were cultured for an additional 24 hours or 148 hours total incubation time.

Samples were taken daily for RT-PCR at the times indicated in Table VIII-A to D. The culture media was removed, and samples were rinsed in PBS then collected in RLT with β-mercaptoethanol (Qiagen). RNA was purified using the RNeasy Mini Kit (Qiagen) and RNA quantity and quality were determined using a spectrophotometer. cDNA was made using the iScript cDNA synthesis kit (BioRad).

Samples of 20 ng cDNA were used in each reaction to determine the expression levels of the following genes in amniotic fluid-derived cells: Gata1, HNF-3 beta, Pdx-1, insulin, and Sox17. Similarly, samples of 20 ng cDNA were used in each reaction to determine the expression level of the following genes in pancreatic-derived cells: glucagon, HNF-3 beta, insulin and Pdx-1. Real-Time PCR was performed on the Applied Biosystems 7500, and data was analyzed using the accompanying software according to the methods described in Example 15.

Prior to treatment with trichostatin A, the amniotic fluid-derived cell line used in Examples 2 & 4 did not express Gata-1 or HNF-3 beta. Gene expression for insulin, Pdx-1, and Sox-17 was detected by RT-PCR at 36 cycles, 37 cycles, and 27 cycles, respectively. Treatment with 1.25 μM trichostatin A caused HNF-3 beta expression which was detected at 32 cycles; however, this expression was transient as HNF-3 beta gene expression levels declined following the initial treatment with 1.25 μM trichostatin A to undetectable levels. A second treatment with 1.25 μM trichostatin A caused HNF-3 beta gene expression to be detected at 32 cycles as measured by RT-PCR (Table VIII-A). Similar results were observed for Pdx-1 gene expression. Treatment with 1.25 μM trichostatin A caused an increase in gene expression of Pdx-1 as measured by a decrease in cycles from 37 to 32, as measured by RT-PCR. This increase was transient, however, as Pdx-1 gene expression levels declined to undetectable levels following the initial treatment with 1.25 μM trichostatin A. A second treatment with 1.25 μM trichostatin A caused Pdx-1 gene expression to be detected at 34 cycles as measured by RT-PCR (Table VIII-A). Gata-1 expression was only detected at 37 cycles by RT-PCR following the first addition of trichostatin A. Sox-17 gene expression did not change with the addition of trichostatin A. Insulin gene expression was undetectable following 1.25 μM trichostatin A treatment but was detected at 36 cycles as measured by RT-PCR following the media change (Table VIII-A).

Prior to treatment with trichostatin A, the amniotic fluid-derived cell line used in Example 6 did not express Gata-1, HNF-3 beta and Pdx-1. However, expression of insulin and Sox-17 was detected by RT-PCR at 35 cycles and 27 cycles respectively. The initial 1.25 μM trichostatin A treatment stimulated expression of HNF-3 beta and Pdx-1. HNF-3 beta expression was detected at 35 cycles as measured by RT-PCR but that level of expression decreased to 38 cycles prior to the second addition of trichostatin A. Following the second 1.25 μM trichostatin A treatment HNF-3 beta gene expression returned to detection at 32 cycles as measured by RT-PCR. Pdx-1 expression was detected by RT-PCR at 33 cycles after the initial 1.25 μM trichostatin A treatment. This level of expression was not detectable after the trichostatin A was removed from the medium but returned to detectable levels at cycle 32 as measured by RT-PCR, following the second 1.25 μM trichostatin A treatment (Table VIII-B). Gata-1 gene expression did not change throughout the course of the experiment. While insulin gene expression was detected prior to trichostatin A treatment at 35 cycles as measured by RT-PCR, it was not detectable immediately following treatment. After trichostatin A was washed away, detection of insulin gene expression returned and was detectable at 36 cycles as measured by RT-PCR. Following the second addition of trichostatin A, insulin gene expression was once again undetectable. Sox-17 gene expression was unchanged for the duration of the experiment (Table VIII-B). These data suggest that 1.25 μM trichostatin A is a better concentration to use than 0.5 μM trichostatin A for increasing HNF-3β and Pdx-1 gene expression in this cell. The effects of 1.25 μM trichostatin A seem have a prolonged effect after a change of medium, indicating that the effect on gene expression persists beyond the actual time the compound is present. It also appears that trichostatin A may have an inhibitory effect on insulin gene expression.

Expression of glucagon, HNF-3 beta and insulin genes was not detected in early passage (P5) pancreatic-derived stromal cells prior to treatment with 5.0 μM trichostatin A although Pdx-1 expression was observed at 38 cycles as measured by RT-PCR (Table VIII-C). Following treatment with 5.0 μM trichostatin A, HNF-3 beta and glucagon expression was detected at 34 cycles and 35 cycles respectively as measured by RT-PCR. HNF-3 beta gene expression was not detectable after trichostatin A was removed, but gene expression was restored and detectable at cycle 36 by RT-PCR following a second treatment with trichostatin A. The level of expression of glucagon also decreased when trichostatin A was removed and was not detectable following the second addition of TSA until the end of the experiment when it was detected at 38 cycles by RT-PCR. Pdx-1 gene expression was detected only after the second treatment of trichostatin A at cycle 32 as measured by RT-PCR. Insulin gene expression did not change throughout the course of the experiment (Table VIII-C). These data suggest that 24 hours of treatment with 5.0 μM trichostatin A was sufficient to increase gene expression of HNF-3 beta and Pdx-1 but was not sufficient to increase insulin gene expression in early passage (P5) pancreatic-derived stromal cells.

Results for late passage (P1) pancreatic-derived stromal cells were similar to those seen for early passage (P5) pancreatic-derived stromal cells. Prior to trichostatin A treatment, no genes of interest were detectable, but following treatment with 5.0 μM trichostatin A, glucagon was detected at cycle 33, HNF-3 beta was detected at cycle 32 and Pdx-1 expression was detected at cycle 31 as measured by RT-PCR. Following the medium change, glucagon expression was still detectable by RT-PCR but the levels observed decreased to 38 cycles as measured by RT-PCR and did not increase with the second addition of trichostatin A. HNF-3 beta gene expression was detectable following the first trichostatin A treatment at 32 cycles but was undetectable following the change of medium; expression was not detectable again until after the second treatment of trichostatin A at 36 cycles as measured by RT-PCR. Pdx-1 was expressed at 31 cycles following the initial treatment with trichostatin A, but this level of expression was not detectable after the trichostatin A was removed. Pdx-1 gene expression was detected by RT-PCR at 34 cycles following the second addition of trichostatin A. There was no change in insulin gene expression following trichostatin A treatment or withdrawal (Table VIII-D). These data suggest that 24 hours of treatment with 5.0 μM trichostatin A was not sufficient to increase insulin gene expression in late passage (P1) pancreatic-derived stromal cells, but that 5.0 μM trichostatin A was sufficient to increase gene expression of HNF-3 beta and Pdx-1.

### EXAMPLE 9

### Effects of Two High Dose Trichostatin A Treatments on Gene Expression in Amniotic Fluid-Cells and Pancreatic-Derived Stromal Cells

Several cell lines obtained from different amniotic fluid specimens (see Example 14) and pancreas donors (see Example 13) were tested with similar results. Two lines at similar passage number but derived from different amniotic fluid specimens are shown in Table VIII-A and Table VIII-B for this example. One of these cell lines was also used in examples 2 and 4 above. In addition, this example also contains comparison data in Table VIII-C and Table VIII-D for a single pancreatic-derived stromal line grown to early and late passage number.

Amniotic fluid derived cells or pancreatic-derived stromal cells were seeded into 24-well tissue culture plates at a density of 5000/cm² and cultured in AMNIOMAX (Invitrogen) or DMEM with 10% FBS respectively under standard cell culture conditions until confluent. After reaching confluence, amniotic fluid derived cells were treated at time 0 hours with a dose of 1.25 μM trichostatin A, and pancreatic-derived stromal cells were treated with a dose of 5.0 μM trichostatin A solubilized in DMSO and medium. At time 48 hours, cell cultures underwent a complete medium change to remove all traces of trichostatin A. At time 96 hours, cell cultures were re-fed with fresh medium and received a second dose of trichostatin A at the same concentration as used previously. At time 120 hours, cell cultures again underwent a complete medium change and were cultured for an additional 24 hours or 148 hours total incubation time. Samples were taken for RT-PCR at the times indicated in Table IX-A to D. RNA samples were obtained daily from the start of the experiment. Culture medium was removed, and cells were washed with PBS then collected in RLT with β-mercaptoethanol (Qiagen). RNA was purified using the RNeasy Mini Kit (Qiagen) and RNA quantity and quality was determined using a spectrophotometer. cDNA was made using the iScript cDNA synthesis kit (BioRad). Human pancreas cDNA was included as a control. Results were normalized against GAPDH expression levels.

Gene expression levels of Sox17, HNF-3 beta, Pdx-1, insulin, and glucagon were analyzed in amniotic-derived cells, while expression levels of glucagon, insulin, HNF-3 beta, and Pdx-1 were analyzed in pancreas-derived cells. Samples of 20 ng cDNA was used in each Real-Time PCR reaction. RT-PCR was performed on the Applied Biosystems 7500, according to the methods described in Example 15. The data were analyzed using the accompanying software.

Prior to treatment with trichostatin A, the amniotic fluid-derived cell line used in Examples 2 & 4 did not express Gata-1 and HNF-3 beta. Insulin and Pdx-1 were expressed at cycle 36 and cycle 37, respectively, as detected by RT-PCR, and Sox-17 expression was detected by RT-PCR at cycle 27. Following treatment with 1.25 μM trichostatin A, Gata-1 gene expression was detected at cycle 37 by RT-PCR but was not detected for the remainder of the experiment. HNF-3 beta gene expression was detected by RT-PCR at cycle 31 following the addition of 1.25 μM trichostatin A but this level of expression decreased to undetectable levels until the second addition of trichostatin A. Following the second treatment with trichostatin A, HNF-3 beta gene expression increased to 35 cycles as detected by RT-PCR. Insulin gene expression was not detectable by RT-PCR following the initial addition of 1.25 μM trichostatin A. Pdx-1 gene expression was detected by RT-PCR at cycle 32 following the addition of 1.25 μM trichostatin A, which decreased following the medium change but increased again after the second treatment with trichostatin A to 37 cycles as detected by RT-PCR. Sox-17 gene expression levels did not change throughout the course of the experiment (Table IX-A). These data provide further support that continued treatment with trichostatin A causes the increase of HNF-3 beta and Pdx-1 gene expression, that a more robust effect on gene expression can be measured with exposure to a higher concentration of trichostatin A for a longer time period, and that insulin gene expression may be inhibited in this cell type by the addition of trichostatin A.

Prior to treatment with trichostatin A, the amniotic fluid-derived cell line used in Example 6 did not express Gata-1, HNF-3 beta and Pdx-1 prior to trichostatin A treatment. Sox-17 and insulin were expressed at cycles 27 and cycles 35, respectively, as detected by RT-PCR, prior to trichostatin A treatment. Following treatment with 1.25 μM trichostatin A, Gata-1 expression was detected at cycle 38 by RT-PCR, though this did not persist for more than 24 hours. Gata-1 expression was again detected at cycle 38 by RT-PCR following the second treatment with trichostatin A. HNF-3 beta expression was detected by RT-PCR at cycle 34 for the first 72 hours with a decrease to cycle 37 by 96 hours and an increase to cycle 33 following the second treatment with trichostatin A. Insulin expression was undetectable following treatment with 1.25 μM trichostatin A, and expression did not change for the duration of the experiment. Pdx-1 expression was detected at cycle 35 by RT-PCR following the initial 48-hour treatment with 1.25 μM trichostatin A. This level was undetectable once trichostatin A was removed but was detected by RT-PCR at cycle 34 following the second treatment of trichostatin A. Sox-17 gene expression levels did not change over the course of the experiment (Table IX-B). These data provide further support that continued treatment with trichostatin A causes an increase of HNF-3 beta and Pdx-1 gene expression, that a more robust effect on gene expression can be measured with exposure to a higher concentration of trichostatin A for a longer time period, and that insulin gene expression may be inhibited in this cell type by the addition of trichostatin A.

Early passage (P5) pancreatic-derived stromal cells expressed Pdx-1 at cycle 38 as detected by RT-PCR but do not express glucagon, HNF-3 beta and insulin prior to treatment with 5.0 μM trichostatin A. Following treatment with trichostatin A, glucagon gene expression was detected by RT-PCR at cycle 35. This gene expression level increased to cycle 33 as detected by RT-PCR at 48 hours but decreased to 38 cycles after the trichostatin A was removed. Following the second addition of trichostatin A, glucagon gene expression increased to 37 cycles. HNF-3 beta gene expression was detectable at 34 cycles by RT-PCR following trichostatin A treatment, and this level of expression persisted until the removal of trichostatin A. When trichostatin A was added to the cells, expression was detected by RT-PCR at cycle 33. Pdx-1 gene expression was not detectable following the initial treatment with trichostatin A but was detected by RT-PCR at cycle 32 at 48 hours. Once trichostatin A was removed from the medium, gene expression was undetectable (Table IX-C). Pdx-1 gene expression was detected at 32 cycles following the second addition of trichostatin as measured by RT-PCR. Insulin gene expression was undetectable while trichostatin A remained in the medium but was detected at 33 cycles by RT-PCR following the medium change. After trichostatin A was added to the medium again, insulin gene expression was undetectable (Table IX-C). These data provide further support that continued treatment with trichostatin A causes an increase of HNF-3 beta and Pdx-1 gene expression that a more robust effect on gene expression can be measured with exposure to a higher concentration of trichostatin A for a longer time period, and that insulin gene expression may be inhibited in this cell type by the addition of trichostatin A.

Results for late passage (P11) pancreatic-derived stromal were similar to those recorded for early passage (P5) pancreatic-derived stromal cells. No genes of interest were detectable prior to treatment with 5.0 μM trichostatin A, but after addition of trichostatin A, glucagon was detected at cycle 33 by RT-PCR and HNF-3 beta was detected at 32 cycles by RT-PCR. Pdx-1 was detected by RT-PCR at cycle 31. Insulin gene expression did not change throughout the course of the experiment. Glucagon gene expression was detected at 33 cycles by RT-PCR for 48 hours following initial treatment with 5.0 μM trichostatin A. This level of glucagon gene expression decreased to 37 cycles following the removal of trichostatin A and was undetectable prior to second treatment with trichostatin A, at which point it was detected at cycle 35 by RT-PCR. HNF-3 beta and Pdx-1 gene expression followed the same pattern. Expression was detected for 48 hours following addition of 5.0 μM trichostatin A at cycles 33 and 32 respectively but was undetectable by RT-PCR after the medium was changed and trichostatin A was removed. Once trichostatin A was added again, HNF-3 beta gene expression increased to 34 cycles and Pdx-1 gene expression increased to 33 cycles as detected by RT-PCR (Table IX-D). These data provide further support that continued treatment with trichostatin A causes an increase of HNF-3 beta and Pdx-1 gene expression, that a more robust effect on gene expression can be measured with exposure to a higher concentration of trichostatin A for a longer time period, and that insulin gene expression may be inhibited in this cell type by the addition of trichostatin A.

### EXAMPLE 10

### Effects of a 6 Hour Trichostatin A Treatment on Gene Expression in Amniotic Fluid-Cells and Pancreatic-Derived Stromal Cells

Amniotic fluid derived cells or pancreas-derived cells were seeded into 24-well tissue culture plates at a density of 5000/cm² and cultured in AMNIOMAX (Invitrogen) or DMEM with 10% FBS respectively under standard cell culture conditions until confluency was reached. Upon reaching confluency, amniotic fluid derived cells were treated with 1.25 μM trichostatin A and pancreas-derived stromal cells were treated with 5.0 μM trichostatin A. The media was changed 6 hours following the addition of trichostatin A and cultures were maintained for the remainder of the experiment. Several cell lines obtained from amniotic fluid (see Example 14) and pancreas (see Example 13) were tested. Samples were taken for RT-PCR at the times indicated in Table XA-D.

RNA samples were obtained at the time the trichostatin A was removed and 24 hours from the start of the experiment. The culture media was removed and cells were washed with PBS then collected in RLT Lysis Buffer with β-mercaptoethanol (Qiagen). RNA was purified using the RNeasy Mini Kit (Qiagen) and RNA quantity and quality was determined using a spectrophotometer. cDNA was made using the iScript cDNA synthesis kit (BioRad). Human pancreas cDNA was included as a control. Results were normalized against GAPDH expression levels.

The expression levels of Sox17, HNF-3 beta, Pdx-1, Insulin, and Gata-1 were analyzed in amniotic-derived cells while the expression levels of glucagon, insulin, PDX-1 and HNF-3 beta were analyzed in pancreas-derived cells. 20 ng of cDNA was used in each RT-PCR reaction, which was performed on the Applied Biosystems 7500, according to the methods described in Example 15. Data was analyzed using the accompanying software.

Prior to treatment with trichostatin A, the amniotic fluid-derived cell line used in Examples 2 & 4 did not express Gata-1 and HNF-3 beta. Following treatment with 1.25 μM trichostatin A, HNF-3 beta expression was detected by RT-PCR cycle 32, but following the wash, increased to 35 cycles. Insulin gene expression decreased from 36 cycles to undetectable levels as determined by RT-PCR following the addition of trichostatin A (Table X-A). Pdx-1 expression was detected at cycle 37 by RT-PCR prior to treatment with trichostatin A. This level of expression decreased to 35 cycles after treatment with trichostatin A but was undetectable once trichostatin A was removed. Expression of Gata-1 and sox-17 remained unchanged with the addition of trichostatin A (Table X-A).

Prior to treatment with trichostatin A, the amniotic fluid-derived cell line used in Example 6 did not express Gata-1, HNF-3 beta or Pdx-1. Insulin and sox-17 were expressed at cycle 35 and cycle 27 respectively by RT-PCR. 6 hours of treatment with 1.25 μM trichostatin A was sufficient to increase the expression of HNF-3 beta and Pdx-1 to cycles 31 and 35 respectively, as detected by RT-PCR. Following removal of trichostatin A, HNF-3 beta expression was detected at cycle 35 by RT-PCR and Pdx-1 was undetectable. Insulin gene expression was not detectable following addition of trichostatin A. Gata-1 and Sox-17 gene expression levels remained unchanged following treatment of 1.25 μM trichostatin A (Table X-B). These data provide support that insulin gene expression may be inhibited by trichostatin A treatment in these cells and that 6 hours of treatment was sufficient to see an increase in gene expression of HNF-3 beta and Pdx-1.

Early passage (P5) pancreatic-derived stomal cells did not express glucagon, HNF-3 beta or insulin prior to addition of 5.0 μM trichostatin A. Following trichostatin A treatment glucagon gene expression was detected by RT-PCR at cycle 37, HNF-3 beta gene expression was detected by RT-PCR at cycle 35. Following trichostatin A removal, glucagon gene expression remained the same but HNF-3 beta was undetectable. Insulin gene expression remained undetectable until the trichostatin A was removed from the media, where it was then detected at cycle 32 by RT-PCR. Pdx-1 gene expression was detected at 38 cycles by RT-PCR prior to trichostatin A treatment, but following treatment that level of expression increased to 35 cycles (Table XC). These data suggest that treatment with 5.0 μm trichostatin A for 6 hours was not sufficient for increasing expression of Pdx-1, but continues to suppress insulin gene expression as noted previously.

Late passage (P11) pancreatic-derived stromal cells did not express any of the genes of interest prior to treatment with 5.0 μM trichostatin A. Following trichostatin A treatment, glucagon was detected at 37 cycles, HNF-3 beta was detected at 36 cycles and Pdx-1 was detected at 37 cycles by RT-PCR. There was no change in insulin gene expression following the addition of trichostatin A. Following the removal of trichostatin A, glucagon gene expression was detected at 35 cycles by RT-PCR, while HNF-3 beta and Pdx-1 gene expression was undetectable (Table X-D). These data suggests that trichostatin A is necessary to up-regulate HNF-3 beta and Pdx-1 gene expression.

### EXAMPLE 11

### Dose Titration of Chromatin Remodeling or DNA Demethylating Agents at Various Times

Amniotic fluid or pancreatic progenitor cells will be plated in duplicate culture plates with multiple replicate sets. After reaching confluency (2-3 days), either trichostatin A (or an alternative histone deacetylase inhibitor) or 5-azacytidine (or an alternative demethylating agent) will be added to each replicate set at a range of 0.001 μM to 50 mM, final concentration. An equivalent amount of solvent will be added to the no treatment control cultures. Cells will be returned to standard culture conditions for a time period of 6 hr, 12 hr, 24 hr, or 48 hr. After the appropriate time period is concluded, one plate will be treated with a metabolic dye, for example, MTS, to monitor cell viability as per manufacturer's instructions. For the other matched culture plate, medium will be removed, cells will be washed with phosphate buffered saline (PBS), and RLT lysis buffer containing β-mercaptoethanol (Qiagen) will be added to each well. Samples will be homogenized using Qiashredder columns (Qiagen) and RNA will be purified using the RNeasy Mini Kit (Qiagen). RNA quantity and quality will be determined using a spectrophotometer, and cDNA will be made using the iScript cDNA synthesis kit (BioRad). Samples of 20 ng cDNA will be used in each reaction to determine expression levels of Sox17, HNF-3 beta, Pdx-1, insulin, and glucagon. Real-Time PCR will be performed on an Applied Biosystems 7500 system, and data will be analyzed using the accompanying software.

### EXAMPLE 12

### Changes in Gene Expression by the Simultaneous Addition of a Histone Deacetylase Inhibitor and a DNA Demethylating Agent

Amniotic fluid or pancreatic progenitor cells will be plated and allowed to reach confluency (2-3 days). Both trichostatin A (or an alternative histone deacetylase inhibitor) and 5-azacytidine (or an alternative demethylating agent) will be added to the culture at an optimal, nontoxic concentration and for a preferred time period to induce appropriate gene expression, as determined from Examples 1-10 above. At the conclusion of the time period (for example, 24 hours), medium will be removed, cells will be washed with phosphate buffered saline (PBS), and RLT lysis buffer containing β-mercaptoethanol (Qiagen) will be added to each well. Samples will be homogenized using Qiashredder columns (Qiagen) and RNA will be purified using the RNeasy Mini Kit (Qiagen). RNA quantity and quality will be determined using a spectrophotometer, and cDNA will be made using the iScript cDNA synthesis kit (BioRad). Samples of 20 ng cDNA will be used in each reaction to determine expression levels of Sox17, HNF-3 beta, Pdx-1, insulin, and glucagon. Real-Time PCR will be performed on an Applied Biosystems 7500 system, and data will be analyzed using the accompanying software.

### EXAMPLE 13

### The Establishment of Human Pancreatic Cell Lines

Pancreas Preparation—Human pancreata not suitable for clinical transplantation were obtained from The National Disease Research Interchange (Philadelphia, Pa.) following appropriate consent for research use. The pancreas was transferred with organ preservation solution to a stainless steel pan on ice and trimmed of all extraneous tissue. The pancreatic duct was cannulated with an 18 gauge catheter and the pancreas was injected with an enzyme solution, which contained the LIBERASE HI™ enzyme (Roche 0.5 mg/ml, Roche) and DNase I (0.2 mg/ml) dissolved in Dulbecco's Phosphate Buffered Saline (DPBS).

Rapid Mechanical Dissociation Followed by Enzymatic Digestion—The enzyme infused pancreata were homogenized in a tissue processor, pulsed 3-5 times for 3-5 seconds/pulse, and the dissociated tissue was transferred to two 500 ml trypsinizing flasks (Bellco) containing magnetic stir bars. Thereafter, 50-100 ml of the enzyme solution was added to each flask. The flasks were placed in a 37° C. water bath on submersible stir plates and allowed to incubate with an intermediate stir rate for 10 minutes. The stirring was stopped, and the finely digested tissue was removed from the flask and transferred into a 250 ml tube containing DPBS, 5% Fetal Bovine Serum (FBS) and 0.1 mg/ml DNase I (DPBS+) at 4° C. to quench the digestion process. The flasks were replenished with 50-100 ml of the enzyme solution and returned to the water bath, and the stirring was re-initiated for an additional ten minutes. Again, the flasks were removed and the fine digest was collected and transferred to the 250 ml tubes on ice. This process was repeated for an additional 3-5 times until the pancreas was completely digested.

Gradual Mechanical Dissociation with Simultaneous Enzyme Digestion—The enzyme infused pancreata were processed according to methods as described in Diabetes 37:413-420 (1988). Briefly, the pancreata were cleaned of extraneous tissue and injected with the enzyme solution as described above. The pancreata were then placed into a Ricordi Chamber with beads and covered with a screen with a mesh size of 400-600 μm to retain larger clusters of tissue. The chamber was covered; the enzyme solution was circulated through the chamber at approximately 37° C., and the chamber was shaken to allow beads to disrupt pancreatic tissue during enzymatic digestion. Once adequate dissociation and digestion was achieved, the digestion was terminated and the tissue was collected.

Tissue Separation—The collected tissue was centrifuged at 150×g for 5 minutes at 4° C. The supernatant was aspirated and the tissue was washed two additional times in DPBS+. Following the final wash, the tissue was applied to a discontinuous gradient for purification. The digested tissue was suspended in polysucrose (Mediatech, VA) with a density of 1.108 g/ml at a ratio of 1-2 ml tissue pellet per 10 ml of polysucrose solution. The tissue suspension was then transferred to round-bottom polycarbonate centrifuge tubes, and polysucrose solutions with densities of 1.096 and 1.037 were carefully applied to the tubes. A final layer of DMEM completed the discontinuous purification gradient. The gradient tubes were centrifuged at 2000 rpm for 20 minutes at 4° C. with no brake applied. Following centrifugation, the tissue was collected from each interface (three interfaces total), washed several times in DPBS+ as described above, and collected in a 50 ml test tube.

Further Cell Cluster Dissociation—Optionally, one can further dissociate large cell clusters obtained using the above protocol into smaller clusters or single cell suspensions. After the final wash, the tissue from each fraction was suspended in 10 ml 1× trypsin/EDTA solution containing 200 U/mL DNase I. The tubes were placed in the water bath and repeatedly aspirated and discharged from a 10 ml serological pipette for 5-6 minutes until a near single cell suspension was achieved. The digestion was quenched with the addition of 4° C. DPBS+ and the tubes centrifuged at 800 rpm for 5 minutes. The cell suspensions were washed with DPBS+ and cultured as described below.

Pancreatic Cell Culture—Following the final wash, the cells from each interface were resuspended in DMEM, 2% FBS, 100 U/μg penicillin/streptomycin, ITS, 2 mM L-Glutamine, 0.0165 mM ZnSO₄ (Sigma), and 0.38 μM 2-mercaptoethanol (Invitrogen, CA) (hereinafter “the selection medium”). Six ml of the cell suspension was seeded in T-25 tissue culture flasks and 12 ml of the cell suspension was seeded into T-75 flasks. The flasks were placed in 37° C. incubators with 5% CO₂. Following 2-4 weeks culture, a complete medium change was performed and adherent cells were returned to culture in DMEM (2750 mg/L D-glucose, 862 mg/L glutamine) (Gibco, CA) with 5% FBS (HyClone, UT), 1% P/S, 0.0165 mM ZnSO₄ (hereinafter “the growth medium”) and allowed to reach near confluence (this stage is referred to as “passage 0” or “P0”), at which point they were passaged. Subsequent culturing of the cells was at 5000 cell/cm² in the growth medium. Cultures were passaged every 7-10 days at ˜70-90% confluency.

### EXAMPLE 14

### The Establishment of Human Amniotic Fluid-Derived Cell Lines

Amniotic, fluid used to isolate the cells of the present invention was taken from samples obtained through routine amniocentesis performed at 17-22 weeks of gestation for fetal karyotyping (Drexel University). The amniotic fluid was centrifuged for 7 minutes at 400×g and the supernatant removed. The resulting cell pellet was resuspended in growth medium. The cells were cultured either on collagen type IV (1 mg/100 mm plate) or fibronectin (10 micrograms/ml) coated plates. The cell yield from AF samples had a large variation (8000-300000 cell/sample), and some samples also contained a significant degree of red blood cell contamination. The cultures were left undisturbed for at least 5-10 days under hypoxic conditions (3% O₂). Thereafter, the cultures were fed with the same growth medium and cultured until the cultures reached 70-80% confluency. Cells at this stage were referred to as “P0”. In some cultures, colonies of cells were isolated using a cloning ring and sub-cultured into a different culture plate. Cells were released from P0 culture by using TrypLE Express™ (Invitrogen) and seeded into fibronectin or collagen type IV coated flaks/dishes/plates at various densities (50-10,000 cell/cm²). Some of the P0 cells were used for serial dilution cloning. The population doubling time of the fastest growing cells was ˜24 hrs at early passages. Cells were typically split at 60% confluency and reseeded at 100-10000 cells/cm².

### EXAMPLE 15

### PCR Analysis of Cells

RNA was extracted from cells cultured in the growth media. Total RNA from human pancreas (Ambion, INC) was included as a positive control.

RNA extraction, purification, and cDNA synthesis. RNA samples were purified through binding to a silica-gel membrane (Rneasy Mini Kit, Qiagen, CA) in the presence of an ethanol-containing, high-salt buffer while contaminants were washed away. High-quality RNA was then eluted in water. Yield and purity were assessed by A260 and A280 readings on the spectrophotometer. cDNA copies were made from purified RNA using the iScript cDNA synthesis kit (BioRad, CA).

Real-time PCR amplifcation and quantitative analysis. Unless otherwise stated, all reagents were purchased from Applied Biosystems. Real-time PCR reactions were performed using the ABI PRISM™ 7500 Sequence Detection System. TAQMAN™ FAST UNIVERSAL PCR MASTER MIX™ (ABI, CA) was used with 20 ng of reverse transcribed RNA in a total reaction volume of 20 μl. Each cDNA sample was run in duplicate to allow correction of pipetting errors. Primers and FAM-labeled TAQMAN™ probes were used at concentrations of 200 nM. The level of expression for each target gene was normalized using the pre-developed Applied Biosystem's human glyceraldehyde-3-phosphate dehydrogenase (GAPDH) endogenous control kit. Primers and probes were designed using either ABI PRISM PRIMER EXPRESS™ software or a pre-developed ABI gene analysis kit. For each gene, either one of the primers or the probe were designed to be exon-boundary spanning. This eliminated the possibility of the primers/probe binding to any genomic DNA present. The primer and probe sets are listed as follows: Pdx-1 (Hs00426216), Insulin (Hs00355773), glucagon (Hs00174967), and FoxA2 (HNF 3-beta) (Hs00232764). The remaining primers were designed using the PRIMERS program (ABI, CA). After an initial 95° C. incubation for 20 sec, samples were cycled 40 times in two stages: a denaturation step at 95° C. for 3 sec, followed by an annealing/extension step at 60° C. for 30 sec.

For each primer/probe set, a cycle time value was determined as the cycle number at which the fluorescence intensity of the PCR reaction reached a specific value in the middle of the exponential region of amplification. An increase in expression of a gene corresponded to a decrease in the number of cycles required for the fluorescence intensity to reach this value.

### EXAMPLE 16

### Fluorescence-Activated Cell Sorting (FACS) Analysis

Adhered cells were removed from culture plates by five-minute incubation with the TRYPLE™ express solution (Gibco, CA). Released cells were resuspended in DMEM supplemented with 10% FBS and recovered by centrifugation, followed by washing and resuspending the cells in a staining buffer consisting of 2% BSA, 0.05% sodium azide (Sigma, MO) in PBS. If appropriate, the cells were Fc-receptor blocked using a 0.1% γ-globulin (Sigma) solution for 15 min. Aliquots (approximately 10⁵ cells) were incubated with either phycoerythirin (PE) or allophycocyanin (APC) conjugated monoclonal antibodies (5 μl antibody per 10⁶ cells), as indicated in Table XI, or with an unconjugated primary antibody. Controls included appropriate isotype matched antibodies, non-stained cells, and cells only stained with secondary conjugated antibody. All incubations with antibodies were performed for 30 mins at 4° C. after which the cells were washed with the staining buffer. Samples that were stained with unconjugated primary antibodies were incubated for an additional 30 mins at 4° C. with secondary conjugated PE or -APC labeled antibodies. See Table XII for a list of secondary antibodies used. Washed cells were pelleted and resuspended in the staining buffer and the cell surface molecules were identified by using a FACS Array (BD Biosciences) by collecting at least 10,000 events.

For intracellular staining, cells were first fixed for 10 mins with 4% paraformaldheyde, followed by two rinses in the staining buffer, centrifugation of cells and resuspension of the cells in a perneabilization buffer containing 0.5% Triton-X (Sigma) in PBS for 5 mins at room temperature (RT). The permeabilized cells were rinsed twice with a rinsing buffer, centrifuged, and resuspended in the staining buffer, and incubated with an appropriate conjugated antibody (5 μl antibody per 10⁶ cells) for 30 mins at 4° C. Samples that were stained with unconjugated primary antibodies were incubated for an additional 30 mins at 4° C. with secondary conjugated PE or -APC labeled antibodies (Table XII). Washed cells were pelleted and resuspended in the staining buffer and the internal proteins were identified by using a FACSArray (BD Biosciences) by collecting at least 10,000 events.

### EXAMPLE 17

### Immunostaining of Cells

10,000 cells/cm² cells were seeded into glass bottom 35 mm microwell dishes (Matek Corp, MA) in growth medium. Following three days in culture, the cells were fixed for 10 mins with 4% paraformaldheyde, followed by two rinses in PBS and addition of a permeabilization buffer containing 0.5% Triton-X (Sigma) for 5 mins at room temperature (RT), followed by an additional three rinses with PBS. The fixed and permeabilized cells were blocked with either 1% bovine serum albumin (BSA) or 4% serum from the species where the secondary antibody was raised in (goat, donkey, or rabbit). Control samples included reactions with the primary antibody omitted or where the primary antibody was replaced with corresponding immunoglobulins at the same concentration as the primary antibodies. Stained samples were rinsed with a PROLONG® antifade reagent (Invitrogen, CA) containing diamidino-2-phenylindole, dihydrochloride (DAPI) to counter-stain the nucleus. Images were acquired using a Nikon Confocal Eclipse C-1 inverted microscope (Nikon, Japan) and a 60× objective.

### EXAMPLE 18

### The Effects of Trichostatin A Treatment and Inhibition of the Sonic Hedgehog Pathway on Gene Expression in Early Passage (P5) Pancreatic-Derived Stromal Cells and Amniotic Fluid-Derived Cells

Pancreatic-derived stromal cells are obtained according to the methods described in Example 13. Cells are seeded into a 24-well tissue culture plate at a density of 5000/cm2/well and cultured in DMEM with 10% FBS under standard cell culture conditions until confluent. Amniotic fluid obtained from National Disease Research Interchange (NDRI) is processed according to the methods described in Example 14. Cells are seeded into a 24-well tissue culture plate at a density of 5000/cm2/well and cultured in AMNIOMAX (Invitrogen) under standard cell culture conditions until confluent. After the cells reach confluency, sample wells are treated with 1.25 mM trichostatin A diluted in DMSO and medium; control wells receive DMSO at an equivalent concentration. At 24 hours, sample wells receive another dose of 1.25 μM trichostatin A. A 10-μM dose of Cyclopamine (Sigma) and 10 mM Nicotinamide (Sigma) are added to the cell culture medium. The following day, medium is removed and cells are washed with PBS. New medium is added once every other day including similar doses of Nicotinamide and Cyclopamine. On day 7, Cells are collected for real time PCR analysis as described in Example 15.

### EXAMPLE 19

### The Effect of Trichostatin A on Gene Expression in Peripheral Blood Mononuclear Cells

Peripheral blood mononuclear cells will be isolated by density gradient sedimentation and plated in culture at a density of 0.5-2×10⁶ per ml. An activation mitogen, such as for example PHA, will be added at a final concentration of 10 ug/ml. Controls will omit the addition of PHA. Cells will be cultured for 3 days after which cells will be collected, washed, counted, resuspended and replated at a density of 1-2×10⁶ per ml. Trichostatin A (or an alternative histone deacetylase inhibitor) and/or 5-azacytidine (or an alternative demethylating agent) will be added to the culture at an optimal, nontoxic concentration and for a preferred time period to induce appropriate gene expression, as determined from Examples 1-10 above. Untreated control wells will receive a similar dilution of vehicle or diluent. At the conclusion of the culture time period (for example, 24 hours), medium will be removed, and cells will be washed with phosphate buffered saline (PBS). RLT lysis buffer containing β-mercaptoethanol (Qiagen) will be added to each well. Samples will be homogenized using Qiashredder columns (Qiagen) and RNA will be purified using the RNeasy Mini Kit (Qiagen). RNA quantity and quality will be determined using a spectrophotometer, and cDNA will be made using the iScript cDNA synthesis kit (BioRad). Samples of 20 ng cDNA will be used in each reaction to determine expression levels of PDX-1, insulin, glucagon, somatostatin, sox17, gata4, globin, beta-2-microglobulin. Real-Time PCR will be performed on an Applied Biosystems 7500 system, and data will be analyzed using the accompanying software.

### EXAMPLE 20

### The Effects of Trichostatin A Treatment on Gene Expression in Resting Peripheral Blood Mononuclear Cells (PBMCs) and PBMCs Treated With the Mitogenic Lectin PHA

Human peripheral blood mononuclear cells (PBMCs) were isolated from whole blood using Histopaque (Sigma) gradients and standard density centrifugation. PBMCs are a heterogeneous mixture of lymphoid cells including quiescent T-lymphocytes. Cells were washed thoroughly, counted, and resuspended at 1-2×10⁶ cells per ml in culture medium containing RPMI-1640 and 10% FCS. Phytohemagglutinin (PHA; Sigma) is a mitogenic lectin that specifically induces T-lymphocyte activation and proliferation. PHA was added to the cell suspension at a final concentration of 10 μg/ml, and PBMCs were cultured for 3 days at 37° C. with 5% CO₂. This resulted in the activation of T-lymphocytes. At the end of culture, PBMCs were pooled to harvest, washed thoroughly, and resuspended in fresh culture medium with 5 μM trichostatin A diluted in DMSO. A control culture of PHA-treated PBMCs received an equivalent dilution of DMSO alone. Cells were returned to culture for an additional 24 or 48 hours. Alternatively, resting PBMCs were isolated in a similar manner and cultured for 24 or 48 hours total incubation time with either 5 μM trichostatin A or DMSO alone. At the conclusion of culture, cells were harvested and washed with PBS prior to preparation of RNA in RLT lysis buffer with β-mercaptoethanol (Qiagen). RNA was purified using the RNeasy Mini Kit (Qiagen); RNA quantity and quality were determined using a spectrophotometer. CDNA was made using the iScript CDNA synthesis kit (BioRad). Samples of 20 ng CDNA were used in each RT-PCR reaction, performed on the Applied Biosystems 7500. Results were normalized against GAPDH expression levels with data analysis performed using the accompanying software. The results are displayed in Table XIII.

Sox17, HNF-3 beta, insulin, somatostatin, and glucagon were not expressed by resting PBMCs or PHA treated PBMCs containing activated T-lymphocytes. Expression of these genes remained negative after trichostatin A treatment (see Table XIII).

GATA1 is a hematopoietic lineage marker that was detectable in both resting and PHA treated PBMCs in the absence of trichostatin A treatment. However, GATA1 expression decreased below detectable levels after 24 or 48 hours treatment with 5 μM trichostatin A. In this case, application of the chromatin-remodeling agent trichostatin A decreased expression of a differentiation-related gene associated with the hematopoietic lineage (see Table XIII).

GATA4 is a marker of mesenchymal and/or endodermal lineage differentiation. Resting PBMCs failed to express GATA4 but acquired weak expression after trichostatin A treatment for 24 or 48 hours. PHA treated PBMCs containing activated T-lymphocytes treated with 5 μM trichostatin A for either 24 or 48 hours showed strong up-regulation of GATA4 (see Table XIII). These data suggest that differentiated cells of the hematopoietic lineage can be induced to express markers of other differentiated cell or tissue lineages after treatment with trichostatin A.

PDX-1 expression was undetectable in resting PBMCs either with or without trichostatin A treatment. PDX-1 expression was also undetectable in PHA treated PBMCs containing activated T-lymphocytes that were not treated further with trichostatin A. However, a consistent low level of expression of PDX-1 was noted in PHA treated PBMCs after 24 or 48 hours exposure to trichostatin A (see Table XIII). These data suggest that actively dividing cells and/or mitogenic activation is required to act in concert with chromatin remodeling agents to promote expression of some alternative lineage genes in these cells.

TABLE I
THE EFFECTS OF HISTONE DEACETYLASE INHIBITOR
TREATMENT ON GENE EXPRESSION IN PANC-1 CELLS AND NEONATAL
FIBROBLASTS.

| | GAPDH | glucagon | HNF-3beta | Insulin | PDX-1 | Sox17 |
|---|---|---|---|---|---|---|
| untreated fibroblasts | +++ | nd | nd | nd | nd | nd |
| fibroblasts with 2.5 μM | +++ | +++ | +++ | nd | + | +++ |
| trichostatin A after 48 | | | | | | |
| hours | | | | | | |
| Fibroblasts with 5.0 μM | +++ | +++ | +++ | nd | + | +++ |
| trichostatin A after 48 | | | | | | |
| hours | | | | | | |
| untreated Panc-1 | +++ | nd | +++ | nd | +++ | +++ |
| Pane-1 with 2.5 μM | +++ | +++ | +++ | + | +++ | +++ |
| trichostatin A after 48 | | | | | | |
| hours | | | | | | |
| pane-1 with 5.0 μM | +++ | +++ | +++ | + | +++ | +++ |
| trichostatin A after 48 | | | | | | |
| hours | | | | | | |
| human pancreas | +++ | +++ | +++ | ++ | +++ | +++ |

SE standard error
nd not detectable at >40 cycles by RT-PCR
+ detectable at greater than 35–40 cycles by RT-PCR
++ detectable at less than 35 cycles by RT-PCR with SE greater than 0.9
+++ detectable at less than 35 cycles by RT-PCR with SE less than 0.9
All RT-PCR was performed on the Applied Biosystems 7500 Real-Time PCR System

TABLE II-A
GENE EXPRESSION IN THE UNTREATED AMNIOTIC
FLUID-DERIVED CELL LINE AFCA009-A.

| | Untreated | | | | |
|---|---|---|---|---|---|
| Time (hours) | glucagon | HNF-3beta | Insulin | PDX-1 | Sox17 |
| 0 | nd | nd | + | nd | +++ |
| 0.5 | nd | + | + | nd | +++ |
| 1.5 | nd | nd | + | nd | +++ |
| 3 | nd | nd | +++ | nd | +++ |
| 6 | nd | + | +++ | nd | +++ |
| 12 | nd | nd | +++ | nd | +++ |
| 24 | nd | nd | +++ | nd | +++ |

nd not detectable at >40 cycles by RT-PCR
+ detectable between 35 and 40 cycles by RT-PCR
++ detectable at less than 35 cycles by RT-PCR with std error greater than 0.9
+++ detectable at less than 35 cycles by RT-PCR with std error less than 0.9
All RT-PCR was performed on the Applied Biosystems 7500 Real-Time PCR System

TABLE II-B
GENE EXPRESSION IN THE AMNIOTIC FLUID-DERIVED
CELL LINE AFCA009-A TREATED WITH TRICHOSTATIN A
FOR 24 HOURS.

| | Treated | | | | |
|---|---|---|---|---|---|
| Time (hours) | glucagon | HNF-3beta | Insulin | PDX-1 | Sox17 |
| 0.5 | nd | + | + | nd | +++ |
| 1.5 | nd | + | + | nd | +++ |
| 3 | nd | +++ | + | nd | +++ |
| 6 | nd | +++ | nd | + | +++ |
| 12 | + | +++ | + | +++ | +++ |
| 24 | +++ | +++ | + | +++ | +++ |

nd not detectable at >40 cycles by RT-PCR
+ detectable between 35 and 40 cycles by RT-PCR
++ detectable at less than 35 cycles by RT-PCR with std error greater than 0.9
+++ detectable at less than 35 cycles by RT-PCR with std error less than 0.9
All RT-PCR was performed on the Applied Biosystems 7500 Real-Time PCR System

TABLE III-A
GENE EXPRESSION IN LATE PASSAGE
PANCREATIC-DERIVED STROMAL CELLS.

| | untreated | | | | |
|---|---|---|---|---|---|
| Time (hours) | glucagon | HNF-3beta | Insulin | PDX-1 | Sox17 |
| 0 | nd | nd | nd | nd | + |
| 0.5 | nd | nd | nd | nd | nd |
| 1.5 | nd | nd | nd | nd | nd |
| 3 | nd | nd | nd | nd | + |
| 6 | nd | nd | nd | nd | + |
| 12 | nd | nd | nd | nd | nd |
| 24 | nd | nd | nd | nd | + |

nd not detectable at >40 cycles by RT-PCR
+ dectable between 35 and 40 cycles by RT-PCR
++ dectable at less than 35 cycles by RT-PCR with std error greater than 0.9
+++ detectable at less than 35 cycles by RT-PCR with std errorless than 0.9
All RT-PCR was performed on the Applied Biosystems 7500 Real-Time PCR System

TABLE III-B
GENE EXPRESSION IN LATE PASSAGE PANCREATIC-
DERIVED STROMAL CELLS TREATED WITH
TRICHOSTATIN A FOR 24 HOURS.

| | Treated | | | | |
|---|---|---|---|---|---|
| Time (hours) | glucagon | HNF-3beta | Insulin | PDX-1 | Sox17 |
| 0.5 | nd | nd | nd | nd | + |
| 1.5 | nd | nd | nd | nd | nd |
| 3 | nd | nd | nd | nd | + |
| 6 | nd | + | nd | nd | + |
| 12 | nd | + | nd | + | + |
| 24 | + | +++ | nd | +++ | +++ |

nd not detectable at >40 cycles by RT-PCR
+ detectable between 35 and 40 cycles by RT-PCR
++ detectable at less than 35 cycles by RT-PCR with std error greater than 0.9
+++ detectable at less than 35 cycles by RT-PCR with std error less than 0.9
All RT-PCR was performed on the Applied Biosystems 7500 Real-Time PCR System

TABLE IV
THE EFFECT OF CHRONIC TRICHOSTATIN A TRATMENT ON
GENE EXPRESSION IN AMNIOTIC FLUID-DERIVED CELLS.

| 24 hours | glucagon | HNF-3beta | Insulin | PDX-1 | Sox17 |
|---|---|---|---|---|---|
| untreated | nd | nd | + | nd | +++ |
| 500 nM | + | +++ | nd | +++ | +++ |
| 1 □M | +++ | +++ | nd | +++ | +++ |
| 48 hours | glucagon | HNF-3b | Insulin | PDX-1 | Sox17 |
| untreated | nd | + | +++ | nd | +++ |
| 500 nM | + | +++ | nd | + | +++ |
| 1 μM | ++ | +++ | nd | +++ | +++ |
| 72 hours | glucagon | HNF-3b | Insulin | PDX-1 | Sox17 |
| untreated | nd | + | +++ | nd | +++ |
| 500 nM | + | +++ | nd | +++ | +++ |
| 1 μM | +++ | +++ | nd | +++ | +++ |

nd not detectable at >40 cycles by RT-PCR
+ detectable between 35 and 40 cycles by RT-PCR
++ detectable at less than 35 cycles by RT-PCR with std error greater than 0.9
+++ detectable at less than 35 cycles by RT-PCR with std error less than 0.9
All RT-PCR was performed on the Applied Biosystems 7500 Real-Time PCR System

TABLE V
THE EFFECT OF CHRONIC TRICHOSTATIN A TRATMENT ON
GENE EXPRESSION IN LATE PASSAGE PANCREATIC-
DERIVED STROMAL CELLS.

| 24 hours | glucagon | HNF-3beta | Insulin | PDX-1 | Sox17 |
|---|---|---|---|---|---|
| untreated | nd | nd | nd | nd | nd |
| 1.25 μM | + | +++ | nd | + | +++ |
| 2.5 μM | nd | + | nd | +++ | +++ |
| 48 hours | glucagon | HNF-3b | Insulin | PDX-1 | Sox17 |
| untreated | nd | nd | nd | nd | + |
| 1.25 μM | + | ++ | nd | +++ | +++ |
| 2.5 μM | + | + | nd | +++ | +++ |
| 72 hours | glucagon | HNF-3b | Insulin | PDX-1 | Sox17 |
| untreated | nd | nd | nd | nd | + |
| 1.25 μM | + | +++ | nd | +++ | +++ |
| 2.5 μM | nd | +++ | nd | + | +++ |

nd not detectable at >40 cycles by RT-PCR
+ detectable between 35 and 40 cycles by RT-PCR
++ detectable at less than 35 cycles by RT-PCR with std error greater than 0.9
+++ detectable at less than 35 cycles by RT-PCR with std error less than 0.9
All RT-PCR was performed on the Applied Biosystems 7500 Real-Time PCR System

TABLE VI-A
THE EFFECT OF A SINGLE CHRONIC TRICHOSTATIN A
DOSE ON GENE EXPRESSION IN AN AMNIOTIC
FLUID-DERIVED CELL LINE.

| sample | Gata1 | HNF | insulin | Pdx-1 | sox17 |
|---|---|---|---|---|---|
| untreated | nd | nd | + | + | +++ |
| 24 hrs | nd | +++ | nd | + | +++ |
| 48 hrs | nd | +++ | nd | nd | +++ |
| 72 hrs | nd | + | + | nd | +++ |
| 96 hrs | nd | nd | + | nd | +++ |
| 120 hrs | nd | + | + | nd | +++ |
| 144 hrs | nd | nd | + | nd | +++ |

nd not detectable at >40 cycles by RT-PCR
+ detectable between 35 cycles and 40 cycles by RT-PCR
++ detectable at less than 35 cycles by RT-PCR with SE greater than 0.9
+++ detectable at less than 35 cycles by RT-PCR with SE less than 0.9
All RT-PCR was performed on the Applied Biosystems 7500 Real-Time PCR System

TABLE VI-B
THE EFFECT OF A SINGLE CHRONIC TRICHOSTATIN A
DOSE ON GENE EXPRESSION IN A SECOND AMNIOTIC
FLUID-DERIVED CELL LINE.

| sample | Gata1 | HNF | insulin | Pdx-1 | sox17 |
|---|---|---|---|---|---|
| untreated | nd | nd | +++ | nd | +++ |
| 24 hrs | nd | +++ | nd | + | +++ |
| 48 hrs | nd | +++ | nd | nd | +++ |
| 72 hrs | nd | + | + | nd | +++ |
| 96 hrs | nd | + | ++ | nd | +++ |
| 120 hrs | nd | + | ++ | nd | +++ |
| 144 hrs | nd | + | ++ | nd | +++ |

nd not detectable at >40 cycles by RT-PCR
+ detectable between 35 and 40 cycles by RT-PCR
++ detectable at less than 35 cycles by RT-PCR with SE greater than 0.9
+++ detectable at less than 35 cycles by RT-PCR with SE less than 0.9
All RT-PCR was performed on the Applied Biosystems 7500 Real-Time PCR System

TABLE VI-C
THE EFFECT OF A SINGLE CHRONIC TRICHOSTATIN A
DOSE ON GENE EXPRESSION IN AN EARLY PASSAGE
(P5) PANCREATIC-DERIVED STROMAL CELL LINE.

| sample | glucagon | HNF | Insulin | Pdx-1 |
|---|---|---|---|---|
| untreated | nd | nd | nd | + |
| 24 hrs | +++ | +++ | nd | +++ |
| 48 hrs | + | nd | nd | nd |
| 72 hrs | + | nd | nd | nd |
| 96 hrs | +++ | nd | nd | nd |
| 120 hrs | nd | nd | nd | nd |
| 144 hrs | nd | nd | nd | nd |

nd not detectable at >40 cycles by RT-PCR
+ detectable between 35 and 40 cycles by RT-PCR
++ detectable at less than 35 cycles by RT-PCR with SE greater than 0.9
+++ detectable at less than 35 cycles by RT-PCR with SE less than 0.9
All RT-PCR was performed on the Applied Biosystems 7500 Real-Time PCR System

TABLE VI-D
THE EFFECT OF A SINGLE CHRONIC TRICHOSTATIN A
DOSE ON GENE EXPRESSION IN AN LATE PASSAGE
(P14) PANCREATIC-DERIVED STROMAL CELL LINE.

| sample | glucagon | HNF | Insulin | Pdx-1 |
|---|---|---|---|---|
| untreated | nd | nd | nd | nd |
| 24 hrs | +++ | + | nd | +++ |
| 48 hrs | + | nd | nd | nd |
| 72 hrs | nd | nd | nd | nd |
| 96 hrs | nd | nd | nd | nd |
| 120 hrs | nd | nd | nd | nd |
| 144 hrs | nd | nd | nd | nd |

nd not detectable at >40 cycles by RT-PCR
+ detectable between 35 and 40 cycles RT-PCR
++ detectable at less than 35 cycles RT-PCR with SE greater than 0.9
+++ detectable at less than 35 cycles RT-PCR with SE less than 0.9
All RT-PCR was performed on the Applied Biosystems 7500 Real-Time PCR System

TABLE VII-A
THE EFFECT OF TWO 500 NM TRICHOSTATIN A DOSES ON
GENE EXPRESSION IN AN AMNIOTIC
FLUID-DERIVED CELL LINE.

| sample | Gata1 | HNF | insulin | Pdx-1 | sox17 |
|---|---|---|---|---|---|
| untreated | nd | nd | + | + | +++ |
| 24 hrs | nd | +++ | nd | + | +++ |
| 48 hrs | nd | +++ | + | nd | +++ |
| 72 hrs | nd | + | + | nd | +++ |
| 96 hrs | nd | nd | + | nd | +++ |
| 120 hrs | nd | + | + | nd | +++ |
| 144 hrs | nd | + | + | nd | +++ |

nd not detectable at >40 cycles by RT-PCR
+ detectable between 35 and 40 cycles by RT-PCR
++ detectable at less than 35 cycles by RT-PCR with SE greater than 0.9
+++ detectable at less than 35 cycles by RT-PCR with SE less than 0.9
All RT-PCR was performed on the Applied Biosystems 7500 Real-Time PCR System

TABLE VII-B
THE EFFECT OF TWO 500 NM TRICHOSTATIN A DOSES ON
GENE EXPRESSION IN A SECOND AMNIOTIC
FLUID-DERIVED CELL LINE.

| sample | Gata1 | HNF | insulin | Pdx-1 | sox17 |
|---|---|---|---|---|---|
| untreated | nd | nd | +++ | nd | +++ |
| 24 hrs | nd | +++ | nd | + | +++ |
| 48 hrs | nd | +++ | nd | nd | +++ |
| 72 hrs | nd | +++ | +++ | nd | +++ |
| 96 hrs | nd | + | +++ | nd | +++ |
| 120 hrs | nd | + | + | nd | +++ |
| 144 hrs | nd | + | +++ | nd | +++ |

nd not detectable at >40 cycles by RT-PCR
+ detectable between 35 and 40 cycles RT-PCR
++ detectable at less than 35 cycles RT-PCR with SE greater than 0.9
+++ detectable at less than 35 cycles RT-PCR with SE less than 0.9
All RT-PCR was performed on the Applied Biosystems 7500 Real-Time PCR System

TABLE VII-C
THE EFFECT OF TWO 1.25 μM TRICHOSTATIN A DOSES ON
GENE EXPRESSION IN AN EARLY PASSAGE (P5)
PANCREATIC-DERIVED STROMAL CELL LINE.

| sample | glucagon | HNF | Insulin | Pdx-1 |
|---|---|---|---|---|
| untreated | nd | nd | nd | + |
| 24 hrs | +++ | +++ | nd | +++ |
| 48 hrs | + | nd | nd | nd |
| 72 hrs | nd | nd | nd | nd |
| 96 hrs | nd | nd | nd | nd |
| 120 hrs | + | +++ | nd | +++ |
| 144 hrs | + | nd | nd | nd |

nd not detectable at >40 cycles by RT-PCR
+ detectable between 35 and 40 cycles by RT-PCR
++ detectable at less than 35 cycles by RT-PCR with SE greater than 0.9
+++ detectable at less than 35 cycles by RT-PCR with SE less than 0.9
All RT-PCR was performed on the Applied Biosystems 7500 Real-Time PCR System

TABLE VII-D
THE EFFECT OF TWO 1.25 μM TRICHOSTATIN A DOSES ON
GENE EXPRESSION IN A LATE PASSAGE (P14)
PANCREATIC-DERIVED STROMAL CELL LINE.

| sample | glucagon | HNF | Insulin | Pdx-1 |
|---|---|---|---|---|
| untreated | nd | nd | nd | nd |
| 24 hrs | +++ | + | nd | +++ |
| 48 hrs | +++ | nd | +− | nd |
| 72 hrs | + | nd | nd | nd |
| 96 hrs | nd | nd | nd | nd |
| 120 hrs | = | +++ | nd | ++ |
| 144 hrs | + | nd | nd | nd |

nd not detectable at >40 cycles by RT-PCR
+ detectable between 35 and 40 cycles by RT-PCR
++ detectable at less than 35 cycles by RT-PCR with SE greater than 0.9
+++ detectable at less than 35 cycles by RT-PCR with SE less than 0.9
All RT-PCR was performed on the Applied Biosystems 7500 Real-Time PCR System

TABLE VIII-A
THE EFFECT OF TWO 1.25 μM TRICHOSTATIN A DOSES
ON GENE EXPRESSION IN AN AMNIOTIC
FLUID-DERIVED CELL LINE.

| sample | Gata1 | HNF-3 beta | insulin | Pdx-1 | sox17 |
|---|---|---|---|---|---|
| untreated | nd | nd | + | + | +++ |
| 24 hrs | + | +++ | nd | +++ | +++ |
| 48 hrs | nd | +++ | nd | nd | +++ |
| 72 hrs | nd | + | + | nd | +++ |
| 96 hrs | nd | + | + | nd | +++ |
| 120 hrs | nd | +++ | nd | +++ | +++ |
| 144 hrs | nd | +++ | nd | nd | +++ |

nd not detectable at >40 cycles by RT-PCR
+ detectable between 35 and 40 cycles by RT-PCR
++ detectable at less than 35 cycles by RT-PCR with SE greater than 0.9
+++ detectable at less than 35 cycles by RT-PCR with SE less than 0.9
All RT-PCR was performed on the Applied Biosystems 7500 Real-Time PCR System

TABLE VIII-B
THE EFFECT OF TWO 1.25 μM TRICHOSTATIN A DOSES
ON GENE EXPRESSION IN A SECOND AMNIOTIC
FLUID-DERIVED CELL LINE.

| sample | Gata1 | HNF-3 beta | insulin | Pdx-1 | sox17 |
|---|---|---|---|---|---|
| untreated | nd | nd | +++ | nd | +++ |
| 24 hrs | + | +++ | nd | +++ | +++ |
| 48 hrs | nd | +++ | nd | nd | +++ |
| 72 hrs | nd | +++ | + | nd | +++ |
| 96 hrs | nd | + | + | nd | +++ |
| 120 hrs | + | +++ | nd | +++ | +++ |
| 144 hrs | nd | +++ | nd | nd | +++ |

nd not detectable at >40 cycles by RT-PCR
+ detectable between 35 and 40 cycles by RT-PCR
++ detectable at less than 35 cycles by RT-PCR with SE greater than 0.9
+++ detectable at less than 35 cycles by RT-PCR with SE less than 0.9
All RT-PCR was performed on the Applied Biosystems 7500 Real-Time PCR System

TABLE VIII-C
THE EFFECT OF TWO 5 μM TRICHOSTATIN A DOSES ON
GENE EXPRESSION IN AN EARLY PASSAGE (P5)
PANCREATIC-DERIVED STROMAL CELL LINE.

| sample | glucagon | HNF-3 beta | Insulin | Pdx-1 |
|---|---|---|---|---|
| untreated | nd | nd | nd | + |
| 24 hrs | +++ | +++ | nd | ++ |
| 48 hrs | + | nd | nd | nd |
| 72 hrs | + | nd | nd | nd |
| 96 hrs | nd | nd | nd | nd |
| 120 hrs | nd | + | nd | +++ |
| 144 hrs | + | nd | nd | nd |

nd not detectable at >40 cycles by RT-PCR
+ detectable between 35 and 40 cycles by RT-PCR
++ detectable at less than 35 cycles by RT-PCR with SE greater than 0.9
+++ detectable at less than 35 cycles by RT-PCR with SE less than 0.9
All RT-PCR was performed on the Applied Biosystems 7500 Real-Time PCR System

TABLE VIII-D
THE EFFECT OF TWO 5 μM TRICHOSTATIN A DOSES ON
GENE EXPRESSION IN A LATE PASSAGE (P11)
PANCREATIC-DERIVED STROMAL CELL LINE.

| sample | glucagon | HNF-3 beta | Insulin | Pdx-1 |
|---|---|---|---|---|
| untreated | nd | nd | nd | nd |
| 24 hrs | +++ | +++ | nd | + |
| 48 hrs | + | nd | + | nd |
| 72 hrs | + | nd | nd | nd |
| 96 hrs | + | nd | nd | nd |
| 120 hrs | + | + | nd | +++ |
| 144 hrs | + | nd | nd | nd |

nd not detectable
− detectable at greater than 35 cycles
+ detectable at less than 35 cycles with SE greater than 0.9
++ detectable at less than 35 cycles with SE less than 0.9
All RT-PCR was performed on the Applied Biosystems 7500 Real-Time PCR System

TABLE IX-A
THE EFFECT OF TWO 1.25 μM TRICHOSTATIN A DOSES
ON GENE EXPRESSION IN AN AMNIOTIC
FLUID-DERIVED CELL LINE.

| sample | Gata1 | HNF-3 beta | insulin | Pdx-1 | sox17 |
|---|---|---|---|---|---|
| untreated | nd | nd | + | + | +++ |
| 24 hrs | + | +++ | nd | +++ | +++ |
| 48 hrs | nd | +++ | nd | +++ | +++ |
| 72 hrs | nd | + | nd | nd | +++ |
| 96 hrs | nd | nd | nd | nd | +++ |
| 120 hrs | nd | + | nd | + | +++ |

nd not detectable at >40 cycles by RT-PCR
+ detectable between 35 and 40 cycles by RT-PCR
++ detectable at less than 35 cycles by RT-PCR with SE greater than 0.9
+++ detectable at less than 35 cycles by RT-PCR with SE less than 0.9
All RT-PCR was performed on the Applied Biosystems 7500 Real-Time PCR System

TABLE IX-B
THE EFFECT OF TWO 1.25 μM TRICHOSTATIN A DOSES
ON GENE EXPRESSION IN A SECOND AMNIOTIC
FLUID-DERIVED CELL LINE.

| sample | Gata1 | HNF-3 beta | insulin | Pdx-1 | sox17 |
|---|---|---|---|---|---|
| untreated | nd | nd | +++ | nd | +++ |
| 24 hrs | + | +++ | nd | ++ | +++ |
| 48 hrs | nd | +++ | nd | + | +++ |
| 72 hrs | nd | +++ | nd | nd | +++ |
| 96 hrs | nd | + | nd | nd | +++ |
| 120 hrs | + | +++ | nd | +++ | +++ |

nd not detectable at >40 cycles by RT-PCR
+ detectable between 35 and 40 cycles by RT-PCR
++ detectable at less than 35 cycles by RT-PCR with SE greater than 0.9
+++ detectable at less than 35 cycles by RT-PCR with SE less than 0.9
All RT-PCR was performed on the Applied Biosystems 7500 Real-Time PCR System

TABLE IX-C
THE EFFECT OF TWO 5 μM TRICHOSTATIN A DOSES ON
GENE EXPRESSION IN AN EARLY PASSAGE (P5)
PACNREATIC-DERIVED STROMAL CELL LINE.

| sample | glucagon | HNF-3 beta | Insulin | Pdx-1 |
|---|---|---|---|---|
| untreated | nd | nd | nd | + |
| 24 hrs | +++ | +++ | nd | +++ |
| 48 hrs | +++ | +++ | nd | +++ |
| 72 hrs | + | nd | +++ | nd |
| 96 hrs | + | nd | nd | nd |
| 120 hrs | + | +++ | nd | +++ |

nd not detectable at >40 cycles by RT-PCR
+ detectable between 35 and 40 cycles by RT-PCR
++ detectable at less than 35 cycles by RT-PCR with SE greater than 0.9
+++ detectable at less than 35 cycles by RT-PCR with SE less than 0.9
All RT-PCR was performed on the Applied Biosystems 7500 Real-Time PCR System

TABLE IX-D
THE EFFECT OF TWO 5 μM TRICHOSTATIN A DOSES ON
GENE EXPRESSION IN A LATE PASSAGE (P11)
PACNREATIC-DERIVED STROMAL CELL LINE.

| sample | glucagon | HNF-3 beta | Insulin | Pdx-1 |
|---|---|---|---|---|
| untreated | nd | nd | nd | nd |
| 24 hrs | +++ | +++ | nd | + |
| 48 hrs | +++ | +++ | nd | +++ |
| 72 hrs | + | nd | nd | nd |
| 96 hrs | nd | nd | nd | nd |
| 120 hrs | + | +++ | + | +++ |

nd not detectable at >40 cycles by RT-PCR
+ detectable between 35 and 40 cycles by RT-PCR
++ detectable at less than 35 cycles by RT-PCR with SE greater than 0.9
+++ detectable at less than 35 cycles by RT-PCR with SE less than 0.9
All RT-PCR was performed on the Applied Biosystems 7500 Real-Time PCR System

TABLE X-A
THE EFFECT OF A 6 HOUR 1.25 μM TRICHOSTATIN A
TREATMENT ON GENE EXPRESSION IN AN AMNIOTIC
FLUID-DERIVED CELL LINE.

| sample | Gata1 | HNF-3 beta | insulin | Pdx-1 | sox17 |
|---|---|---|---|---|---|
| untreated | nd | nd | + | + | +++ |
| 6 hrs | nd | +++ | nd | + | +++ |
| 24 hrs | nd | +++ | nd | nd | +++ |

nd not detectable at >40 cycles by RT-PCR
+ detectable between 35 and 40 cycles by RT-PCR
++ detectable at less than 35 cycles by RT-PCR with SE greater than 0.9
+++ detectable at less than 35 cycles by RT-PCR with SE less than 0.9
All RT-PCR was performed on the Applied Biosystems 7500 Real-Time PCR System

TABLE X-B
THE EFFECT OF A 6 HOUR 1.25 μM TRICHOSTATIN A
TREATMENT ON GENE EXPRESSION IN A SECOND
AMNIOTIC FLUID-DERIVED CELL LINE.

| sample | Gata1 | HNF-3 beta | insulin | Pdx-1 | sox17 |
|---|---|---|---|---|---|
| untreated | nd | nd | +++ | nd | +++ |
| 6 hrs | nd | +++ | nd | +++ | +++ |
| 24 hrs | nd | + | nd | nd | +++ |

nd not detectable at >40 cycles by RT-PCR
+ detectable between 35 and 40 cycles by RT-PCR
++ detectable at less than 35 cycles by RT-PCR with SE greater than 0.9
+++ detectable at less than 35 cycles by RT-PCR with SE less than 0.9
All RT-PCR was performed on the Applied Biosystems 7500 Real-Time PCR System

TABLE X-C
THE EFFECT OF A 6 HOUR 5 μM TRICHOSTATIN A
TREATMENT ON GENE EXPRESSION IN AN EARLY PASSAGE
(P5) PANCREATIC-DERIVED STOMAL CELL LINE.

| sample | glucagon | HNF-3 beta | Insulin | Pdx-1 |
|---|---|---|---|---|
| untreated | nd | nd | nd | + |
| 6 hrs | + | +++ | nd | + |
| 24 hrs | + | nd | +++ | nd |

nd not detectable at >40 cycles by RT-PCR
+ detectable between 35 and 40 cycles by RT-PCR
++ detectable at less than 35 cycles by RT-PCR with SE greater than 0.9
+++ detectable at less than 35 cycles by RT-PCR with SE less than 0.9
All RT-PCR was performed on the Applied Biosystems 7500 Real-Time PCR System

TABLE X-D
THE EFFECT OF A 6 HOUR 5 μM TRICHOSTATIN A
TREATMENT ON GENE EXPRESSION IN A LATE PASSAGE
(P11) PANCREATIC-DERIVED STOMAL CELL LINE.

| sample | glucagon | HNF-3 beta | Insulin | Pdx-1 |
|---|---|---|---|---|
| untreated | nd | nd | nd | nd |
| 6 hrs | + | + | nd | + |
| 24 hrs | + | nd | nd | nd |

nd not detectable at >40 cycles by RT-PCR
+ detectable between 35 and 40 cycles by RT-PCR
++ detectable at less than 35 cycles by RT-PCR with SE greater than 0.9
+++ detectable at less than 35 cycles by RT-PCR with SE less than 0.9
All RT-PCR was performed on the Applied Biosystems 7500 Real-Time PCR System

TABLE XI-A
ANTIBODIES TO SURFACE RECEPTORS

| Antibody | Supplier | Isotype | Clone |
|---|---|---|---|
| Alkaline | R&D systems | Mouse IgG1 | B4-78 |
| phosphatase | (MN) | | |
| ATP binding | BD Pharmingen | Mouse IgG2b, | 5D3 |
| cassette transporter | (CA) | Kappa | |
| (ABCG2) | | | |
| CD10 | BD Pharmingen | Mouse IgG1, | HI10a |
| | (CA) | Kappa | |
| CD29 (Beta 1 | BD Pharmingen | Mouse IgG1, | MAR4 |
| integrin) | (CA) | Kappa | |
| CD44 | BD Pharmingen | Mouse IgG2b, | G44-26 |
| | (CA) | Kappa | |
| CD45 | BD Pharmingen | Mouse IgG1, | Hi30 |
| | (CA) | Kappa | |
| CD49f | BD Pharmingen | Rat IgG2A, Kappa | G0H3 |
| | (CA) | | |
| CD49b (Alpha 2 | BD Pharmingen | Mouse IgG2a, | 121-H6 |
| integrin) | (CA) | Kappa | |
| CD56 (NCAM) | BD Pharmingen | Mouse IgG1, | B159 |
| | (CA) | Kappa | |
| CD73 | BD Pharmingen | Mouse IgG1, | AD2 |
| | (CA) | Kappa | |
| CD90 | BD Pharmingen | Mouse IgG1, kappa | 5E10 |
| | (CA) | | |
| CD95 | BD Pharmingen | Mouse IgG1, | DX2 |
| | (CA) | Kappa | |
| CD105 (endoglin) | Santa Cruz | Mouse IgG1 | P3D1 |
| | Biotechnology | | |
| | (CA) | | |
| CD117 (c-Kit) | BD Pharmingen | Mouse IgG1, kappa | YB5.B8 |
| | (CA) | | |
| CD133 | Miltenyi Biotec | Mouse IgG1 | Ac133 |
| | (CA) | | |
| Epithelial adhesion | BD Pharmingen | Mouse IgG1 | EBA-1 |
| molecule (EpCAM) | (CA) | | |
| Hepatocyte growth | R&D systems | Mouse IgG2A | 95309 |
| factor receptor | (MN) | | |
| (HGF or c-Met) | | | |
| Platelet/endothelial | Santa Cruz | Mouse IgG1 | WM-59 |
| cell adhesion | Biotechnology | | |
| molecule-1 | (CA) | | |
| (PECAM-1) | | | |
| CD49b (Alpha 2 | BD Pharmingen | Mouse IgG2a, | 121-H6 |
| integrin) | (CA) | Kappa | |
| Alpha 3 integrin | Santa Cruz (CA) | Mouse IgG1 | P1B5 |
| Alpha 5 intgerin | Santa Cruz (CA) | Mouse IgG3 | P1D6 |
| Beta 3 integrin | Santa Cruz (CA) | Mouse IgG1 | Y2/51 |
| Alpha V Beta 3 | BD Pharmingen | Mouse IgG1, | 23C6 |
| integrin (CD51/61) | (CA) | Kappa | |
| SSEA-3 | Chemicon (CA) | Mouse IgG3 | MC-631 |
| SSEA-4 | Chemicon (CA) | Rat IgM | MC-813- |
| | | | 70 |
| TRA 1-60 | Chemicon (CA) | Mouse IgM | TRA 1-60 |
| TRA 1-81 | Chemicon (CA) | Mouse IgM | TRA 1-81 |
| TRA 1-85 | Chemicon (CA) | Mouse IgG1 | TRA 1-85 |
| TRA 2-54 | Chemicon (CA) | Mouse IgG1 | TRA 2-54 |
| EGF r | BD Pharmingen | Mouse IgG2b, | EGFR1 |
| | (CA) | Kappa | |
| HLA ABC | BD Pharmingen | Mouse IgG1, | G46-2.6 |
| | (CA) | Kappa | |
| HLA DR | BD Pharmingen | Mouse IgG2b, | TU36 |
| | (CA) | Kappa | |

TABLE XI-B
LIST OF ANTIBODIES USED FOR IDENTIFICATION OF
INTRACELLULAR MARKERS

| Antibody | Supplier | Isotype | Clone |
|---|---|---|---|
| Nestin | R&D systems | Mouse IgG1 | HSG02 |
| | (MN) | | |
| Cytokeratin 5/8 | Santa Cruz | Mouse IgG1 | C50 |
| | Biotechnology | | |
| | (CA) | | |
| Vimentin | Santa Cruz | Mouse IgG1 | V9 |
| | Biotechnology | | |
| | (CA) | | |
| Pan-Cytokeratin (4, | Santa Cruz | Mouse IgG1 | C11 |
| 5, 6, 8, 10, 13, 18) | Biotechnology | | |
| | (CA) | | |
| Peripherin | Santa Cruz | Goat Polyclonal | C19 |
| | Biotechnology | | |
| | (CA) | | |
| Gilial fibrillary | Santa Cruz | Goat polyclonal | N-18 |
| acidic protein | Biotechnology | | |
| (GFAP) | (CA) | | |
| Pan-Cytokeratin | BD Pharmingen | Mouse IgG1, | KA4 |
| (14, 15, 16, and 19) | (CA) | Kappa | |
| Beta III tubulin | Chemicon | Mouse IgG1 | TU-20 |
| | International (CA) | | |

TABLE XII
LIST OF SECONDARY CONJUGATED ANTIBODIES USED FOR
FACS AND IMMUNOSTAINININGANALYSIS.

| Secondary conjugated | | |
|---|---|---|
| antibody | Supplier | Dilution |
| Goat Anti-Mouse IgG APC | Jackson ImmunoResearch | 1:200 |
| conjugated | (PA) | |
| Goat Anti-Mouse IgG PE | Jackson ImmunoResearch | 1:200 |
| conjugated | (PA) | |
| Donkey anti-rabbit PE or - | Jackson ImmunoResearch | 1:200 |
| APC conjugated | (PA) | |
| Donkey anti-goat PE or - | Jackson ImmunoResearch | 1:200 |
| APC conjugated | (PA) | |
| Goat anti-mouse IgM PE | SouthernBiotech (AL) | 1:200 |
| Goat anti-Rat IgM PE | SouthernBiotech (AL) | 1:200 |
| Goat anti-mouse IgG3 PE | SouthernBiotech (AL) | 1:200 |

TABLE XIII
THE EFFECTS OF HISTONE DEACETLYASE INHIBITOR
TREATMENT ON GENE EXPRESSION IN RESTING PERIPHERAL
BLOOD MONONUCLEAR CELLS (PBMCS) AND PBMCS TREATED
WITH THE MITOGENIC LECTIN PHA.

| | GAPDH | GATA1 | GATA4 | Pdx-1 |
|---|---|---|---|---|
| 24 hrs | | | | |
| untreated resting PBMCs | +++ | +++ | nd | nd |
| resting PBMC 5 μM TSA | +++ | nd | + | nd |
| PHA treated PBMCs | +++ | + | ++ | nd |
| without TSA | | | | |
| PHA treated PBMCs with | +++ | nd | +++ | + |
| 5 μM TSA | | | | |
| 48 hours | | | | |
| untreated resting PBMCs | +++ | + | nd | nd |
| resting PBMC 5 μM TSA | +++ | nd | + | nd |
| PHA treated PBMCs | +++ | + | nd | nd |
| without TSA | | | | |
| PHA treated PBMCs with | ++++ | nd | +++ | + |
| 5 μM TSA | | | | |

nd not detectable at >40 cycles by RT-PCR
+ detectable at greater than 35–40 cycles by RT-PCR
++ detectable at less than 35 cycles by RT-PCR with SE greater than 0.9
+++ detectable at less than 35 cycles by RT-PCR with SE less than 0.9
All RT-PCR was performed on the Applied Biosystems 7500 Real-Time PCR System
GATA 1 is a hematopoietic lineage marker
GATA4 is a mesenchymal and endodermal lineage marker

Publications cited throughout this document are hereby incorporated by reference in their entirety. Although the various aspects of the invention have been illustrated above by reference to examples and preferred embodiments, it will be appreciated that the scope of the invention is defined not by the foregoing description, but by the following claims properly construed under principles of patent law.

What is claimed is:

1. A method for promoting the differentiation of cells, comprising the steps of:
a. Providing cells, and
b. Contacting the cells with at least one chromatin-remodeling agent, wherein the at least one chromatin-remodeling agent increases the expression of a transcriptional regulator.

2. The method of claim 1 wherein the transcriptional regulator is PDX-1.

3. The method of claim 1, wherein the cells do not express the transcriptional regulator prior to the treatment with the at least one chromatin-remodeling agent.

4. The method of claim 1, wherein the treatment of at least one chromatin-remodeling agent restores the expression of the transcriptional regulator.

5. The method of claim 1, wherein the treatment of the cells with the at least one chromatin-remodeling agent causes increases in the expression of at least one of the genes HNF-3 beta or Sox-17.

6. The method of claim 1, wherein the cells are selected from the group consisting of an undifferentiated cell, a partially differentiated cell, and a fully differentiated cell.

7. The method of claim 1, wherein the at least one chromatin-remodeling agent is an inhibitor of histone deacetylase activity.

8. The method of claim 7, wherein the inhibitor is selected from the group consisting of butyrates, hydroxamic acids, cyclic peptides and benzamides.

9. The method of claim 7, wherein the inhibitor is selected from the group consisting of valproic acid, 4-phenylbutyrate, sodium butyrate, trichostatin A, suberoyl anilide hydroxamic acid (SAHA), oxamflatin, trapoxin B, FR901228, apicidin, chlamydocin, depuecin, scriptaid, depsipeptide, and N-acetyldinaline.

10. A method for promoting the differentiation of cells into a pancreatic hormone-secreting cell, comprising the steps of:
a. Providing cells, and
b. Contacting the cells with at least one chromatin-remodeling agent, wherein the chromatin-remodeling agent increases the expression of a transcriptional regulator.

11. The method of claim 10 wherein the transcriptional regulator is PDX-1.

12. The method of claim 10, wherein the cells do not express the transcriptional regulator prior to the treatment with the at least one chromatin-remodeling agent.

13. The method of claim 10, wherein the treatment of at least one chromatin-remodeling agent restores the expression of the transcriptional regulator.

14. The method of claim 10, wherein the treatment of the cells with the at least one chromatin-remodeling agent causes increases in the expression of at least one of the genes HNF-3 beta, Sox-17, insulin, glucagon, or somatostatin.

15. The method of claim 10, wherein the cells are selected from the group consisting of an undifferentiated cell, a partially differentiated cell, and a fully differentiated cell.

16. The method of claim 10, wherein the at least one chromatin-remodeling agent is an inhibitor of histone deacetylase activity.

17. The method of claim 16, wherein the inhibitor is selected from the group consisting of butyrates, hydroxamic acids, cyclic peptides and benzamides.

18. The method of claim 16, wherein the inhibitor is selected from the group consisting of valproic acid, 4-phenylbutyrate, sodium butyrate, trichostatin A, suberoyl anilide hydroxamic acid (SAHA), oxamflatin, trapoxin B, FR901228, apicidin, chlamydocin, depuecin, scriptaid, depsipeptide, and N-acetyldinaline.

19. A method for increasing the expression of PDX-1 in cells, comprising the steps of:
a. Providing the cells, and
b. Contacting the cells with at least one chromatin-remodeling agent, wherein the at least one chromatin-remodeling agent increases the expression of a transcriptional regulator within the cells.

20. The method of claim 19, wherein the cells do not express PDX-1 prior to the treatment with the at least one chromatin-remodeling agent.

21. The method of claim 19, wherein the treatment of at least one chromatin-remodeling agent restores the expression of PDX-1.

22. The method of claim 19, wherein the cells are selected from the group consisting of an undifferentiated cell, a partially differentiated cell, and a fully differentiated cell.

23. The method of claim 19, wherein the at least one chromatin-remodeling agent is an inhibitor of histone deacetylase activity.

24. The method of claim 23, wherein the inhibitor is selected from the group consisting of butyrates, hydroxamic acids, cyclic peptides and benzamides.

25. The method of claim 23, wherein the inhibitor is selected from the group consisting of valproic acid, 4-phenylbutyrate, sodium butyrate, trichostatin A, suberoyl anilide hydroxamic acid (SAHA), oxamflatin, trapoxin B, FR901228, apicidin, chlamydocin, depuecin, scriptaid, depsipeptide, and N-acetyldinaline.

26. A method of treating a treating a disease, comprising the steps of:
a. Providing cells that do not express a specific transcriptional regulator,
b. Contacting the cells with at least one chromatin-remodeling agent to increase the expression of the specific transcriptional-remodeling agent,
c. Allowing the cells to differentiate into a cell of pancreatic lineage, and
d. Transplanting such cells into a patient.

27. The method of claim 26, wherein the transcriptional regulator is PDX-1.

28. The method of claim 26, wherein the cells express the transcriptional regulator in insufficient amounts to cause the cell to differentiate when differentiation protocols are applied.

29. The method of claim 26, wherein contacting the cells with the at least one chromatin-remodeling agent restores the expression of the specific transcriptional regulator.

30. The method of claim 26, wherein the treatment of the cells with the at least one chromatin-remodeling agent causes increases in the expression of at least one of the genes HNF-3 beta or Sox-17.

31. The method of claim 26, wherein the at least one chromatin-remodeling agent is an inhibitor of histone deacetylase activity.

32. The method of claim 31, wherein the inhibitor is selected from the group consisting of butyrates, hydroxamic acids, cyclic peptides and benzamides.

33. The method of claim 31, wherein the inhibitor is selected from the group consisting of valproic acid, 4-phenylbutyrate, sodium butyrate, trichostatin A, suberoyl anilide hydroxamic acid (SAHA), oxamflatin, trapoxin B, FR901228, apicidin, chlamydocin, depuecin, scriptaid, depsipeptide, and N-acetyldinaline.
